(19) United States
(12) Patent Application Publication
(10) Pub. No.: US 2018/0179499 A1
(43) Pub. Date: Jun. 28, 2018

(54) BIOBASED PRODUCTION OF FUNCTIONALIZED ALPHA-SUBSTITUTED ACRYLATES AND C4-DICARBOXYLATES

(71) Applicant: BIOAMBER INC., St. Paul, MN (US)

(72) Inventors: Man Kit LAU, Minneapolis, MN (US); Cathy Staloch HASS, Saint Louis Park, MN (US); Hayat Nuru AHMED, Minneapolis, MN (US); Ralph Thomas BAKER, Ottawa (CA); James R. MILLIS, Plymouth, MN (US); Indira THAPA, Ottawa (CA)

(21) Appl. No.: 15/578,754

(22) PCT Filed: Jun. 3, 2016
(86) PCT No.: PCT/US2016/035765

§ 371 Date: Dec. 1, 2017

Related U.S. Application Data
(60) Provisional application No. 62/171,019, filed on Jun. 4, 2015.
(60) Provisional application No. 62/171,029, filed on Jun. 4, 2015.

(51) Int. Cl. C12N 9/10; C12P 7/46; C12P 7/58; C12N 9/16; C12P 7/42; C12N 9/88; C07C 51/38; C07C 51/377; C07C 59/185; C07C 59/147; C12P 7/62

(52) U.S. Cl. CPC C12N 9/1096; C12P 7/62; C12N 9/1025; C12Y 203/03014; C12P 7/46; C12P 7/58; C12Y 301/0306; C12N 9/16; C12P 7/42; C12N 9/88; C12Y 402/01003; C07C 51/38; C07C 51/377; C07C 59/185; C07C 59/147; C12Y 206/01045

(57) ABSTRACT

The description provides, inter alia, recombinant microorganisms, engineered metabolic pathways, chemical catalysts, and products produced through the use of the described methods and materials. The products produced include functionalized alpha substituted C4 dicarboxylic acids and functionalized acrylic acids and salts, esters and lactones thereof.

### CROSS-REFERENCE TO RELATED APPLICATIONS

This application claims the benefit of U.S. Provisional Patent Application No. 62/171,019, filed Jun. 4, 2015, and U.S. Provisional Patent Application No. 62/171,029, filed Jun. 4, 2015, both of which are incorporated herein by reference.

### FIELD

The description provides, inter alia, recombinant microorganisms, engineered metabolic pathways, chemical catalysts, and products produced through the use of the described methods and materials. The products produced include functionalized alpha substituted C4 dicarboxylic acids and functionalized acrylic acids and salts, esters and lactones thereof.

### BACKGROUND

Currently, many carbon-containing chemicals are derived from petroleum based sources. Reliance on petroleum-derived feedstocks contributes to depletion of petroleum reserves and the harmful environmental impact associated with oil drilling.

Certain carbonaceous products of sugar fermentation are seen as replacements for petroleum-derived materials for use as feedstocks for the manufacture of carbon-containing chemicals. Such products include functionalized alpha substituted acrylic acids, for example including hydroxymethyl acrylic acid, hydroxyethyl acrylic acid, isopropyl acrylic acid, and others. Functionalized acrylic acids represent a growing market for which all commercial production today is petroleum-derived.

### SUMMARY

The disclosure provides recombinant microorganisms that have been designed to produce functionalized alpha substituted C4 dicarboxylic acids and functionalized alpha substituted 3-hydroxypropionic acid. Throughout the description and the claims the mentions of an organic acid shall include salts, esters and lactones thereof unless the context clearly indicates otherwise. The intermediates in these pathways as well as the final products from these pathways are useful, among other things, for use in the chemical industry for example as a component of a polymer. The products and intermediates from these pathways are also useful in either their protected or unprotected form as substrates for further chemical reactions. For example, derivatives of functionalized alpha substituted C4 carboxylic acids are described that can be made through chemical reaction. In some examples, functionalized alpha substituted acrylic acids are produced through the use of metal catalysts and/or dehydration reactions.

In some instances the recombinant microorganisms are engineered to include a pathway that produces a functionalized alpha substituted dicarboxylic acid and/or a functionalized alpha substituted 3HP. One of ordinary skill in the art will appreciate that upon expression of a given pathway the end product of the pathway as well as some of the intermediates within the pathway will be created and isolated from the fermentation. In some instances the mixture of such products can be used in a subsequent reaction to create an end product. Some of the engineered pathways described herein include the expression of one or more polypeptides having decarboxylase activity (See FIG. 11 and FIG. 12, row I) or a 3HP CoA dehydratase (See FIG. 13 and FIG. 14, row G).

Other embodiments include recombinant microorganisms that are engineered to produce compounds selected from 2-methylene-succinyl semialdehyde, alpha-hydroxyethyl acrylate and tulipalin A. Such recombinant microorgansims include nucleic acid sequences encoding one or more enzymes having the following activities: oxi-reductase, reductase, cyclase, lactonase, and lactamase activity. Also taught herein are specific amino acid sequences that can be used to arrive at these activities and recombinant microorgansims for example oxi-reductase (FIG. 18, row A), reductase (FIG. 18, row B), cyclase (FIG. 18, row C), lactonase (FIG. 18, row C), and lactamase (FIG. 18, row C). These recombinant microorganism can also be selected or engineered such that they remain viable in the presence of at least 0.5, 0.75, 1.0, 3.0, 5.0, 7.0, or 10 g/L of a compound selected from 2-methylene-succinyl semialdehyde, alpha-hydroxyethyl acrylate and tulipalin A. These recombinant microorganisms can produce one or more of the following products at a rate of at least 0.02, 0.1, 0.2, 0.5, 1.0, 1.5, 3.0, 5.0, 7.0, or 10.0 g/L/hr of itaconic acid, 2-methylene-succinyl semialdehyde, alpha-hydroxyethyl acrylate or tulipalin A. Methods of making these products including fermenting the recombinant microorganism in a fermentation broth and separating the product(s) from the fermentation broth.

One of ordinary skill in the art will appreciate that impurities will co-purify with desired products and that one or more processing steps such as ion exchange, distillation, crystallization, and the like may be used to reduce the impurity levels. The actual level of purity desired will be a balance between the cost of purifying and the tolerance of the end use for the impurity. For example, less than 0.01 ppm of an impurity may be needed for some applications, however, impurities ranging from less than 0.01, 50, 100, 150, 200, or greater than 300 ppm may be acceptable. Impurities may be in the form of carbohydrates, salts, metals, gases, organic matter, cellular debris, and combinations thereof. In some instance impurities may have desirable side effects.

Recombinant microorganisms described herein have been engineered to express or overexpress a polypeptide that has certain enzymatic activity. The enzymatic activity is generally described as the ability to convert a substrate to a product. One of ordinary skill in the art will appreciate that enzymes can be structurally unrelated, use divergent cofactors and have different ancestry but yet are capable of converting the same substrates into the same products. In light of this, the disclosure comprises a recombinant microorganism that includes through recombinant techniques the specified enzymatic activity (see for example the tables of FIG. 4 and FIG. 6) regardless of its structural similarity to another polypeptide having the same activity. These recombinant microorganisms will also include the product produced at a higher concentration than that found in the native host microorganism. In some instances, the product will not be found in the native host microorganism.

Exemplary recombinant microorganisms include products such as functionalized alpha substituted C4 dicarboxylic acids, wherein the functional alpha substitution is selected from an alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, n>3, or n>4, and at least one recombinant nucleic acid sequence encoding at least one enzyme selected from a transaminase (FIG. 4, row A), a synthase (FIG. 4, row B), a dehydratase (FIG. 4, row C), a hydratase (FIG. 4, row D), a dehydrogenase (FIG. 4, row E), a cis-trans isomerase (FIG. 4, row F), a cyclase, lactonase, lactamase (FIG. 4, row G), an esterase (FIG. 4, row H), a reductase (FIG. 6, row C), a decarboxylase (FIG. 6, row D) and an aldehyde reductase (FIG. 6, row E). One of ordinary skill in the art will appreciate that the cyclase, lactonase, lactamase and/or esterase can be used to enzymatically convert the alpha substituted acrylic acid product to its lactone or ester form following the catalysis reaction. The functionalized alpha substituted C4 dicarboxylic acid can be an alpha-(hydroxymethyl) malic acid, an alpha-(2-hydroxypropyl) malic acid, an alpha-(1-hydroxyethyl) malic acid and/or an alpha-(2-hydroxyethyl) malic acid. Depending upon the engineered pathway the recombinant microorganism can include 1, 2, 3, 4, or more functionalized alpha substituted C4 dicarboxylic acids (See the pathways in FIG. 3 and FIG. 9). One of ordinary skill in the art will appreciate that in instances where the spacer pathway is used to elongate the alpha substitution group there can be several distinct functionalized alpha substituted C4 dicarboxylic acids. In many instances a given recombinant microorganism will include multiple functionalized alpha substituted C4 dicarboxylic acids, such as malic acid, fumaric acid and the like, and those multiple functionalized alpha substituted C4 dicarboxylic acids will contain the same functional alpha substitution. (See FIGS. 3, 5, 9, and 10).

Exemplary recombinant microorganisms include products such as functionalized alpha substituted acrylic acids, wherein the functional alpha substitution is selected from an alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, n>3, or n>4, and at least one recombinant nucleic acid sequence encoding at least one enzyme selected from a transaminase (FIG. 12, row A), a synthase (FIG. 12, row B), a dehydratase (FIG. 12, row C), a hydratase (FIG. 12, row D), a dehydrogenase (FIG. 12, row E), a cis-trans isomerase (FIG. 12, row F), a cyclase, lactonase, lactamase (FIG. 12, row G), an esterase (FIG. 12, row H), a decarboxylase (FIG. 12, row I), a reductase (FIG. 14, row C), a decarboxylase (FIG. 14, row D), a reductase (FIG. 14, row E), a CoA transferase (FIG. 14, row F), and a 3HP-CoA dehydratase (FIG. 14, row G). The functionalized alpha substituted acrylic acid can be an alpha-(hydroxymethyl)acrylic acid, an alpha-(2-hydroxypropyl)acrylic acid, an alpha-(1-hydroxyethyl)acrylic acid and/or an alpha-(2-hydroxyethyl)acrylic acid. One of ordinary skill in the art will appreciate that in instances where the spacer pathway is used to elongate the alpha substitution group there can be several distinct functionalized alpha substituted acrylic acids having varying carbon chain lengths in the functional group.

In some embodiments the functional group that is present in the functionalized alpha substituted C4 dicarboxylic acids can originate from an amino acid. FIG. 7 shows these functional groups and one of ordinary skill in the art will appreciate that in the event that a functionalized alpha substituted C4 dicarboxylic acid is produced from a given amino acid and the functionalized alpha substituted C4 dicarboxylic acids is subsequently catalytically converted to an acrylate that the methylene group will occupy the location of the original amine in the amino acid. In instances where an amino acid is selected as a substrate for the engineered pathway it may be convenient to choose a microorganism that has already been engineered to overproduce that amino acid or naturally overproduces that amino acid.

In some embodiments the functional group that is present in the functionalized alpha substituted acrylic acid can originate from an amino acid. FIG. 7 shows these functional groups and one of ordinary skill in the art will appreciate that the methylene group in the functionalized alpha substituted acrylic acid will occupy the same position as the amino group from the amino acid. In instances where an amino acid is selected as a substrate for the engineered pathway it may be convenient to choose a microorganism that has already been engineered to overproduce that amino acid or naturally overproduces that amino acid.

In some embodiments the recombinant microorganism will use a naturally occurring dicarboxylic acid as a substrate for subsequent conversion to a functionalized alpha substituted C4 acrylic acid. For example, the host organism can produce naturally a 3-phosphohydroxypyruvate (see FIG. 9) or itaconic acid (see FIG. 9) or itatartaric acid (see FIG. 9) and include increased phosphatase (E.C. 3.1.3.21) or itaconic oxidase activity. Hosts producing itatartaric acid include Aspergillus terreus and Ustilago maydis (Jakubowska et al, 1974; Guevarra and Tabuchi, 1990 a and b; and Geiser et al, 2014). These starting products can then be directed into and engineered pathway such as shown in FIGS. 3, 5, 9, 10, 11, 13 and 15 at the 3HP substrate.

In some embodiments the recombinant microorganism will use a naturally occurring dicarboxylic acid as a substrate for subsequent conversion to a functionalized alpha substituted C4 dicarboxylic acid. For example, the host organism can produce naturally a 3-phosphohydroxypyruvate (see FIG. 9) or itaconic acid (see FIG. 9) or itatartaric acid (see FIG. 9). Hosts producing itatartaric acid include Aspergillus terreus and Ustilago maydis (Jakubowska et al, 1974; Guevarra and Tabuchi, 1990 a and b; and Geiser et al, 2014). These starting products can then be directed into the engineered pathway.
Geiser, Wiebach, Wierckx, and Blank. Prospecting the biodiversity of the fungal family Ustilaginaceae for the production of value-added chemicals. Fulgal Biology and Biotechnology 2014, 1:2.
Guevarra and Tabuchi. Accumulation of Itaconic, 2-hydroxyparaconic, itatartaric, and malic acids by strains of the genus Ustilago. Agric. Biol. Chem. 1990, 54 (9), 2353-2358.
Guevarra and Tabuchi. Production of 2-hydroxyparaconic and itatartaric acids by Ustilago cynodontis and simple recovery process of the acids. Agric. Biol. Chem., 1990, 54 (9), 2359-2365.
Jakubowska and Metodiewa. Studies on the metabolic pathway for itatartaric acid formation by Aspergillus terreus II. Use of (−)-citramalate, citraconate and itaconate by cell-free extracts. Acta Microbiologica Polonica Ser. B 1974, Vol. 6 (23), No. 2, 51-61.

One of ordinary skill in the art will also appreciate that the engineered host cells described herein can include genetic modifications in addition to the enzymes identified in tables provided herein. For instance, one or more enzymes can be attenuated or the activity can be increased in order to optimize the productivity (product concentration/volume/time), yield (product concentration/carbon source fed to organism), or titer (total product produced). The use of various well known techniques such as transcriptomics, metabolomics and the like allow for the further engineering of the recombinant microorganism to force the correct carbon and cofactor flux (ATP, NADH, NADPH, CO₂, O₂ and the like) through the cell to optimize the production.

As mentioned above, the recombinant microorganisms described herein can be derived (the parent strain or host strain) from a microorganism that already produces a significant amount of a desired intermediate. The host strain can be already engineered to produce a significant amount of an intermediate or it can naturally produce a significant amount of the intermediate. For example, the host cell can produce at least 0.1 g/L/hour of an amino acid, a functionalized alpha substituted C4 dicarboxylic acid, a functionalized beta substituted alpha keto acid, a functionalized alpha substituted fumaric acid, an alpha substituted malonyl CoA, 3-hydroxy propionic acid, hydroxymethyl malonic acid, 3-hydroxypropionyl-CoA, hydroxymethyl malonyl-CoA, 2-formyl 3HP-CoA, 2-hydroxymethyl 3H P CoA, hydroxymethyl malonic semialdehyde, 2-(hydroxymethyl) 3HP, alpha-hydroxymethyl acrylyl acid, a functionalized alpha substituted maleic acid, and/or a functionalized alpha substituted malic acid. In other embodiments the host cell can produce 0.5, 0.75, 1.0, 1.5, 2.0 or more g/L/hour of a selected intermediate. Intermediates can include among other things central metabolism products, organic acids, including histidine, arginine, asparagine, lysine, methionine, cysteine, phenylalanine, threonine, glutamate, glutamine, tryptophan, selenocysteine, serine, homoserine, homothreonine, tyrosine, valine, leucine and isoleucine.

In addition to the enzymatic activities depicted in the Figures and specifically identified in the accompanying Tables, the recombinant microorganism may also express an organic acid transporter or premease, such as the Schizosaccharomyces pombe enzyme in U.S. Pat. No. 6,274,311. In addition carbohydrate uptake can be altered through the expression of other transporters.

The host cell into which the various recombinant sequences are introduced can be selected for its tolerance to one or more intermediates or to the products. For example, the host cell can be selected for its ability to produce product in the presence of that product. In some instances the host cell will be tolerant to low pH. The host cell can be either prokaryotic or eukaryotic.

Methods of making the various functionalized alpha substituted C4 dicarboxylic acids or functionalized alpha substituted 3-hydroxypropionic acid are described. These methods include culturing the recombinant microorganism in the presence of a carbohydrate source and separating the functionalized alpha substituted C4 dicarboxylic acid or functionalized alpha substituted 3-hydroxypropionic acid from the fermentation broth.

Methods of making the various functionalized alpha substituted acrylic acids are described. These methods include culturing the recombinant microorganism in the presence of a carbohydrate source and separating the functionalized alpha substituted acrylic acid from the fermentation broth.

The functionalized alpha substituted C4 dicarboxylic acids described herein can be chemically converted through catalytic conversion into functionalized alpha substitute acrylic acids (See FIG. 1) or through enzymatic conversion to functionalized alpha substituted acrylic acids (See FIGS. 11, and 13). The chemical conversions are identified as catalytic steps in FIGS. 3, 9, and 10. Similarly, the functionalized alpha substituted 3HP acids can be chemically converted through a simple dehydration step into a functionalized alpha substitute acrylic acid (See FIG. 5).

The catalytic conversions of the alpha substituted C4 dicarboxylic acids will produce a least a compound of Formula I:

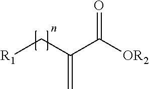

or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and R₂ is individually selected from H and a protecting group, and n is equal to 1 or greater;

the method comprising contacting a metal catalyst with a composition comprising a compound of Formula II, III, or IV:

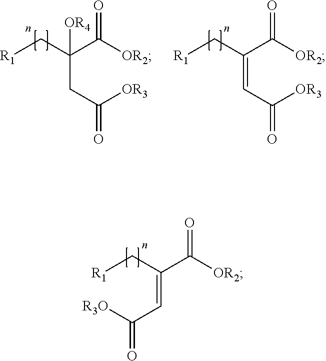

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and protecting groups thereof, and R₂, R₃, R₄ is individually selected from H and a protecting group and n is equal to 1 or greater.

In specific examples, hydroxy functionalized alpha substituted acrylic acids can be formed through catalytic conversion of one or more functionalized alpha substituted C4 dicarboxylic acids. These hydroxy functionalized alpha substituted acrylic acids can have Formula V:

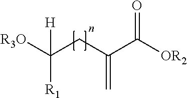

or a salt thereof, wherein: each R₁ is selected from H or CH₃, and R₂ and R₃ are selected from H or a protecting group, and n is equal to 1 or greater. Methods of forming compounds having Formula V include contacting a metal catalyst with composition comprising a compound of Formula VI, VII, VIII:

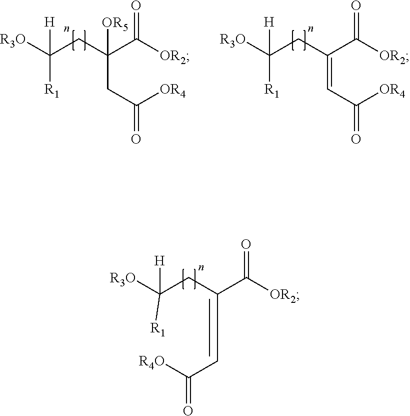

or a salt thereof, wherein: each R₁ is selected from H or CH₃, and R₂, R₃, and R₄ are individually selected from H or a protecting group and n is equal to 1 or greater.

Also described herein are methods of making derivatives of functionalized alpha substituted C4 dicarboxylic acids as shown in Table A below. These methods include contacting a compound selected from formula II, III and IV or a salt, ester or lactone thereof, with a metal catalyst.

TABLE A
Derivatives of functionalized
alpha substituted C4
carboxylic acids

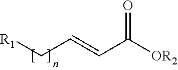

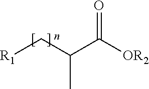

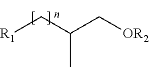

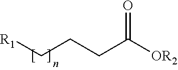

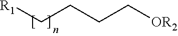

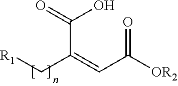

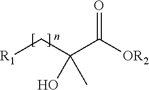

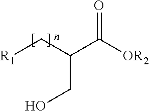

The R groups in the structures shown in Table A are defined as R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and R₂ is individually selected from H and a protecting group, and R₂ is individually selected from H and a protecting group, and n is equal to 1 or greater.

Described herein are methods of making compounds of Formula I

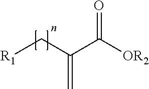

or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, and n is equal to 1 or greater, when n is greater than 1, R₁ can be selected from a carboxylate or methyl; and at least one, at least two, or at least three of the derivatives of a functionalized alpha substituted C4 carboxylic acid shown in Table A. These methods include:
contacting a metal catalyst with a composition comprising a compound of Formula II, III, or IV as shown below respectively,

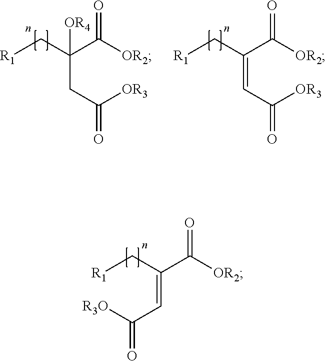

or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂, R₃, R₄ is individually selected from H and a protecting group and n is equal to 1 or greater.

In yet other embodiments, methods are provided that include the formation of a compound of Formula I:

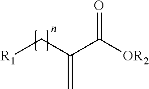

or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate and protecting groups thereof, and n is equal to 1 or greater. These methods include the selective decarboxylation of the beta carboxylate from a functionalized alpha substituted dicarboxylic acid selected from: Formula II, III, or IV, or salts or esters thereof, by contacting such substrate with a catalyst.

In particular embodiments such as those shown in FIG. 9 and described in Examples 2, 3, 4, 5, 6, 7, and 14, methods are taught for making a hydroxyalkyl alpha substituted acrylic acid, or a salt, lactone or ester thereof, the methods include contacting a hydroxyalkyl alpha substituted C4 dicarboxylic acid, or a salt, ester, or lactone thereof, with a metal catalyst.

In yet other particular embodiments, methods are provided for making compounds having Formula I or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, and n is equal to 1 or greater; these methods include contacting a metal catalyst with a composition comprising citric acid, homocitric acid, isopropylmalatic acid or a salt, ester or lactone thereof.

The metal catalysts described herein can be heterogeneous. The metal catalysts can include Ni, Pd, Pt, Cu, Zn, Rh, Ru, Bi, Fe, Co, Os, Ir, V, and mixtures of two or more of these metals. In particular examples the metal catalyst is Cu, Pt or combinations thereof. The metal catalysts can be supported and the can be used in conjunction with a promoter or modifier. The promoter if present can include sulfur.

The step of contacting a substrate with a metal catalyst that is described in the methods taught herein can be done at any temperature that allows for the substrate to be converted to the desired product. For example that contacting step can be done at a temperature of at least about 100° C., or at a temperature of about 100° C. to about 250° C., or at a temperature of about 150° C. to about 200° C. In some instances the catalyst is activated prior to being contacted with the substrate or substrates. Activation can include treating the catalyst with hydrogen gas and it can include activating at elevated temperatures, such as from about 100° C. to about 200° C. In other embodiments the metal catalyst is substantially free of hydrogen.

In one embodiment, the present application relates to a recombinant microorganism comprising a hydroxymethyl malonyl-CoA and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a 3HP CoA-dehydratase (FIG. 16, row E), a CoA transferase (FIG. 16, row F), a carboxylase (FIG. 16, row G), a CoA transferase (FIG. 16, row H), an oxi-reductase (FIG. 16, row I), a reductase (FIG. 16, row J), a dehydratase (FIG. 16, row K), and a coA transferase (FIG. 16, row L). For instance, the recombinant nucleic acid sequence encodes an enzyme selected from a CoA transferase, a CoA carboxylase, CoA transferase, a reductase, a dehydrogenase, a 3HP CoA-dehydratase, a CoA transferase, a carboxylase, a CoA transferase, an oxi-reductase, a reductase, and a CoA transferase. In the alternative, the recombinant nucleic acid sequence encodes an enzyme selected from a CoA transferase, a CoA carboxylase, CoA transferase, a reductase, a dehydrogenase, a carboxylase, a CoA transferase, an oxi-reductase, and a reductase.

In another embodiment, the application relates to a recombinant microorganism comprising an alpha-substituted 3-hydroxypropionic acid and a recombinant nucleic acid sequence encoding an enzyme selected from a CoA transferase, a CoA carboxylase, CoA transferase, a reductase, a dehydrogenase, a carboxylase, a CoA transferase, an oxi-reductase, and a reductase. The application also relates to a recombinant microorganism comprising an alpha-substituted malonyl-CoA and a recombinant nucleic acid sequence encoding an enzyme selected from a CoA transferase, a CoA carboxylase, CoA transferase, a reductase, a dehydrogenase, a carboxylase, a CoA transferase, an oxi-reductase, and a reductase. The application also relates to a recombinant microorganism comprising an alpha-substituted malonic semialdehyde and a recombinant nucleic acid sequence encoding an enzyme selected from a CoA transferase, a CoA carboxylase, CoA transferase, a reductase, a dehydrogenase, a carboxylase, a CoA transferase, an oxi-reductase, and a reductase.

In one embodiment, the microorganism is a prokaryote, for instance, selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus. In another embodiment, the microorganism is a eukaryote, for instance, selected from Candida, Pichia, Saccharomyces, Schizosaccharomyces, Zygosaccharomyces, Kluyveromyces, Debaryomyces, Pichia, Issatchenkia, Yarrowia and Hansenula. Examples of specific host yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis).

In a further embodiment, the application further relates to a method of making an alpha-substituted 3-hydroxypropionic acid of the formula:

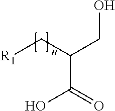

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater;

the method comprising culturing the recombinant microorganism as defined in any one of the foregoing embodiments in the presence of a carbohydrate; and separating the alpha-substituted 3-hydroxypropionic acid or ester or salt thereof.

In a further embodiment, the application further relates to a method for making an alpha-substituted acrylic acid of the formula:

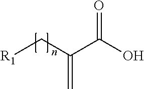

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater;

the method comprising dehydrating an alpha-substituted 3-hydroxypropionic acid to produce the alpha-substituted acrylic acid. For instance, the method further comprises producing the alpha-substituted 3-hydroxypropionic acid by a method defined in the above embodiment.

In other embodiment, the application relates to a compound of the formula:

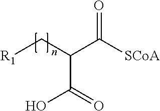

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater; or to a compound of the formula:

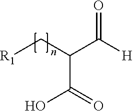

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater, for instance between 1 and 6. In one embodiment, R₁ is hydroxy. In another embodiment, n is 1 or 2. For instance, R₁ is hydroxy and n is 1, or R₁ is hydroxy and n is 2.

One of ordinary skill in the art will appreciate that compositions are also provided herein these compositions include more than one compound, for example a functionalized alpha substituted acrylic acid or a salt, ester or lactone thereof, and one or more compounds selected from an alpha-(hydroxymethyl) malic acid, an alpha-(2-hydroxypropyl) malic acid, an alpha-(1-hydroxyethyl) malic acid and an alpha-(2-hydroxyethyl) malic acid or salts or esters thereof. Other compositions described herein include combinations of a functionalized alpha substituted acrylic acid and one or more derivatives of functionalized alpha substituted C4 carboxylic acids.

More specifically, the present invention relates to a method for producing organic chemicals useful for various applications such as optical materials, paint, reactive diluents, starting materials for surfactants, intermediates for production of pharmaceuticals/agrichemicals, starting materials for resins, co-monomer for methyl acrylate in polymer and the like and the other-substituted acrylate esters obtained thereby.

These and other aspects of the invention will become readily apparent in light of the following detailed description, including the figures and the Examples.

### DESCRIPTION OF THE FIGURES

FIG. 1 shows the general structure of a functionalized (R₁) alpha substituted acrylic acid according to one embodiment, with or without a protecting group (R₂).

FIGS. 2(a)-2(d) show the structure of functionalized alpha substituted organic acids (also referred to as intermediates). FIG. 2(a) shows the structure of functionalized (R₁ with or without n carbon(s)) alpha substituted malic acid with or without protecting group(s) (R₂, R₃, and/or R₄), FIG. 2(b) shows the structure of functionalized (R₁ with or without n carbon(s)) alpha substituted maleic acid with or without protecting group(s) (R₂, and/or R₃); and FIG. 2(c) shows the structure of functionalized (R₁ with or without n carbon(s)) alpha substituted fumaric acid with or without protecting group(s) (R₂, and/or R₃). FIG. 2(d) shows a functionalized alpha substituted 3-hydroxypropionic acid with or without protecting groups (R₂ and/or R₃). The R groups show in FIGS. 2(a)-2(d) are further described in the detailed description.

FIG. 3 shows pathways useful for making functionalized alpha substituted dicarboxylic acids (functionalized intermediates) that can be chemically converted to alpha substituted acrylic acid.

FIG. 4 shows a table providing exemplary enzymes that can be included in recombinant cells, including prokaryotic and eukaryotic cells, that are engineered to produce functionalized alpha substituted dicarboxylic acids and functionalized alpha substituted 3-hydroxypropionic acids and salts and esters thereof, for instance, enzymes which can be used in the pathway illustrated in FIG. 3.

FIG. 5 shows pathways useful for making alpha substituted 3-hydroxypropionic acid. Such functionalized alpha substituted 3-hydroxypropionic acid can be used among other things as a substrate for chemical conversion to an acrylate.

FIG. 6 shows a table listing exemplary enzymes that can be included in recombinant cells, including prokaryotic and eukaryotic cells that are engineered to produce functionalized alpha substituted 3-hydroxypropionic acid, for instance, though the pathway illustrated in FIG. 5. In some instances, the functionalized alpha substituted 3-hydroxypropionic acid can be chemically converted to alpha substituted acrylic acid.

FIG. 7 shows the variety of potential functionalized groups that can be engineered to be included in the functionalized alpha substituted dicarboxylic acids, functionalized (R¹) alpha substituted 3-hydroxypropionic acids and that therefore can be included in the functionalized alpha substituted acrylates. One of ordinary skill in the art will appreciate that any acrylate resulting from the use of an amino acid in the pathways shown in FIGS. 3 and 5 will be an acrylate wherein the methylene group is in the same position as the amino group in the initial amino acid residue. In pathways that are designed to include one or more spacer carbons, the spacer carbons will be inserted between the methylene group and the original amino acid side chain.

FIG. 8 shows a table which provides nomenclature used to describe various compounds in the detailed description. One of ordinary skill in the art will appreciate that a single compound can be identified by a variety of names ranging from an IUPAC name to a common name.

FIG. 9 shows exemplary pathways for making hydroxymethyl substituted C4 dicarboxylic acids and hydroxyethyl substituted C4 dicarboxylic acids. These products can be converted chemically to hydroxyalkyl substituted acrylic acids (e.g. HMA and HEA) which in turn can be converted either enzymatically or chemically into tulipalin. See also Examples 2, 3, 4, 5, 6, 7, and 14.

FIG. 10 shows exemplary pathways for making methyl 2-methylene 4 butyrolactone (MeMBL). The specific enzymes identified in FIG. 10 are further described in Examples 8 and 15. The letter used to identify specific enzymatic steps corresponds to the enzymes identified in the table of FIG. 4.

FIG. 11 shows fully biological routes to make the functionalized alpha substituted acrylic acids and functionalized intermediates. These fully biological routes can be identified by the inclusion of the catalytic steps designated with an “I”. The inclusion of a fully biological route to the functionalized alpha substituted acrylic acid also allows for fully biological routes to cyclized product and esters.

FIG. 12 shows a table which is substantially similar to the table of FIG. 4, however, the table of FIG. 12 includes specific enzymes useful for the fully biological pathways shown in FIG. 11.

FIG. 13 shows fully biological routes to alpha substituted acrylic acids using an alpha substituted 3HP intermediate. This figure is similar to that shown in FIG. 5, except that it provides for a fully biological route to the product.

FIG. 14 shows a table which includes specific enzymes that can be used to make recombinant microorganisms to produce the functionalized alpha substituted acrylic acids from the alpha substituted 3HP intermediate as described in FIG. 13.

FIG. 15 shows a fully or partly biological route to making alpha hydroxymethyl acrylic acid or an intermediate thereof through an alpha-substituted malonyl CoA pathway.

FIG. 16 shows a table which includes specific enzymes that can be used to make recombinant microorganisms to produce the functionalized alpha substituted acrylic acids from 3HP using the routes illustrated in FIG. 15 or FIG. 19.

FIG. 17 shows a fully biological route to alpha hydroxyethyl acrylic acid using the itaconic acid intermediate. The alpha hydroxyethyl acrylic acid can be converted to the lactone form, tulipalin A, by an enzyme having the activity described in the table of FIG. 18, row C.

FIG. 18 shows a table which includes specific enzymes that can be used to make recombinant microorganisms to produce the alpha hydroxyethyl acrylic acid and/or tulipalin A from itaconic acid, for instance, through the route illustrated in FIG. 17.

FIG. 19 shows a fully or partly biological route to making alpha-substituted acrylic acids or an intermediate thereof similarly to the pathway illustrated in FIG. 15.

FIG. 20 shows synthase activity results from experiments described in Example 2: (a) synthase activities of various candidate cells include a synthase gene using hydroxypyruvate as substrate compared to cells containing an empty vector; (b) comparative results of wild type and mutant synthase activities with hydroxypyruvate.

FIG. 21 shows hydroxyparaconic acid production in: (a) U. maydis at pH 3, pH 5, and pH 7; and (b) Aspergillus terreus over time at pH 3 (see also Example 4).

FIG. 22 shows activity of dehydratase candidates with itatartaric acid (ITT), where, within each grouping, the right bar represents “no substrate” and the left bar represents ITT: (a) levels of ITT dehydrated product as compared to the no substrate control in cells expressing dehydratase candidates (lysate incubated with or without itatartaric acid (ITT) present overnight at 30° C., samples analyzed by HPLC); and (b) NMR results of ITT standard and lysate containing the dehydrated ITT product (top 2 lines), and NMR results of analogs of ITT and their dehydrated product, citramalic→citraconic, citric→cis-aconitic, malic→maleic, homocitric→homoaconitic—top bar indicating the region of characteristic peaks of dehydration products (see also Example 5).

FIG. 23 shows levels of dehydratase product as compared to the no substrate control in E coli cells with either acnA or acnB deleted, expressing either empty vector (ptrc) or plasmids expressing endogenous E coli dehydratases, acnA or acnB (see also Example 5, indicated lysate incubated with or without itatartaric acid (ITT) overnight at 30° C., samples analyzed by HPLC).

FIG. 24 shows a GC/MS chromatogram of results produced by Sodium homocitrate catalyzed by Pt/Al₂O₃ (see also Example 12)

FIGS. 25 (a) and (b) shows mass spectra of the 2-methylglutaric acid peak having a retention time of 6.68 minutes in the chromatogram of FIG. 24.

FIG. 26 shows a mass spectrum of the 1,2,4-butane tricarboxylic acid peak having a retention time of 9.612 minutes in the chromatogram of FIG. 24.

FIG. 27 shows (a) a GC-MS chromatogram of 2-methylene glutaric acid from Example 12 from a reaction catalyzed by a Cu-based catalyst and sodium homocitrate; (b) and (c) mass spectra of 2-methylene glutaric acid obtained with Cu-based catalyst and sodium homocitrate.

FIG. 28 shows results obtained in Example 13 from the reaction of 2-isopropylmalic acid with Cu-based catalyst under H₂ (reaction conditions: 180° C., metal Cu (50 wt %), 16 h, 450 psi H₂, substrate (0.121 mmol), H₂O), showing (a) a GC/MS chromatogram; and (b) and (c) mass spectra of butanoic acid 2,3-dimethyl, methyl ester with a retention time of 3.901 mins.

FIG. 29 shows results obtained in Example 13 from the reaction of 2-isopropylmalic acid with Cu catalyst in H₂O (reaction conditions: 2-isopropylmalic acid (0.12 mmol) Cu #2 catalyst (Cu-0860, supplied as oxide, BASF); 180° C. 16 h, H₂O (1 mL), N₂ (450 psi)), showing (a) a GC/MS chromatogram of the reaction product; (b) a GC/MS chromatogram of 2-isopropylacrylic acid (methyl ester derivative); and (c) a mass spectrum of 2-isopropylacrylic acid (methyl ester derivative) peak having a retention time of 3.967 min (in (b)).

FIG. 30 shows (a) a GC-MS chromatogram and (b) a mass spectrum of 2-isopropyl malic acid (methyl ester derivative) having a retention time of 7.329 min (see also Example 13).

FIG. 31 shows (a) a GC-MS chromatogram and (b) a mass spectrum of 4-methyl-2-pentenoic acid (authentic sample after derivatization) (see also Example 13).

FIGS. 32 (a) and (b) show mass spectra of 4-methyl-2-pentanoic acid from reaction mixture after methyl ester derivatization; and (c) a mass spectrum of 2-(1-methylethyl)-2-butenedioic acid methyl ester derivative, peak having a retention time of 6.975 min (see also Example 13).

FIG. 33(a) presents comparative tolerance data of potential host organisms to 3-hydroxymethyl 3-hydroxypropionic acid (HM3HP) where Km=K. marxianus, Sc=S. cerevisiae, Ec=E. coli (from Example 17). Within each grouping, the right bar represents Ec, the left bar represents Km, and the middle bar represents Sc. FIG. 33(b) shows results obtained using the PCC pyruvate accumulation coupled assay from Example 21.

### DETAILED DESCRIPTION

### Definitions

General methods for molecular biology procedures and recipes for buffers, solutions, and media in the following examples are described in J. Sambrook, and D. W. Russell, Molecular Cloning: A Laboratory Manual, 3rd Ed., Cold Spring Harbor Laboratory Press, Cold Spring Harbor, N.Y., 2001. When listed, instructions from individual manufactures were used for some of the procedures. Restriction enzymes were purchased from New England Biolabs (Ipswich, Mass.), unless otherwise stated, and used in appropriate buffers as suggested by the manufacture. All chemicals were purchased from Sigma Aldrich (St. Louis, Mo.), unless otherwise specified.

For purposes of this application, “native” as used herein with regard to a metabolic pathway refers to a metabolic pathway that exists and is active in the wild-type host strain. Genetic material such as coding regions, genes, promoters and terminators is “native” for purposes of this application if the genetic material has a sequence identical to (apart from individual-to-individual mutations which do not affect function) a genetic component that is present in the genome of the wild-type host cell (i.e., the exogenous genetic component is identical to an endogenous genetic component).

For purposes of this description, genetic material such as a coding region, a gene, a promoter and a terminator is “endogenous” to a cell if it is (i) native to the cell, (ii) present at the same location as that genetic material is present in the wild-type cell and (iii) under the regulatory control of its native promoter and its native terminator and (iv) has not been altered directly or through a directed selection process.

For purposes of this application, genetic material such as coding sequence, genes, promoters and terminators are “exogenous” to a cell if they are (i) non-native to the cell and/or (ii) are native to the cell, but are present at a location different than where that genetic material is present in the wild-type cell and/or (iii) are under the regulatory control of a non-native promoter and/or non-native terminator. Extra copies of native genetic material are considered as “exogenous” for purposes of this description, even if such extra copies are present at the same locus as that genetic material is present in the wild-type host strain and/or (iv) they are altered directly or through a selection process.

As used herein, the term “control sequences” included enhancer sequences, terminator sequences and promoters. As used herein “promoter” refers to an untranslated sequence located upstream (i.e., 5′) to the translation start codon of a gene (generally a sequence of about 1 to 1500 base pairs (bp), preferably about 100 to 1000 bp and especially of about 200 to 1000 bp) which controls the start of transcription of the gene. Where the promoters are non-native, they may be identical to or share a high degree of sequence identity (i.e., at least about 80%, at least about 85%, at least about 90%, at least about 95%, or at least about 99%) with one or more native promoters. Other suitable promoters and terminators include those described, for example, in WO99/14335, WO00/71738, WO02/42471, WO03/102201, WO03/102152 and WO03/049525.

The term “terminator” as used herein refers to an untranslated sequence located downstream (i.e., 3′) to the translation termination codon of a gene (generally a sequence of about 1 to 1500 bp, preferably of about 100 to 1000 bp, and especially of about 200 to 500 bp) which controls the end of transcription of the gene. Examples of terminators that may be linked to one or more exogenous genes in the yeast cells provided herein include, but are not limited to, terminators for PDC1, XR, XDH, transaldolase (TAL), transketolase (TKL), ribose 5-phosphate ketol-isomerase (RKI), CYB2, or iso-2-cytochrome c (CYC) genes or the galactose family of genes (especially the GAL 10 terminator), as well as any of those described in the various Examples that follow. Where the terminators are non-native, they may be identical to or share a high degree of sequence identity (i.e., at least about 80%, at least about 85%, at least about 90%, at least about 95%, or at least about 99%) with one or more native terminators.

A promoter or terminator is “operatively linked” to a coding sequence if its position in the genome relative to that of the coding sequence is such that the promoter or terminator, as the case may be, performs its transcriptional control function. One of ordinary skill in the art will also appreciate that the DNA sequence can include regions that give rise to RNA sequences that modulate translation.

“Increasing or decreasing” activity with regard to enzyme activities refers to the activity either being greater than that enzymatic activity found in the wild type strain (increasing activity), or refers to the activity being less than that enzymatic activity found in the wild type strain (decreasing activity or otherwise referred to as attenuating). One ordinarily skilled in the art will appreciate that the modulation of activity can be accomplished by (i) controlling polypeptide: polypeptide interactions, (ii) polypeptide: metabolite interactions (feedback inhibition), (iii) polypeptide/nucleic acid interactions, (iv) modifying the amino acid sequence to increase enzymatic activity and (v) nucleic acid interactions.

“Deletion or disruption” with regard to a gene means that either the entire coding region of the gene is eliminated (deletion) or the coding region of the gene, its promoter, and/or its terminator region is modified (such as by deletion, insertion, or mutation) such that the gene no longer produces an active enzyme, produces a severely reduced quantity of enzyme (at least 75% reduction, preferably at least 85% reduction, more preferably at least 95% reduction), or produces an enzyme with severely reduced (at least 75% reduced, preferably at least 85% reduced, more preferably at least 95% reduced) activity. A deletion or disruption of a gene can be accomplished by, for example, forced evolution, mutagenesis or genetic engineering methods, followed by appropriate selection or screening to identify the desired mutants.

“Overexpress” means the artificial expression of an enzyme in increased quantity. Overexpression of an enzyme may result from the presence of one or more exogenous gene(s), genetic engineering to increase the expression of the endogenous gene, or from other conditions. For purposes of this invention, a yeast cell containing at least one exogenous gene is considered to overexpress the enzyme(s) encoded by such exogenous gene(s).

A “recombinant microorganism” is a microorganism, either eukaryotic or prokaryotic, that has a nucleotide sequence that has been altered by human intervention to include a sequence that is not the same as that found in the progenitor microorganism. One of ordinary skill the art will appreciate that such nucleic acid sequence alterations can be introduced through a variety of methods, including for example, mutation and selection, transformation, mating, homologous recombination and the like. Any method known in the art can be used to generate such recombinant microorganism. Moreover, the nucleic acid sequence alteration can be chromosomal or extrachromosomal.

A recombinant eukaryotic cell can be a yeast or a fungal cell comprising certain genetic modifications. The host yeast or fungi cell is one which as a wild-type strain is natively capable of metabolizing at least one sugar to pyruvate. Suitable host yeast cells include (but are not limited to) yeast cells classified under the genera Candida, Pichia, Saccharomyces, Schizosaccharomyces, Zygosaccharomyces, Kluyveromyces, Debaryomyces, Pichia, Issatchenkia, Yarrowia and Hansenula. Examples of specific host yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis). Suitable strains of K. marxianus and C. sonorensis include those described in WO 00/71738 A1, WO 02/42471 A2, WO 03/049525 A2, WO 03/102152 A2 and WO 03/102201A2. Suitable strains of I. orientalis are ATCC strain 32196 and ATCC strain PTA-6648. In addition, fungi may include Aspergillus niger, Aspergillus terreus, Aspergillus oryzae, Ustilago maydis, Ustilago cynodontis, or other fungi.

In some embodiments of the invention the host cell is Crabtree negative as a wild-type strain. The Crabtree effect is defined as the occurrence of fermentative metabolism under aerobic conditions due to the inhibition of oxygen consumption by a microorganism when cultured at high specific growth rates (long-term effect) or in the presence of high concentrations of glucose (short-term effect). Crabtree negative phenotypes do not exhibit this effect, and are thus are able to consume oxygen even in the presence of high concentrations of glucose or at high growth rates.

Modifications (insertion, deletions and/or disruptions) to the genome of the host cell described herein can be performed using methods known in the art. Exogenous genes may be integrated into the genome in a targeted or a random manner using, for example, well known electroporation and chemical methods (including calcium chloride and/or lithium acetate methods). In those embodiments where an exogenous gene is integrated in a targeted manner, it may be integrated into the locus for a particular native gene, such that integration of the exogenous gene is coupled with deletion or disruption of a native gene. Alternatively, the exogenous gene may be integrated into a portion of the native genome that does not correspond to a gene. Methods for transforming a yeast cell with an exogenous construct are described in, for example, WO99/14335, WO00/71738, WO02/42471, WO03/102201, WO03/102152, WO03/049525, WO2007/061590, WO 2009/065778 and PCT/US2011/022612. Insertion of exogenous genes is generally performed by transforming the cell with one or more integration constructs or fragments. The terms “construct” and “fragment” are used interchangeably herein to refer to a DNA sequence that is used to transform a cell. The construct or fragment may be, for example, a circular plasmid or vector, a portion of a circular plasmid or vector (such as a restriction enzyme digestion product), a linearized plasmid or vector, or a PCR product prepared using a plasmid or genomic DNA as a template. An integration construct can be assembled using two cloned target DNA sequences from an insertion site target. The two target DNA sequences may be contiguous or non-contiguous in the native host genome. In this context, “non-contiguous” means that the DNA sequences are not immediately adjacent to one another in the native genome, but instead are separated by a region that is to be deleted. “Contiguous” sequences as used herein are directly adjacent to one another in the native genome. Where targeted integration is to be coupled with deletion or disruption of a target gene, the integration construct also functions as a deletion construct. In such an integration/deletion construct, one of the target sequences may include a region 5′ to the promoter of the target gene, all or a portion of the promoter region, all or a portion of the target gene coding sequence, or some combination thereof. The other target sequence may include a region 3′ to the terminator of the target gene, all or a portion of the terminator region, and/or all or a portion of the target gene coding sequence. Where targeted integration is not to be coupled to deletion or disruption of a native gene, the target sequences are selected such that insertion of an intervening sequence will not disrupt native gene expression. An integration or deletion construct is prepared such that the two target sequences are oriented in the same direction in relation to one another as they natively appear in the genome of the host cell. The gene expression cassette is cloned into the construct between the two target gene sequences to allow for expression of the exogenous gene. The gene expression cassette contains the exogenous gene, and may further include one or more regulatory sequences such as promoters or terminators operatively linked to the exogenous gene.

It is usually desirable that the deletion construct may also include a functional selection marker cassette. When a single deletion construct is used, the marker cassette resides on the vector downstream (i.e., in the 3′ direction) of the 5′ sequence from the target locus and upstream (i.e., in the 5′ direction) of the 3′ sequence from the target locus. Successful transformants will contain the selection marker cassette, which imparts to the successfully transformed cell some characteristic that provides a basis for selection.

A cell is considered to be “resistant” to a compound if it is capable of remaining viable in the presence of the substance. In some instances a resistant cell may be capable of growth and multiplication in the presence of the compound. For example, a host cell, such as a recombinant microorganism that is engineered to produce one or more functionalized alpha substituted C4 dicarboxylic acids is resistant to the functionalized alpha substituted C4 dicarboxylic acid if it remains viable in the presence of the functionalized alpha substituted C4 dicarboxylic acid. For example, a recombinant microorganism is resistant to a functionalized alpha substituted C4 dicarboxylic acid if it remains viable in the presence of media containing at least 1%, 3%, 5%, 6%, 7%, 8%, 9% or 10% of the functionalized alpha substituted C4 dicarboxylic acid. Test methods for determining a microorganism's resistance to compounds are well known in the art, for example the test method described in Example 1A of WO 2012/103261 and/or Example 1 provided below can be used.

Similarly, a host cell, such as a recombinant microorganism that is engineered to produce one or more functionalized alpha substituted acrylic acids is resistant to the functionalized alpha substituted acrylic acids if it remains viable in the presence of the functionalized alpha substituted acrylic acids. For example, a recombinant microorganism is resistant to a functionalized alpha substituted acrylic acid if it remains viable in the presence of media containing at least 1%, 3%, 5%, 6%, 7%, 8%, 9% or 10% of the functionalized alpha substituted acrylic acid. Test methods for determining a microorganism's resistance to compounds are well known in the art, for example the test method described in Example 1A of WO 2012/103261 and/or Example 17 provided below can be used.

A “selection marker gene” may encode for a protein needed for the survival and/or growth of the transformed cell in a selective culture medium. Typical selection marker genes encode proteins that (a) confer resistance to antibiotics or other toxins, (such as, for example, zeocin (Streptoalloteichus hindustanus ble bleomycin resistance gene), G418 (kanamycin-resistance gene of Tn903) or hygromycin (aminoglycoside antibiotic resistance gene from E. coli)), (b) complement auxotrophic deficiencies of the cell (such as, for example, amino acid leucine deficiency (K. marxianus LEU 2 gene) or uracil deficiency {e.g., K. marxianus or S. cerevisiae URA3 gene)); (c) enable the cell to synthesize critical nutrients not available from simple media, or (d) confer ability for the cell to grow on a particular carbon source, (such as a MELS gene from S. cerevisiae, which encodes the alpha-galactosidase (melibiase) enzyme and confers the ability to grow on melibiose as the sole carbon source). Preferred selection markers include the zeocin resistance gene, G418 resistance gene, a MELS gene, a URA3 gene and hygromycin resistance gene. Another preferred selection marker is an L-lactate:ferricytochrome c oxidoreductase (CYB2) gene cassette, provided that the host cell either natively lacks such a gene or that its native CYB2 gene(s) are first deleted or disrupted.

The construct may be designed so that the selection marker cassette can become spontaneously deleted as a result of a subsequent homologous recombination event. A convenient way of accomplishing this is to design the vector such that the selection marker gene cassette is flanked by direct repeat sequences. Direct repeat sequences are identical DNA sequences, native or not native to the host cell, and oriented on the construct in the same direction with respect to each other. The direct repeat sequences are advantageously about 50-1500 bp in length. It is not necessary that the direct repeat sequences encode for anything. This construct permits a homologous recombination event to occur. This event occurs with some low frequency, resulting in cells containing a deletion of the selection marker gene and one of the direct repeat sequences. It may be necessary to grow transformants for several rounds on nonselective or selective media to allow for the spontaneous homologous recombination to occur in some of the cells. Cells in which the selection marker gene has become spontaneously deleted can be selected or screened on the basis of their loss of the selection characteristic imparted by the selection marker gene, or by using PCR or Southern Analysis methods to confirm the loss of the selection marker.

In some embodiments, an exogenous gene may be inserted using DNA from two or more integration fragments, rather than a single fragment. In these embodiments, the 3′ end of one integration fragment contains a region of homology with the 5′ end of another integration fragment. One of the fragments will contain a first region of homology to the target locus and the other fragment will contain a second region of homology to the target locus. The gene cassette to be inserted can reside on either fragment, or be divided among the fragments, with a region of homology at the 3′ and 5′ ends of the respective fragments, so the entire, functional gene cassette is produced upon a crossover event. The cell is transformed with these fragments simultaneously. A selection marker may reside on any one of the fragments or may be divided between the fragments with a region of homology as described. In other embodiments, transformation from three or more constructs can be used in an analogous way to integrate exogenous genetic material.

Deletions and/or disruptions of native genes can be performed by transformation methods, by mutagenesis and/or by forced evolution methods. In mutagenesis methods cells are exposed to ultraviolet radiation or a mutagenic substance, under conditions sufficient to achieve a high kill rate (60-99.9%, preferably 90-99.9%) of the cells. Surviving cells are then plated and selected or screened for cells having the deleted or disrupted metabolic activity. Disruption or deletion of the desired native gene(s) can be confirmed through PCR or Southern analysis methods.

Cells of the invention can be cultivated to produce intermediates, functionalized alpha substituted C4 dicarboxylic acids, and/or functionalized alpha substituted acrylic acids and corresponding ester or lactone thereof, either in the free acid form or in salt form (or both). The recombinant cell is cultured in a medium that includes at least one carbon source that can be fermented by the cell. Examples include, but are not limited to, twelve carbon sugars such as sucrose, hexose sugars such as glucose or fructose, glycan, starch, or other polymer of glucose, glucose oligomers such as maltose, maltotriose and isomaltotriose, panose, and fructose oligomers, and pentose sugars such as xylose, xylan, other oligomers of xylose, or arabinose.

The medium will typically contain, in addition to the carbon source, nutrients as required by the particular cell, including a source of nitrogen (such as amino acids, proteins, inorganic nitrogen sources such as ammonia or ammonium salts, and the like), and various vitamins, minerals and the like. In some embodiments, the cells of the invention can be cultured in a chemically defined medium.

Other cultivation conditions, such as temperature, cell density, selection of substrate(s), selection of nutrients, and the like are not considered to be critical to the invention and are generally selected to provide an economical process. Temperatures during each of the growth phase and the production phase may range from above the freezing temperature of the medium to about 50° C., although this depends to some extent on the ability of the strain to tolerate elevated temperatures. A preferred temperature, particularly during the production phase, is about 27 to 45° C.

During cultivation, aeration and agitation conditions may be selected to produce a desired oxygen uptake rate. The cultivation may be conducted aerobically, microaerobically, or anaerobically, depending on pathway requirements. In some embodiments, the cultivation conditions are selected to produce an oxygen uptake rate of around 2-25 mmol/L/hr, preferably from around 5-20 mmol/L/hr, and more preferably from around 8-15 mmol/L/hr. “Oxygen uptake rate” or “OUR” as used herein refers to the volumetric rate at which oxygen is consumed during the fermentation. Inlet and outlet oxygen concentrations can be measured with exhaust gas analysis, for example by mass spectrometers. OUR can be calculated using the Direct Method described in Bioreaction Engineering Principles 2nd Edition, 2003, Kluwer Academic/Plenum Publishers, p. 449, equation I.

The cultivation may be continued until a yield of desired product on the carbon source is, for example, at least 10%, at least 20%, at least 30%, at least 40%, at least 50%, at least 60%, at least 70% or greater than 70% of the theoretical yield. The yield of product can be at least 80% or at least 90% of the theoretical yield. The concentration, or titer, of product produced in the cultivation will be a function of the yield as well as the starting concentration of the carbon source. In certain embodiments, the titer may reach at least 1, at least 3, at least 5, at least 10, at least 20, at least 30, at least 40, at least 50, or greater than 50 g/L at some point during the fermentation, and preferably at the end of the fermentation.

The term “convert” refers to the use of either chemical means or polypeptides in a reaction which changes a first intermediate to a second intermediate. The term “chemical conversion” refers to reactions that are not actively facilitated by polypeptides. The term “biological conversion” refers to reactions that are actively facilitated by polypeptides. Conversions can take place in vivo or in vitro. When biological conversions are used the polypeptides and/or cells can be immobilized on supports such as by chemical attachment on polymer supports. The conversion can be accomplished using any reactor known to one of ordinary skill in the art, for example in a batch or a continuous reactor.

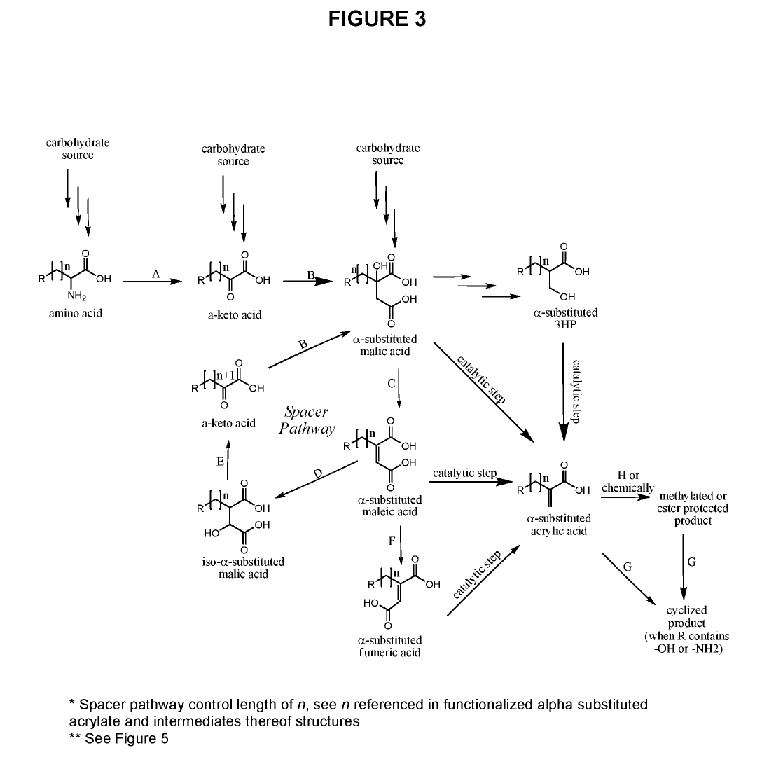

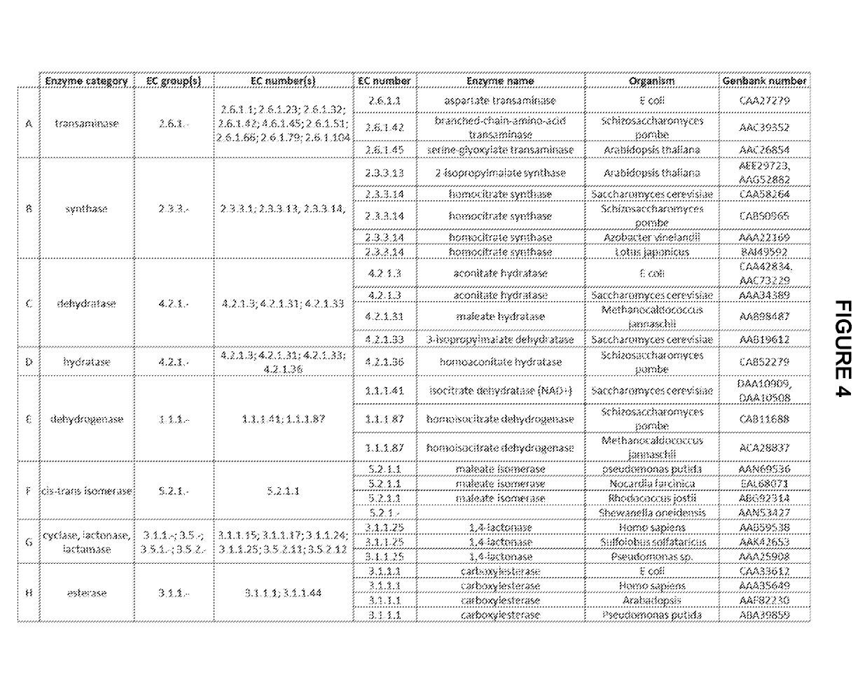

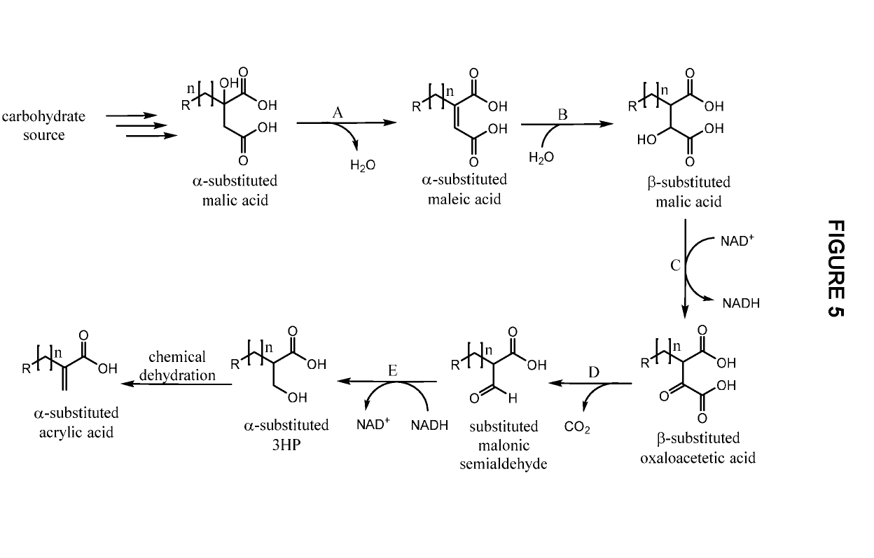

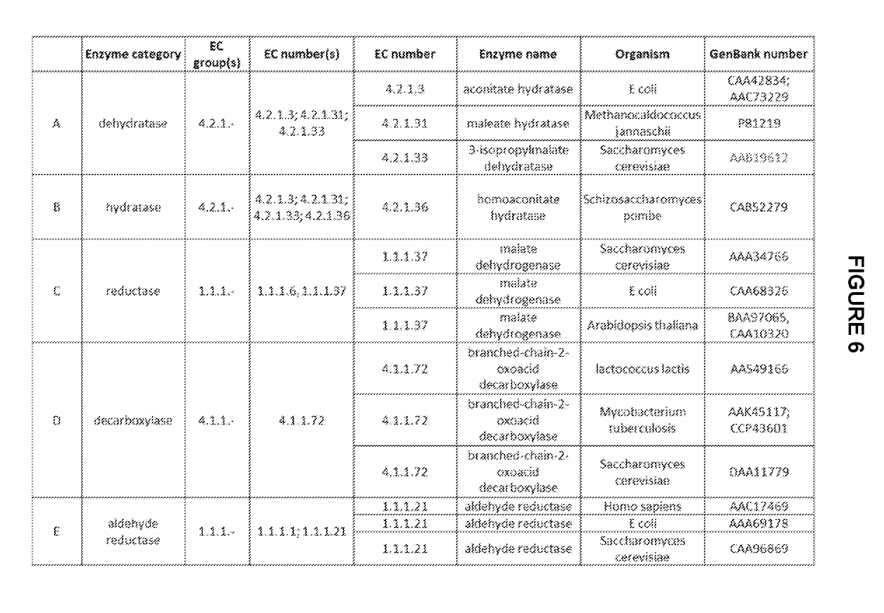

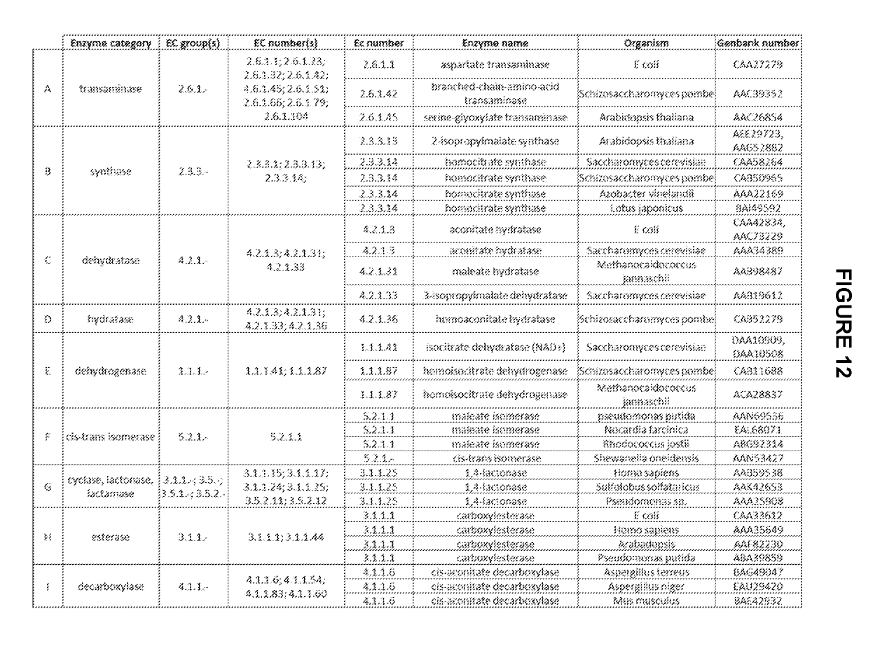

Methods are also provided that include contacting a first polypeptide with a substrate and making a first product, and then contacting the first product created with a second polypeptide and creating a second product, and then contacting the second product created with a third polypeptide and creating a third product etc. The polypeptides used to convert an intermediate to the next product or next intermediate in a pathway are described in FIG. 4 (describing enzymes that can be used for the indicated conversions in FIGS. 3, 9 and 10) and FIG. 6 (describing enzymes that can be used for the indicated conversions in FIG. 5) and FIGS. 12, 14, 16, and 18.

The term “salt” includes any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary anions include, but are not limited to: chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluoromethylbenzenesulfonate, hydroxide, aluminates and borates. Exemplary cations include, but are not limited to: monovalent alkali metal cations, such as lithium, sodium, potassium, and cesium, and divalent alkaline earth metals, such as beryllium, magnesium, calcium, strontium, and barium. Also included are transition metal cations, such as gold, silver, copper and zinc, as well as non-metal cations, such as ammonium salts. One of ordinary skill in the art will appreciate that when fully biological routes are used to produce compounds the compound will be substantially in acid form or in salt form depending upon the pKa of the compound and the pH of the media.

An “ester” as used herein includes, as nonlimiting examples, methyl esters, ethyl esters, and isopropyl esters, and esters which result from the addition of a protecting group on a corresponding carboxyl moiety.

A “lactone” as used herein refers to the cyclic ester compounds which result from the condensation of an alcohol group and a carboxylic acid group on the compounds provided herein. A nonlimiting example is the lactone which results from the condensation of homocitric acid, or its salts (ie. homocitric acid lactone).

As used herein, chemical structures which contain one or more stereocenters depicted with bold and dashed bonds (i.e., [symbol] [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

Compounds, as described herein, can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric isomers, tautomers, and isotopes of the structures depicted. Compounds herein identified by name or structure as one particular tautomeric form are intended to include other tautomeric forms unless otherwise specified. All compounds, salts, esters, and lactones thereof, can be found together with other substances such as water and solvents (e.g. hydrates and solvates).

In some embodiments, the compounds described herein, or salts, esters, or lactones thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

It is appreciated that certain features of the disclosure, which are, for clarity, described in the context of separate embodiments, can also be provided in combination in a single embodiment. Conversely, various features of the invention which are, for brevity, described in the context of a single embodiment, can also be provided separately or in any suitable subcombination.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

I. Engineered Pathways

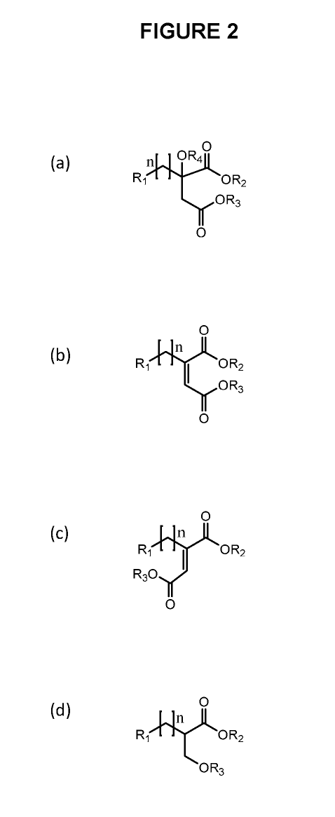

The recombinant microorganisms described herein display enzyme activities that cause them to be capable of making a non-natural amount of functionalized alpha substituted C4 dicarboxylic acids as shown in FIG. 2. In some instances the recombinant microorganism produces more than one type of alpha substituted dicarboxylic acid, such as those shown in FIGS. 2(a), 2(b), and 2(c). The phrase “non-natural” amount refers to the fact that the recombinant microorganisms described herein produce a higher concentration of the alpha substituted C4 dicarboxylic acid as compared to the starting host cell that was used as the starting point for introducing the recombinant nucleic acid sequences.

The recombinant microorganisms described herein display enzyme activities that cause them to be capable of making a non-natural amount of functionalized alpha substituted acrylic acid/or salt and corresponding ester or lactone, thereof. In some instances the recombinant microorganism produces more than one type of alpha substituted acrylic acid. The phrase “non-natural” amount refers to the fact that the recombinant microorganisms described herein produce a higher concentration of the alpha substituted acrylic acid as compared to the starting host cell that was used as the starting point for introducing the recombinant nucleic acid sequences.

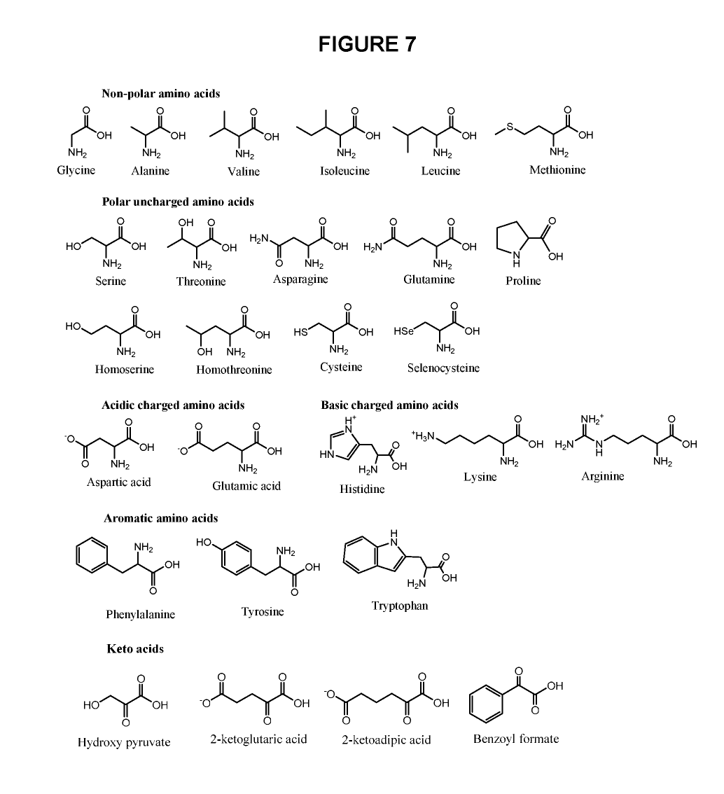

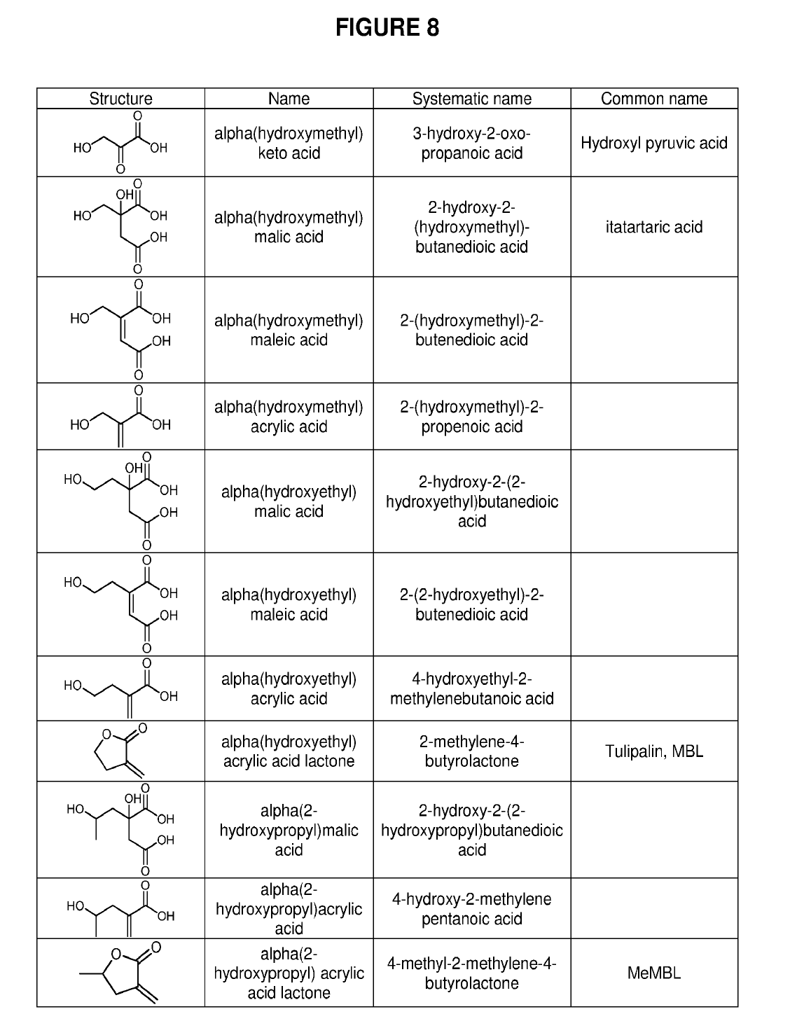

The term “functionalized alpha substituted C4 dicarboxylic acid” as it is used herein refers to the fact that the carbon that is alpha to a carboxylic acid in the alpha functionalized dicarboxylic acid comprises at least four bonds to non-hydrogen atoms. For example, with reference to FIG. 2(a) an alpha functionalized malic acid is shown and the alpha carbon from the carboxylic acid comprises the following bonds, —COOH, —CH₂—, —OR₄, and -[n]R₁. In contrast a non-functionalized malic acid would have an alpha carbon that comprises the following bonds, —COOH, —CH₂—, —H, and —OH. The functionalized group, the -[n]R₁, can be any group selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and those shown in FIGS. 7 and 8. In another example, with reference to FIGS. 2(b) and 2(c) an alpha functionalized maleic acid (cis) or alpha functionalized fumaric acid (trans), respectively, are shown and the alpha carbon from the carboxylic acid comprises the following bonds, —COOH, ═CH—, and -[n]R₁. In contrast a non-functionalized maleic acid or fumaric acid would have an alpha carbon that comprises the following bonds, —COOH, ═CH—, and —H. The functionalized group, the -[n]R₁, can be any group selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and those shown in FIGS. 7 and 8. One of ordinary skill in the art will appreciate that references to an organic acid are understood to include the acid, as well as its salt or corresponding esters and protecting groups unless contest clearly indicates otherwise. For example FIGS. 2(a), 2(b), and 2(c) use R₂ and R₃ to indicate these possibilities. Moreover, one of ordinary skill in the art will appreciate that more generally when a hydroxyl is present reference to the alcohol also includes a reference to the alcohol with a protecting group.

The term “functionalized alpha substituted 3-hydroxypropionic acid (3HP)” as it is used herein refers to the fact that the carbon that is alpha to the carboxylic acid in the functionalized alpha substituted 3HP comprises at least three bonds to non-hydrogen atoms. For example, with reference to FIG. 2(d) an functionalized alpha substituted 3HP is shown and the alpha carbon from the carboxylic acid comprises the following bonds, —COOH, —CH₂OH, —H and -[n]R₁. In contrast a non-functionalized 3HP would have an alpha carbon that comprises the following bonds, —COOH, —CH₂OH, —H, and —H. The functionalized group, the -[n]R₁, can be any group selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and those shown in FIGS. 7 and 8. Similar to what is stated above, references to an organic acid are understood to include the acid, as well as its salt or corresponding esters and protecting groups unless contest clearly indicates otherwise. In the instance of functionalized alpha substituted 3HP, reference to the hydroxyl group on the third carbon shall also be understood to include instances were a protecting group is present.

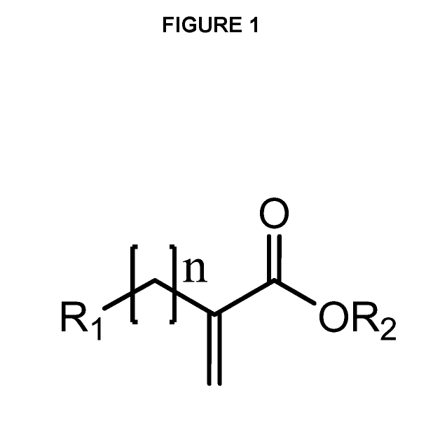

The term “functionalized alpha-substituted acrylic acid” as it is used herein refers to the fact that the carbon that is alpha to the methylene group in the functionalized alpha-substituted acrylic acid comprises four (4) bonds to non-hydrogen atoms. For example, with reference to FIG. 1 a functionalized alpha substituted acrylate is shown and the alpha carbon from the methylene group comprises the following bonds, —COOH, ═CH₂, and -[n]R₁. In contrast a non-functionalized alpha substituted acrylic acid would have an alpha carbon that comprises the following bonds, COOH, ═CH₂, and —H. The functionalized group, the -[n]R₁, can be any group selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and those shown in FIGS. 7 and 8. Similar to what is stated above, references to an organic acid are understood to include the acid, as well as its salt or corresponding esters and protecting groups unless contest clearly indicates otherwise. As is apparent from FIG. 1, R₂ can be a protecting group and in the instance where R₁ comprises an acidic moiety, or a hydroxyl it is understood that R₂ can additionally comprise a salt or a protecting group.

One of ordinary skill in the art of metabolic engineering will appreciate that the figures provided herein describe multiple different pathways that can be used to arrive at the same functionalized alpha substituted acrylates, dicarboxylic acids and intermediates thereof. These pathways can include enzymatic steps that rely upon an endogenous enzyme activity, for example with reference to FIG. 3 step A, if the host cell is an E. coli cell the initial transaminase step can rely upon the endogenous E. coli transaminase (genbank number CAA27279). Similarly, the activity of the endogenous gene can be altered through recombinant techniques to increase or decrease the endogenous transaminase activity in the host cell.

FIGS. 3, 5, 9, and 10 show pathways that produce intermediates and end products, such as functionalized alpha substituted dicarboxylic acids and functionalized alpha substituted 3HP. For example, a conversion from one intermediate to another intermediate, such as an amino acid to an alpha keto acid, alpha keto acid to an alpha substituted malic acid, and alpha substituted malic acid to an alpha substituted maleic acid, alpha substituted maleic acid to a beta-substituted malic acid, a beta substituted malic acid to a beta substituted oxaloacetate acid, a beta substituted oxaloacetate acid to a substituted malonic semialdhyde, a substituted malonic semialdhyde to an alpha substituted 3HP, alpha substituted maleic acid to an alpha substituted fumaric acid, and beta substituted malic acid to an alpha keto acid. These conversions can be facilitated chemically or biologically.

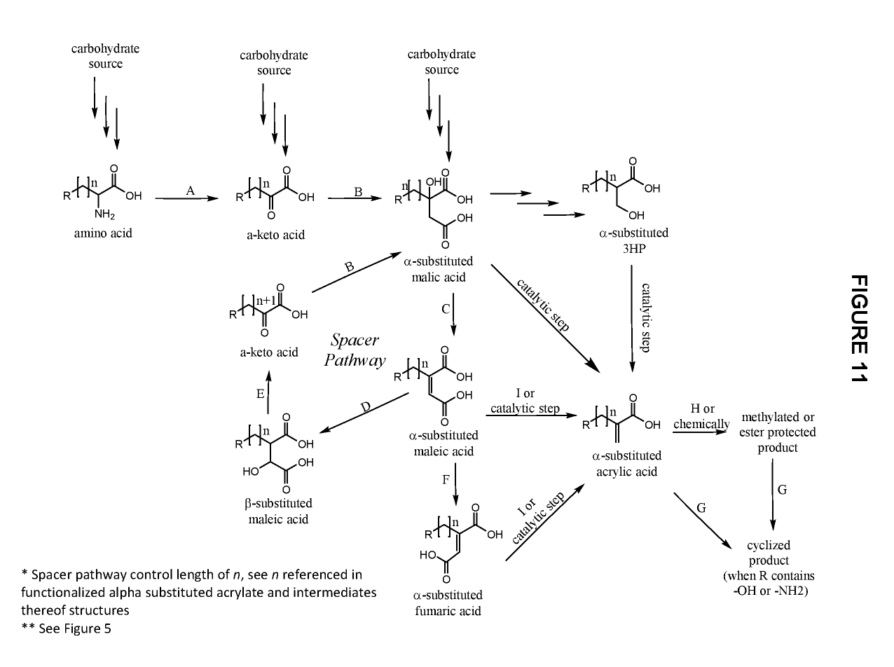

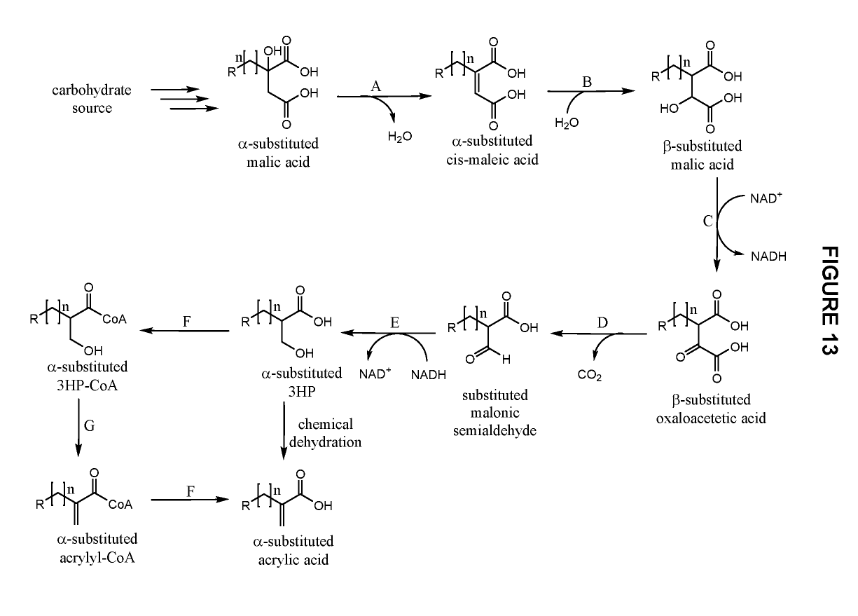

FIGS. 11, 13, 15, 17 and 19 show pathways that produce intermediates and end products, such as described above with reference to FIGS. 3, 5, 9, and 10. FIGS. 11, 13, 15, 17 and 19, however, also show fully biological metabolic pathways that are useful for making functionalized alpha substituted acrylic acids as well as salts, esters and lactones thereof. The conversions identified in the associated Figures can be facilitated chemically or biologically.

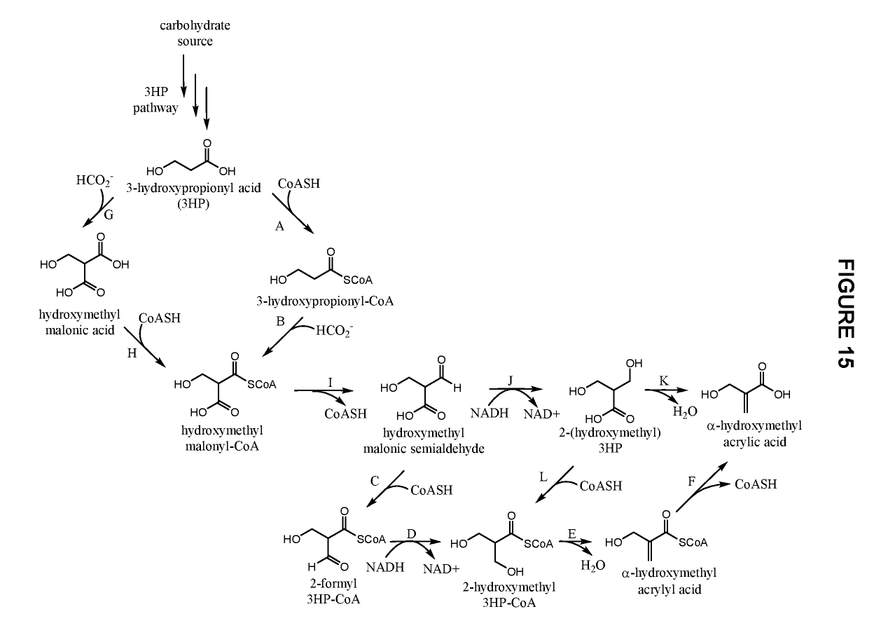

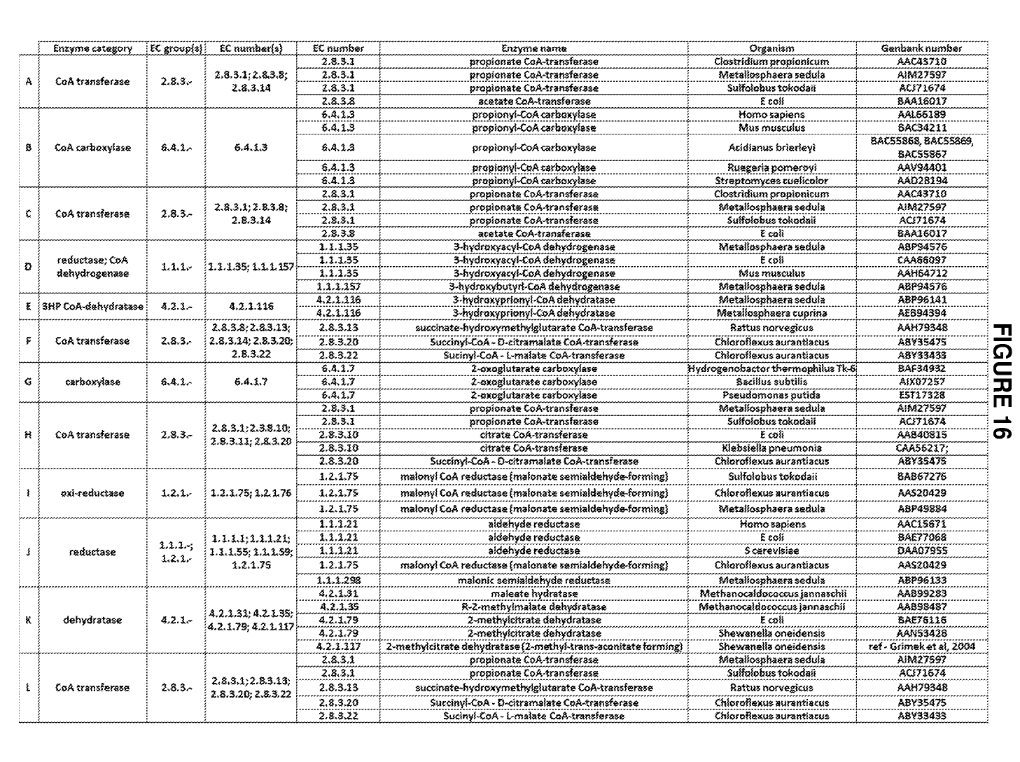

For instance, FIG. 15 shows a fully or partially biological route to making alpha hydroxymethyl acrylic acid. This pathway can be engineered into any host that either has been engineered to, or naturally makes, 3-hydroxypropionic acid. The 3HP can be converted to hydroxymethyl malonic acid and/or 3-hydroxypropionyl-CoA using polypeptides having the enzymatic activities described in FIG. 16, rows G and A, respectively. The hydroxymethyl malonic acid and/or the 3-hydroxypropionyl-CoA can be converted to hydroxymethyl malonyl-CoA using polypeptides having the enzymatic activities described in FIG. 16, rows H and B, respectively. The hydroxymethyl malonyl-CoA can be converted to hydroxymethyl malonic semialdehyde using polypeptides having the enzymatic activities described in FIG. 16, row I.

The hydroxymethyl malonic semialdehyde can be converted to 2-formyl 3HP-CoA using a polypeptide having the activity described in FIG. 16, row C. The 2-formyl 3HP-CoA can be converted to 2-hydroxymethyl 3HP-CoA using a polypeptide having the activity described in FIG. 16, row D. The 2-hydroxymethyl 3HP-CoA can be converted to alpha hydroxymethyl acrylyl acid using a polypeptide having the activity described in FIG. 16, row E. The alpha hydroxymethyl acrylyl acid can be converted to alpha hydroxymethyl acrylic acid using a polypeptide having the activity described in FIG. 16, row F.

The hydroxymethyl malonic semialdehyde can be converted to 2-(hydroxymethyl) 3HP using a polypeptide having the activity described in FIG. 16, row J. The 2-(hydroxymethyl) 3HP can be converted to alpha hydroxymethyl acrylic and/or 2-hydroxymethyl 3HP-CoA using apolypeptide(s) using polypeptides having the activity described in FIG. 16, rows K and L, respectively. The 2-hydroxymethyl 3HP-CoA can follow the pathway described above to alpha substituted acrylic acid. The conversion of 2-(hydroxymethyl)-3HP to alpha-hydroxymethyl acrylic acid can be effected by using an enzyme having the activity described in FIG. 16, row K.

In one alternative, 2-(hydroxymethyl)-3HP is isolated and converted to alpha-hydroxymethyl acrylic acid via chemical dehydration, e.g. where step K in FIG. 15 is a chemical dehydration step.

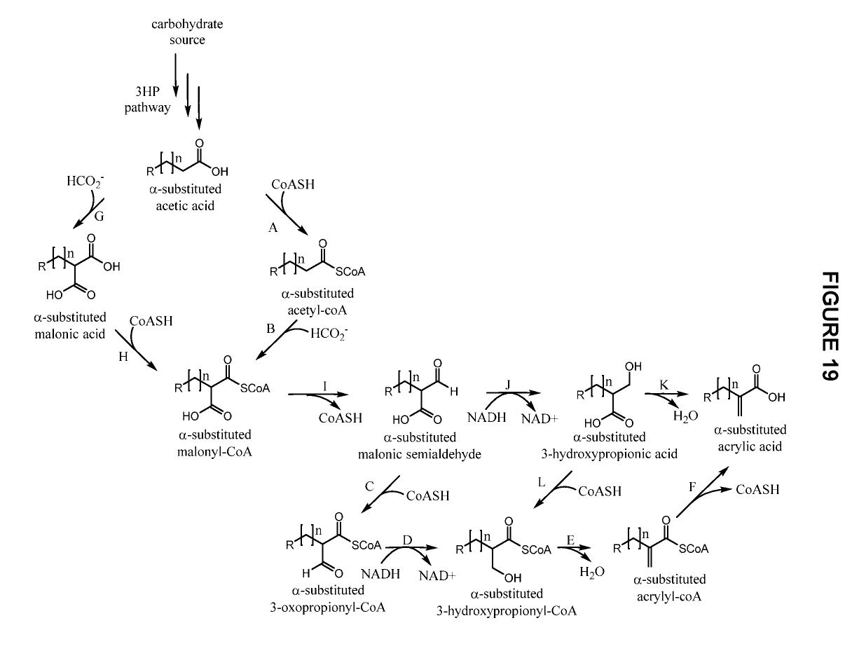

Similarly, FIG. 19 shows a fully or partially biological route to making alpha-substituted acrylic acids or an intermediate thereof. For instance, steps A to L are as shown in FIG. 16. In one alternative, the intermediate alpha-substituted 3-hydroxypropionic acid is isolated and converted to alpha-substituted acrylic acid via chemical dehydration, e.g. where step K in FIG. 19 refers to a chemical dehydration step.

One of ordinary skill in the art will appreciate that the enzymes (as used herein enzymes are interchangeably referred to as polypeptides having activity) identified in the figures and elsewhere herein are exemplary enzymes and that their activities and substrate specificity can be easily tested and altered. Moreover, new enzymes having the same activities will be identified in the future and that such future discovered enzymes can be used in the described pathways.

In some examples, polypeptides having one or more point mutations that allow the substrate specificity and/or activity of the polypeptides to be modified, are used to make intermediates and products.

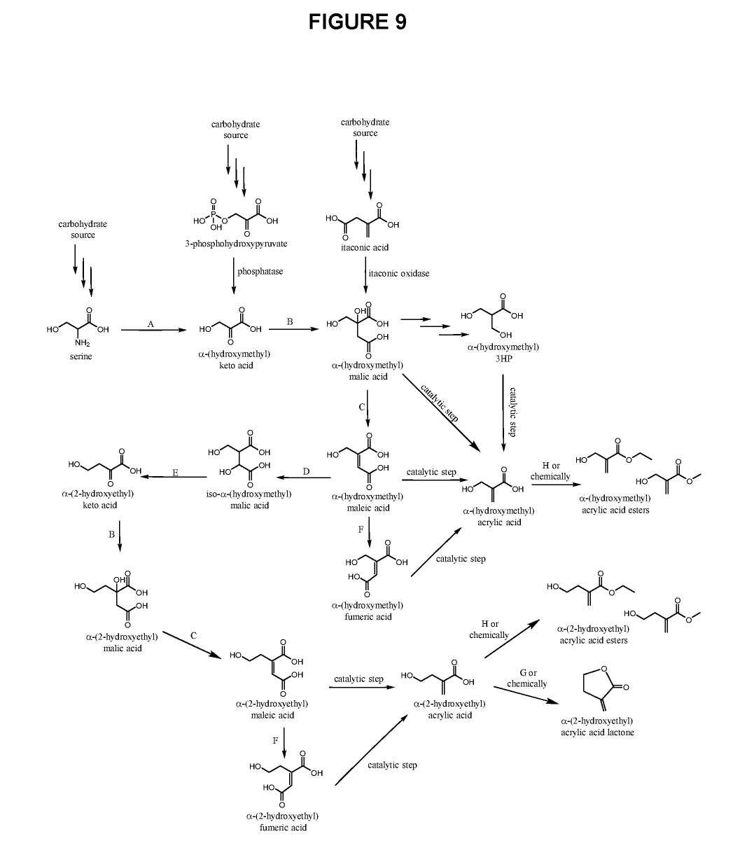

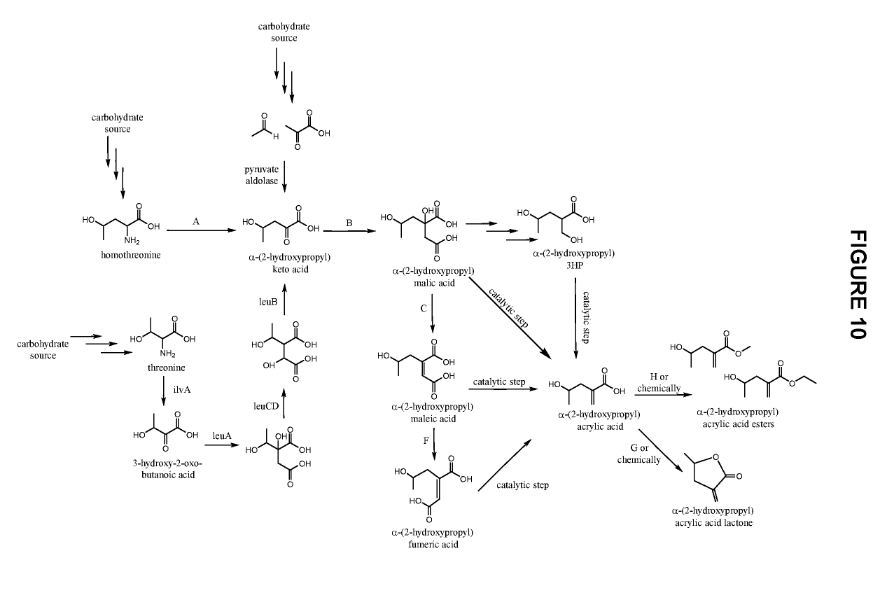

For clarity relating to FIG. 3, there are multiple pathways shown that can give rise to one or more functionalized alpha substituted dicarboxylic acids and alpha substituted 3HP molecules. These pathways vary depending upon the particular desired product. For clarity FIGS. 9 and 10 provide structures and enzymes that illustrate the use of the pathways for creating specific products.

FIG. 9 shows a pathway that can be used to make alpha (hydroxymethyl) dicarboxylic acids starting from serine, 3-phosphohydroxypyruvate or itaconic acid. FIG. 9 also provides an example of how the spacer pathway (see FIG. 3) can be used to add a carbon to the alpha functional group from alpha (hydroxymethyl) maleic acid to produce alpha (hydroxyethyl) maleic acid and or alpha (hydroxyethyl) fumaric acid.

Similarly, FIG. 10 shows a pathway that can be used to produce alpha (2-hydroxypropyl) dicarboxylic acids and alpha (2-hydroxypropyl) 3HP starting from homothreonine, threonine via additional spacer pathway steps or pyruvate.

One of ordinary skill in the art will appreciate that a mix of functionalized alpha substituted dicarboxylic acids and 3HP can be produced by a recombinant microorganism and that the mix can be chemically converted to the functionalized alpha substituted acrylate. The functionalized alpha substituted acrylate can then be subsequently converted chemically or enzymatically to an ester or a lactone as shown in FIGS. 3, 9 and 10. The enzymes responsible for these conversions are shown in rows H and G of FIG. 4.

A variety of different carbons sources could be used to make the desired product depending upon the strain that is chosen to make the recombinant microorganism described herein. For example, a host strain that naturally can utilize organic acids, sugar alcohols, and/or celluloses can be used so that upon introduction of the desired pathway the product is produced from a particular carbon source. The variety of different carbon sources that can be used is indicated by the multiple stacked arrows indicated in FIG. 3. One of ordinary skill in the art will appreciate that the arrows indicate that a variety of different enzymatic activities will need to be utilized by the recombinant microorganism and that which particular activities are needed will depend upon the source of the carbohydrate used. Additionally, a recombinant microorganism can be engineered to utilize a particular carbon source by engineering into the recombinant microorganism known enzymatic activities. For example, if it is desired to produce a product from xylose the enzymatic activities described in WO2014164410 can be introduced into the recombinant microorganism. Similarly, methane, methanol, glucose, acetic acid, as well as other organic molecules can be used by a recombinant microorganism through introduction of specific transporters and other enzymatic activities.

The biosynthetic pathways described herein can be engineered into host organisms that naturally, or have already been engineered to, overproduce an intermediate in the pathway. For example, a host cell that already produced a high concentration of pyruvate, itaconic acid, or an amino acid can be chosen for use as the recombinant host cell into which one or more recombinant nucleic acid sequences will be included to produce the desired functionalized alpha substituted dicarboxylic acids and functionalized alpha substituted 3HP. For example, the following titers of amino acids are already being obtained through various fermentations.

TABLE B
Top titers of amino acid fermentations*

| Amino Acid | Titer (g/l) |
|---|---|
| L alanine | 114 |
| L arginine | 96 |
| L glutamic acid | 130 |
| L glutamine | 49 |
| L histidine | 42 |
| L hydroxyproline | 41 |
| L isoleucine | 40 |
| L leucine | 34 |
| L lysine HCl | 170 |
| L phenylalanine | 51 |
| L proline | 100 |
| L serine | 65 |
| L threonine | 100 |
| L tryptophan | 60 |
| L tyrosine | 55 |
| L valine | 105 |

*Table B is reproduced from Table 2 in Adrio and Demain, Bioengineered Bugs 116-131, March April 2010, which is herein incorporated by reference.

One of ordinary skill in the art will appreciate that regardless of the carbon source(s) used in the fermentation broth to support growth of the recombinant microorganism the economic reality is that there is a desire to maximize the carbon utilization from that carbon source(s) for product production. Generally, this is done by attenuating or completely disrupting unwanted biosynthetic pathways that are otherwise native in the wild type host strain. The desired pathway will be engineered to divert carbon flow because the engineered pathway will have an increased level of enzymatic activity for a substrate that is normally found in the host cell. For example, the recombinant microorganism can display increased activity for alpha ketoglutarate, or alternatively for an amino acid. One of ordinary skill in the art can then review which pathways cause a diversion of carbon from central metabolism up stream or prior to the branch point for the engineered pathway. These diverting pathways can then be attenuated or knocked out so that more carbon is funneled to the desired product. Examples, of pathways that can be attenuated or knocked out include pathways to products such as ethanol, acetate, glycerol and the like (see examples in WO2008116853). More specific examples of activities that can be attenuated include those associated with the following enzymes: pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (IdhA). The attenuation of these enzymes as well as other methods that can be used to increase the produce of functionalized alpha substituted dicarboxylic acids and functionalized 3-hydroxypropionic acid are described in WO2013163292. WO2013163292 to Man Kit Lau also describes the use of the spacer pathway shown in FIG. 3 to elongate the keto acid, alpha keto adipate to alpha keto pimalate. This same pathway can be used to elongate the functionalized alpha substituted dicarboxylic acids (WO2013163292 is herein incorporated by reference).

The design of a commercially viable biosynthetic pathway should have sufficient yield of product compared to the consumed carbon source and it should also be capable of producing the product in a balanced manner. Meaning that the overall products and cofactors consumed and produced by the recombinant microorganism should result in no net surplus or deficit which would tax to host cells ability to produce the product. For example if the overall pathway consumes acetyl CoA, an additional source of acetyl CoA may need to be engineered into the pathway. Alternatively, if an excess of a particular co-product, for example H₂O₂ results, an appropriate mechanism for transporting the co-product or consuming the co-product should be included in the pathway.

II. Chemical Catalysis

The following examples are provided to illustrate the invention, but are not intended to limit the scope thereof. All parts and percentages are by weight unless otherwise indicated.

The methods provided herein relate to the conversion of functionalized alpha substituted C4 dicarboxylic acid to functionalized acrylic acid and derivatives of functionalized alpha substituted C4 organic acids (see Table A above). For example, the preparation of functionalized acrylic acid can be as shown in Scheme 1.

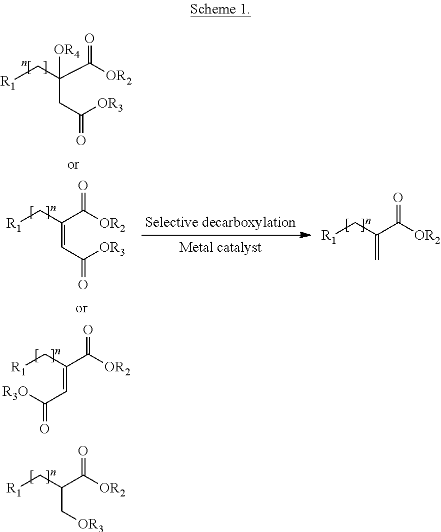

wherein each of the compounds may be present as a salt or ester thereof.

Accordingly, provided herein are methods for making functionalized acrylic acid, or a salt or ester thereof, the method comprising contacting a functionalized alpha substituted C4 acid, or a salt, ester, or lactone thereof, with a metal catalyst.

In some embodiments, a method for making a compound of Formula I:

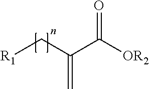

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, and n is equal 1 or greater. The method comprising contacting a metal catalyst with a composition comprising a compound of Formula II, III, IV:

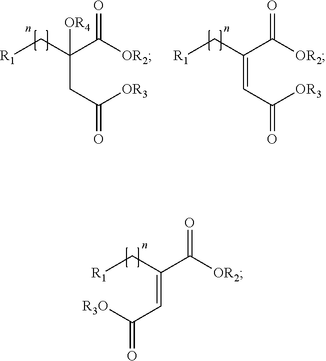

or a salt thereof,

wherein:

each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂, R₃, R₄ is individually selected from H and a protecting group and n is greater than 1.

As shown in Scheme 1, it is thought that a compound of Formula I, or a salt thereof, can be prepared in some embodiments by a method comprising selective decarboxylation of the beta carboxylate of the compound of Formula II, III, or IV to prepare a compound of Formula I, or a salt thereof.

This disclosure further provides a method for making functionalized acrylic acid, or a salt or ester thereof, the method comprising contacting a functionalized alpha substituted C4 dicarboxylic acid with a metal catalyst. In some embodiments, a method for making a compound of Formula I, or a salt thereof, includes contacting a metal catalyst with composition comprising a compound of Formula II, III, IV, or a salt thereof. For example, a method for making a compound of Formula I, or a salt thereof, can include selective decarboxylation of the compound of Formula II, III, IV to make a compound of Formula I, or a salt thereof. In some embodiments, such a method is performed in a single reaction pot in the presence of a metal catalyst.

Also provided herein are methods for making compounds as depicted in Table A above, or a salt or ester thereof. The methods can include contacting a functionalized alpha substituted C4 dicarboxylic acid, or a salt, ester, or lactone thereof, with a metal catalyst. In some embodiments, a method for making a compound selected from:

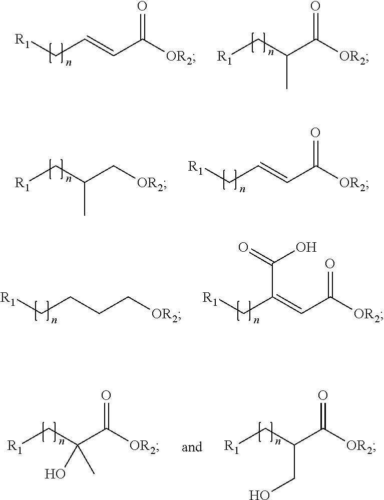

or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, and n is equal to 1 or greater.

In some embodiments, a method for making a compound depicted in Table A above, or a salt thereof, can include selective decarboxylation of the compound of Formula II, III, IV to make a compound of Formula I, or a salt thereof.

In further embodiment, a method of making an alpha-substituted acrylic acid of the formula:

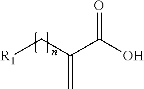

or a salt or ester thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater;
the method comprising dehydrating an alpha-substituted 3-hydroxypropionic acid to produce the alpha-substituted acrylic acid, wherein the alpha-substituted 3-hydroxypropionic acid is of the formula:

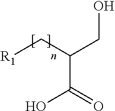

or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater. For instance, the 3-hydroxypropionic acid is produced by a method comprising culturing a recombinant microorganism as herein defined in the presence of a carbohydrate; and separating the alpha-substituted 3-hydroxypropionic acid or ester or salt thereof.

The methods provided herein can be used to prepare one or more of the compounds described herein. For example, the methods described herein can be used to prepare a composition comprising two or more compounds selected from the group consisting of compounds depicted in Table A, or a salt or ester thereof. In some embodiments, the method comprises contacting functionalized alpha substituted C4 dicarboxylic acid, or a salt, ester, or lactone thereof, with a metal catalyst. In some embodiments, a method is provided for making a composition comprising two or more compounds selected from the group consisting of those shown in Table A: or a salt thereof, wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, the method comprising contacting a metal catalyst with a composition comprising a compound of Formula II, III, IV, or a salt thereof. The skilled artisan will be aware that some functional groups could be sensitive to catalysis addition. Introduction of protecting groups could be necessary, such as in the case of R₁.

In some embodiments, a method for making a composition comprising compounds of Formula I and one or more compounds depicted in Table A, or salts thereof, can include selective decarboxylation of the beta carboxylate of the compound of Formula II, III, or IV.

In the compounds described above (i.e., compounds of Formula I, II, III, IV), reference is made to a protecting group. In some embodiments, a carboxyl group may be protected (e.g., in the case of R₁, R₂, and R₃). For this purpose, R₂, R₃, and R₄ may include any suitable carboxyl protecting group including, but not limited to, esters, amides, or hydrazine protecting groups. Each occurrence of the protecting group may be the same or different.

In particular, the ester protecting group may include methyl, methoxy methyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethylbenzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylmethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The amide and hydrazine protecting groups may include N,N-dimethylamide, N-7-nitroindoylamide, hydrazide, N-phenylhydrazide, and N,N′-diisopropylhydrazide.

In some embodiments, a hydroxyl group may be protected (e.g., in the case of R₁ or R₄). For this purpose, R₄ may include any suitable hydroxyl protecting group including, but not limited to, ether, ester, carbonate, or sulfonate protecting groups. Each occurrence of the protecting group may be the same or different.

In particular, the ether protecting group may include methyl, methoxy methyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethylbenzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylemethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The ester protecting group may include acetoxy (OAc), aryl formate, aryl acetate, aryl levulinate, aryl pivaloate, aryl benzoate, and aryl 9-fluoroenecarboxylate. In one embodiment, the ester protecting group is an acetoxy group.

The carbonate protecting group may include aryl methyl carbonate, 1-adamantyl carbonate (Adoc-OAr), t-butyl carbonate (BOC-OAr), 4-methylsulfinylbenzyl carbonate (Msz-OAr), 2,4-dimethylpent-3-yl carbonate (Doc-OAr), aryl 2,2,2-trichloroethyl carbonate, aryl vinyl carbonate, aryl benzyl carbonate, and aryl carbamate.

The sulfonate protecting groups may include aryl methanesulfonate, aryl toluenesulfonate, and aryl 2-formylbenzenesulfonate.

Preparation of compounds as described herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997) (each of which is incorporated herein by reference in their entirety.

In the methods described above, functionalized alpha substituted C4 dicarboxylic acid, or a salt, ester, or lactone thereof, may be obtained by methods known by those of ordinary skill in the art. For example, the functionalized alpha substituted C4 dicarboxylic acid, or a salt, ester, or lactone thereof, may be obtained commercially or may be produced synthetically. In some embodiments, the functionalized alpha substituted C4 dicarboxylic acid, or a salt, ester, or lactone thereof, may be prepared using fermentation methods such as those described in WO 2014/043182 assigned to BioAmber Inc., which is incorporated by reference in its entirety herein.

A metal catalyst as used herein can include any suitable metal catalyst. For example, a suitable metal catalyst would include one that can facilitate the conversion of functionalized alpha substituted C4 dicarboxylic acid, or a salt, ester, or lactone thereof, to one or more of functionalized acrylic acids, salts, esters or lactones thereof.

In some embodiments, a suitable metal catalyst for the present methods is a heterogeneous (or solid) catalyst. The metal catalyst (e.g., a heterogeneous catalyst) can be supported on at least one catalyst support (referred to herein as “supported metal catalyst”). When used, at least one support for a metal catalyst can be any solid substance that is inert under the reaction conditions including, but not limited to, oxides such as silica, alumina and titania, compounds thereof or combinations thereof; barium sulfate; zirconia; carbons (e.g., acid washed carbon); and combinations thereof. Acid washed carbon is a carbon that has been washed with an acid, such as nitric acid, sulfuric acid or acetic acid, to remove impurities. The support can be in the form of powders, granules, pellets, or the like. The supported metal catalyst can be prepared by depositing the metal catalyst on the support by any number of methods well known to those skilled in the art, such as spraying, soaking or physical mixing, followed by drying, calcination, and if necessary, activation through methods such as heating, reduction, and/or oxidation. In some embodiments, activation of the catalyst can be performed in the presence of hydrogen gas. For example, the activation can be performed under hydrogen flow or pressure (e.g., a hydrogen pressure of about 200 psi). In some embodiments, the metal catalyst is activated at a temperature of about 100° C. to about 500° C. (e.g., about 100° C. to about 500° C.).

In some embodiments, the loading of the at least one metal catalyst on the at least one support is from about 0.1 weight percent to about 20 weight percent based on the combined weights of the at least one acid catalyst plus the at least one support. For example, the loading of the at least one metal catalyst on the at least one support can be about 5% by weight.

A metal catalyst can include a metal selected from nickel, palladium, platinum, copper, zinc, rhodium, ruthenium, bismuth, iron, cobalt, osmium, iridium, vanadium, and combinations of two or more thereof. In some embodiments, the metal catalyst comprises copper or platinum. For example, the metal catalyst can comprise platinum.

A chemical promoter can be used to augment the activity of the catalyst. The promoter can be incorporated into the catalyst during any step in the chemical processing of the catalyst constituent. The chemical promoter generally enhances the physical or chemical function of the catalyst agent, but can also be added to retard undesirable side reactions. Suitable promoters include, for example, sulfur (e.g., sulfide) and phosphorous (e.g., phosphate). In some embodiments, the promoter comprises sulfur.

Non-limiting examples of suitable metal catalysts as described herein are provided in Table C.

TABLE C

| A/a | Product | Description | Company | Batch No |
|---|---|---|---|---|
| 1 | RANEY Ni 4.2 | Ni Catalyst | W.R. Grace | NA |
| 2 | Cu-0860 | Cu Catalyst | BASF | NA- |
| | E 1/16″ 3F | (unreduced as oxide) | | |
| 3 | Cu-0865 | Cu Catalyst | BASF | NA- |
| | T 3/16″ | (unreduced as oxide) | | |
| 4 | F51-8PPT | Cu/Zn/Al MeOH | Synetics | NA |
| | | (unreduced as oxides) | Johnson | |
| | | | Matthey | |
| | | | Catalysts | |
| 5 | 10% Pd/C | 10% Pd on Carbon | Johnson | |
| | A402028-10 | (51.47% H₂O) | Matthey | |
| | | | Catalysts | |
| 6 | 5% Pd/C | 5% Pd on Carbon | Johnson | |
| | A401102-5 | (56.34% H₂O) | Matthey | |
| | | | Catalysts | |
| 7 | 5% Pd/C | 5% Pd on Carbon | Johnson | |
| | A405828-5 | (47.22% H₂O) | Matthey | |
| | | | Catalysts | |
| 8 | 5% Pd/C | 5% Pd on Carbon | Johnson | |
| | A405032-5 | (67.86% H₂O) | Matthey | |
| | | | Catalysts | |
| 9 | 5% Pd/C | 5% Pd on Carbon | Johnson | |
| | A405038-5 | (64.81% H₂O) | Matthey | |
| | | | Catalysts | |
| 10 | 5% Pd/C | 5% Pd on Carbon | Johnson | |
| | A503023-5 | (54.36% H₂O) | Matthey | |
| | | | Catalysts | |
| 11 | 5% Pd/C | 5% Pd on Carbon | Johnson | |
| | A503032-5 | (65.72% H₂O) | Matthey | |
| | | | Catalysts | |
| 12 | 5% Pd/C | 5% Pd on Carbon | Johnson | |
| | A503038-5 | (63.41% H₂O) | Matthey | |
| | | | Catalysts | |
| 13 | 5% Pd/C | 5% Pd on Carbon | Johnson | |
| | A102023-5 | (55.98% H₂O) | Matthey | |
| | | | Catalysts | |
| 14 | 5% Pd/C | 5% Pd on Carbon | Johnson | |
| | A102038-5 | (64.57% H₂O) | Matthey | |
| | | | Catalysts | |
| 15 | 5% Pd (S)/C | 5% Pd on Carbon, | Johnson | |
| | A103038-5 | Sulfided | Matthey | |
| | | (59.74% H₂O) | Catalysts | |
| 16 | 5% Pd/Al₂O₃ | 5% Pd on alumina | Johnson | |
| | A302011-5 | (0.46% H₂O) | Matthey | |
| | | | Catalysts | |
| 17 | 5% Pd/Al₂O₃ | 5% Pd on alumina | Johnson | |
| | A302099-5 | (0.52% H₂O) | Matthey | |
| | | | Catalysts | |
| 18 | 5% Pd/CaCO₃ | 5% Pd on calcium | Johnson | |
| | A302060-5 | carbonate | Matthey | |
| | | (0.73% H₂O) | Catalysts | |
| 19 | 5% | 5% Pd on calcium | Johnson | |
| | Pd(Pb)/CaCO₃ | carbonate with lead | Matthey | |
| | A305060-5 | (0.69% H₂O) | Catalysts | |
| 20 | 5% | 5% Pd on calcium | Johnson | |
| | Pd(Pb)/CaCO₃ | carbonate with lead | Matthey | |
| | A306060-5 | (0.72% H₂O) | Catalysts | |
| 21 | 5% Pd/BaSO₄ | 5% Pd on barium | Johnson | |
| | A308053-5 | sulfate | Matthey | |
| | | (0.75% H₂O) | Catalysts | |
| 22 | 4% Pd-1% Pt/C | 4% Pd & 1% Pt | Johnson | |
| | E101049-4/1 | on carbon | Matthey | |
| | | (54.30% H₂O) | Catalysts | |
| 23 | 4% Pd-1% Pt/C | 4% Pd & 1% Pt | Johnson | |
| | E101023-4/1 | on carbon | Matthey | |
| | | (55.88% H₂O) | Catalysts | |
| 24 | 4.5% Pd-0.5% | 4.5% Pd & 0.5% Rh | Johnson | |
| | Rh/C | on carbon | Matthey | |
| | F101032-4.5/0.5 | (61.48% H₂O) | Catalysts | |
| 25 | 4.5% Pd- | 4.5% Pd and 0.5% Rh | Johnson | |
| | 0.5% Rh/C | on Carbon | Matthey | |
| | F101038-4.5/0.5 | (52.51% H₂O) | Catalysts | |
| 26 | 3% Pt/C | 3% platinum on | Johnson | |
| | B103032-3 | carbon | Matthey | |
| | | (67.93% H₂O) | Catalysts | |
| 27 | 5% Pt/C | 5% platinum on | Johnson | |
| | B103032-5 | carbon | Matthey | |
| | | (59.45% H₂O) | Catalysts | |
| 28 | 5% Pt/C | 5% platinum on | Johnson | |
| | B103018-5 | carbon | Matthey | |
| | | (55.90% H₂O) | Catalysts | |
| 29 | 5% Pt/C | 5% platinum on | Johnson | |
| | B102022-5 | carbon | Matthey | |
| | | (46.67% H₂O) | Catalysts | |
| 30 | 5% Pt/C | 5% platinum on | Johnson | |
| | B104032-5 | carbon | Matthey | |
| | | (62.06% H₂O) | Catalysts | |
| 31 | 5% Pt/C | 5% platinum on | Johnson | |
| | B501032-5 | carbon | Matthey | |
| | | (67.49% H2O) | Catalysts | |
| 32 | 5% Pt/C | 5% platinum on | Johnson | |
| | B501018-5 | carbon | Matthey | |
| | | (54.01% H₂O) | Catalysts | |
| 33 | 5% Pt(Bi)/C | 5% platinum& Bismuth | Johnson | |
| | B503032-5 | 5% on carbon | Matthey | |
| | | (59.40% H₂O) | Catalysts | |
| 34 | 5% Pt(S)/C | 5% platinum on carbon, | Johnson | |
| | B109032-5 | Sulfide wet | Matthey | |
| | | (60.68% H₂O) | Catalysts | |
| 35 | 5% Pt(S)/C | 5% platinum on carbon, | Johnson | |
| | B106032-5 | Sulfide wet | Matthey | |
| | | (62.09% H₂O) | Catalysts | |
| 36 | 5% Pt/Al2O3 | 5% platinum on | Johnson | |
| | B301013-5 | alumina | Matthey | |
| | | (2.84% H₂O) | Catalysts | |
| 37 | 5% Pt/Al₂O₃ | 5% platinum on | Johnson | |
| | B301099-5 | alumina | Matthey | |
| | | (3.28% H₂O) | Catalysts | |
| 38 | 5% Rh/C | 5% rhodium on | Johnson | |
| | C101023-5 | carbon | Matthey | |
| | | (47.61% H₂O) | Catalysts | |
| 39 | 3% Rh/C | 5% rhodium on | Johnson | |
| | C101038-5 | carbon | Matthey | |
| | | (64.21% H₂O) | Catalysts | |
| 40 | 5% Rh/Al₂O₃ | 5% rhodium on | Johnson | |
| | C301011-5 | alumina | Matthey | |
| | | (4.74% H₂O) | Catalysts | |
| 41 | 5% Ru/C | 5% ruthenium on carbon | Johnson | |
| | D101023-5 | (62.59% H₂O) | Matthey | |
| | | | Catalysts | |
| 42 | 5% Ru/C | 5% ruthenium on | Johnson | |
| | C101002-5 | carbon | Matthey | |
| | | (58.46% H₂O) | Catalysts | |
| 43 | 5% Ru/Al₂O₃ | 5% ruthenium on | Johnson | |
| | D302011-5 | alumina | Matthey | |
| | | (0.99% H₂O) | Catalysts | |
| 44 | 5% Ra- | 5% ruthenium & 0.25 | Johnson | |
| | 0.25% Pd/C | palladium on carbon | Matthey | |
| | C102038-5 | (53.15% H₂O) | Catalysts | |
| 45 | F105N/W 5% | 5% Pt on activated Carbon | Evonik | |
| | | (55% H₂O) | | |
| 46 | F1082 QHA/W | 3% Pt on activated Carbon | Evonik | |
| | 3% | (63.5% H₂O) | | |
| 47 | F1015 RE/W 5% | 5% Pt on activated Carbon | Evonik | |
| | | (62.3% H₂O) | | |
| 48 | CF 1082 BV/W | 1% Pt + 2% Vanadium on | Evonik | |
| | 1% Pt + 2% V | activated carbon | | |
| | | (61.5% H₂O) | | |
| 49 | G106 N/W 5% | 5% Rh on activated Carbon | Evonik | |
| | | (65.4% H₂O) | | |
| 50 | H198 P/W | 5% Ru on activated Carbon | Evonik | |
| | 5% Ru % | (58.7% H₂O) | | |
| 51 | Noblyst P1093 | 5% Palladium on activated | Evonik | |
| | 5% | Carbon (55.5% H₂O) | | |
| 52 | Noblyst P1070 | 10% Palladium on activated | Evonik | |
| | 5% | Carbon (53.5% H₂O) | | |
| 53 | Noblyst P1092 | 5% Palladium activated | Evonik | |
| | 5% | Carbon | | |
| | | (55.5% H₂O) | | |
| 54 | Noblyst P1109 | 5% Palladium on activated | Evonik | |
| | 5% | Carbon (55.6% H₂O) | | |
| 55 | Noblyst P1090 | 5% Palladium on activated | Evonik | |
| | 5% | Carbon (53.5% H₂O) | | |
| 56 | Noblyst P1086 | 5% Palladium on activated | Evonik | |
| | 5% | Carbon (55% H₂O) | | |
| 57 | 46-1710 CAS# | 0.6% Palladium on activated | | |
| | 7440-05-3 | Carbon, unreduced(50% H₂O | | |
| | | wet paste) | | |
| 58 | 46-1901 CAS# | 5% Palladium on activated peat | | |
| | 7440-05-3 | Carbon, unreduced(50% H₂O | | |
| | | wet paste) | | |
| 59 | 46-1902 CAS# | 5% Palladium on activated | | |
| | 7440-05-3 | wood Carbon, reduced, dry | | |
| 60 | 46-1903 CAS# | 5% Palladium on activated | | |
| | 7440-05-3 | wood Carbon, reduced, 50% | | |
| | | water wet paste | | |
| 61 | 46-1904 CAS# | 5% Palladium on activated | | |
| | 7440-05-3 | wood Carbon, unreduced(50% | | |
| | | H₂O wet paste) | | |
| 62 | 46-1905 CAS# | 10% Palladium on activated | | |
| | 7440-05-3 | wood Carbon, reduced(50% | | |
| | | H₂O wet paste) | | |
| 63 | 46-1951 CAS# | 5% Palladium on alumina | | |
| | 7440-05-3 | Al₂O₃, reduced | | |
| 64 | 46-1707 CAS# | 20% Palladium on activated | | |
| | 7440-05-3 | carbon Pearlman's catalyst, | | |
| | | unreduced, 50% water wet | | |
| | | paste | | |
| 65 | 78-1611 CAS# | 5% Platinum on activated wood | | |
| | 7440-06-4 | carbon, reduced, dry | | |
| 66 | 78-1612 CAS# | 5% Platinum on activated wood | | |
| | 7440-06-4 | carbon, reduced, 50% H₂O | | |
| | | wet paste | | |
| 67 | 78-1613 CAS# | 5% Platinum on activated | | |
| | 7440-06-4 | carbon, unreduced, 50% H₂O | | |
| | | wet paste | | |
| 67 | 44-4065 CAS# | 5% Ruthenium on activated | | |
| | 7440-18-8 | carbon, reduced, 50% H₂O | | |
| | | wet paste | | |
| 68 | 45-1875 CAS# | 5% Rhodium on activated wood | | |
| | 7440-16-6 | carbon, reduced, 50% H₂O wet | | |
| | | paste | | |

Temperature, solvent, catalyst, reactor configuration, pressure and mixing rate are all parameters that can affect the conversions described herein. The relationships among these parameters may be adjusted to effect the desired conversion, reaction rate, and selectivity in the reaction of the process.

In some embodiments, the methods provided herein are performed at temperatures from about 25° C. to about 350° C. For example, the methods can be performed at a temperature of at least about 100° C. In some embodiments, a method provided herein is performed at a temperature of about 100° C. to about 200° C. For example, a method can be performed at a temperature of about 150° C. to about 180° C.

The methods described herein may be performed neat, in water or in the presence of an organic solvent. In some embodiments, the reaction solvent comprises water. Exemplary organic solvents include hydrocarbons, ethers, and alcohols. In some embodiments, alcohols can be used, for example, lower alkanols, such as methanol and ethanol. The reaction solvent can also be a mixture of two or more solvents. For example, the solvent can be a mixture of water and an alcohol.

The methods provided herein can be performed under inert atmosphere (e.g., N₂ and Ar). In some embodiments, the methods provided herein are performed under nitrogen. For example, the methods can be performed under a nitrogen pressure of about 20 psi to about 1000 psi. In some embodiments, a method as described herein is performed under a nitrogen pressure of about 200 psi.

In some embodiments, additional reactants can be added to the methods described herein. For example, a base such as NaOH can be added to the reaction.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS) or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6) (2004), which is incorporated herein by reference in its entirety) and normal phase silica chromatography.

### EXAMPLES

### Example 1—Cells Resistant to Intermediates

Potential hosts for the described pathways to alpha substituted C4 dicarboxylic acids were identified by determining the tolerance to functionalized alpha substituted dicarboxylic acids as shown in FIG. 2. Specific functionalized alpha substituted C4 dicarboxylic acid compounds were selected to assess tolerance of bacterial and eukaryotic hosts.

Two eukaryotic strains, I. orientalis and S. cerevisiae, were tested at pH 3 and pH 5. Three bacterial strains were tested using their individual optimal conditions. E. coli and C. glutamicum were tested at pH 8 and 30° C., and B. firmus was tested at pH 9 and 37° C. I. orientalis was grown in defined yeast media consisting of 5 g/L ammonium sulfate, 0.5 g/L magnesium sulfate heptahydrate, 3 g/L potassium phosphate monobasic, 10 g/L dextrose, 1 mL/L of 10% glycerol stock, 1 mL/L of 1000× trace. The 1000× trace stock solution contains 4 g/L ZnSO₄.7H₂O, 2 g/L FeSO₄.7H₂O, 1 g/L MnSO₄.H₂O, 0.2 g/L CuSO₄.5H₂O, and 0.8 mL/L H₂SO₄. S. cerevisiae was grown in buffered defined dextrose media consisting of 50 g/L dextrose, 5 g/L yeast extract, and 40 mL/L 25× DM salts. The 25× DM salt stock solution contains 125 g/L ammonium sulfate, 12.5 g/L magnesium sulfate heptahydrate, 75 g/L potassium phosphate monobasic, and 787.5 g/L water. The bacterial strains were grown in standard LB media consisting of 10 g/L bacto-tryptone, 5 g/L yeast extract, 10 g/L NaCl, and 20 g/L dextrose with addition of 20 g/L glucose.

Time points were taken over a period of at least 8 hours and up to 24 hours to calculate the rate of growth. Specific growth rate was determined by plotting the natural logarithm of cell number against time. Tolerance was determined by growth rate of cells in the presence of the compound as compared to in the absence of the compound. The scoring method is indicated in Table D. The results of the tolerance studies are shown in Tables E and F. The second column from the left denotes the maximum concentration of the indicated compound in which growth was detected. The results suggest that S. cerevisiae is a suitable host to produce any of the functionalized alpha substituted C4 diacid compounds tested in this study. I. orientalis is also a suitable host for most of the compounds. The results suggest that E coli is a suitable host to produce homocitrate lactone, and C glutamicum is a suitable host to produce homocitrate, under the conditions tested.

TABLE D
Subjective system used to score effect of
specific chemical on cell growth rate.

| Percent growth | |
|---|---|
| rate as compared | |
| to unchallenged | |
| cells | Score |
| 100-90% | +++ |
| 89-50% | ++ |
| 49-11% | + |
| 10-0% | − |

TABLE E
Tolerance of potential yeast hosts.

| | Max conc | I. orientalis | | S. cerevisiae | |
|---|---|---|---|---|---|
| Compound | (g/L) | pH 3 | pH 5 | pH 3 | pH 5 |
| Homocitrate | 100 | − | − | − | ++ |
| Homocitrate | 100 | ++ | ++ | + | ++ |
| lactone | | | | | |
| 2-isopropylmalic | 50 | ++ | +++ | ++ | +++ |
| acid | | | | | |
| 3-isopropylmalic | 50 | +++ | +++ | +++ | +++ |
| acid | | | | | |

TABLE F
Tolerance of potential bacterial hosts.

| | Max conc | Highest conc with | | | |
|---|---|---|---|---|---|
| | tested | growth observed | | C. | B. |
| Compound | (g/L) | (g/L) | E. coli | glutamicum | firmus |
| Homocitrate | 100 | 50 | − | +++ | − |
| Homocitrate | 100 | 100 | +++ | − | − |
| lactone | | | | | |

### Example 2—Construction of Recombinant Microorganism for Production of Alpha (Hydroxymethyl) Malic Acid Utilizing a Serine Overproducing Microorganism

The microorganism used for production of alpha (hydroxymethyl) malic acid can be selected from fungi, including yeast and filamentous fungi as well as bacteria. The microorganism described in Pharkya et al. can be used as a starting serine overproducing strain for subsequence genetic engineering steps in instances were bacterial production is desired. Similarly, the microorganism described in Stolz et al. and U.S. Ser. No. 00/603,7154A can be used as a starting strain for subsequent genetic engineering steps in instances were eukaryotic production is desired.
Pharkya, Burgard, and Maranas. Exploring the overproduction of amino acids using the bilevel optimization framework optknock. Wiley Intersciences. 24 Nov. 2003.
Stolz et al. Reduced folate supply as key to enhanced L-serine production by Corynebacterium glutamicum. Applied and Environ. Microbio. February 2007, p. 750-755.

The DNA fragments encoding transaminase (FIG. 4, row A) and synthase (FIG. 4, row B), are cloned into an expression vector. Gene candidates and their sequences are shown in FIG. 4, far right column. The transaminase and synthase activity is increased in the recombinant microorganism by introduction of recombinant nucleic acid sequences encoding the identified nucleic acid sequences under the control of appropriate promoters and terminators. Specifically, the transaminase gene is serine-glyoxylate transaminase from Arabidopsis thaliana (EC 2.6.1.45) and the synthase is homocitrate synthase from Schizosaccharomyces pombe (2.3.3.14). The resulting plasmid that successfully transcribes all pathway genes is transformed into a serine overproducing microorganism.

Additionally, expression of a DNA fragment encoding an alpha (hydroxymethyl) malic acid transporter improves production of alpha (hydroxymethyl) malic acid. Specifically, the transporter gene is selected from malic acid transport genes, tehA from E coli (UNIPROT E0IVN4), mae1 from S. pombe (Saayman et al, 2000), and ykxJ from Bacillus subtilis (Krom et al, 2001), or homologs thereof.
Krom, Aardema, and Lolkema. Bacillus subtilis YxkJ is a secondary transporter of the 2-hydroxycarboxylate transporter family that transports L-malate and citrate. J Bacteriol, 2001 October; 183(20):5862-9.
Saayman, van Vuuren, van Zyl, and Viljoen-Bloom. Differential uptake of fumarate by Candida utilis and Schizosaccharaomyces pombe. Appl Microbiol Biotechnol, 2000. 54: 792-798.

### Transaminase Activity Assay

One of ordinary skill in the art will appreciate that the activity of many transaminase enzymes has been characterized and that any method known in the art for detecting transaminase activity can be used. Specifically, upon expression of the Arabidopsis thaliana transaminase that activity can be characterized using the assay described by Kendziorek and Paszkowski. The amount of reaction using glycine as the amino group donor is estimated by determining the remaining 2-oxoacid substrate after the reaction was stopped, which is determined by a spectrophotometric method using NADH and lactate dehydrogenase.
Kendziorek and Paszkowski. Properties of serine:glyoxylate aminotransferase purified from Arabidopsis thaliana leaves. Acta Biochim Biophys Sin, 2008, 40 (2): 102-110.

### Synthase Activity Assay

E coli optimized genes encoding synthases were synthesized and cloned into pTrcHisA (Life Technologies (formerly Invitrogen)). Synthase genes tested are found in Table G. Plasmids containing the optimized synthase genes were transformed into BL21 E. coli cells. Empty plasmid pTrcHisA was also transformed as a negative control. For expression and characterization experiments, shake flasks containing 40 mL TB were inoculated at 5% from overnight cultures. Flasks were incubated at 30° C. at 250 rpm shaking for 2 hours, then protein production was induced with 0.2 mM IPTG and incubated for 4 more hours at 30° C. while shaking. Cells were harvested by centrifugation and pellets were stored at −80° C.

Activity of synthase candidates was assessed with an in vitro assay using DTNB (5,5′-Dithiobis(2-nitrobenzoic acid)) as an indicator. The enzyme activity was tested using either no substrate or hydroxypyruvate as the substrate. The DTNB interacts with the free thio created by the condensation of acetyl-CoA and the substrate present. Unless otherwise specified, all chemicals were purchased from Sigma-Aldrich Chemical Company, St. Louis, Mo.

Cells were lysed using mechanical disruption using a BeadBeater (BopSpec products, Bartlesville, Okla.) following the manufacturer's instructions. The cell lysate was partially clarified by centrifugation (14,000G for 5 minutes). Protein concentrations of the resulting clarified lysates were measured via BioRad total Protein assay using the manufacturer's instructions. Lysates were normalized by protein concentration in 100 mM Tris buffer. The normalized lysates were diluted 1 to 7 in 100 mM Tris buffer. 20 μl of lysate was added to each well for the 96-well plate assay. Each condition was performed in triplicate.

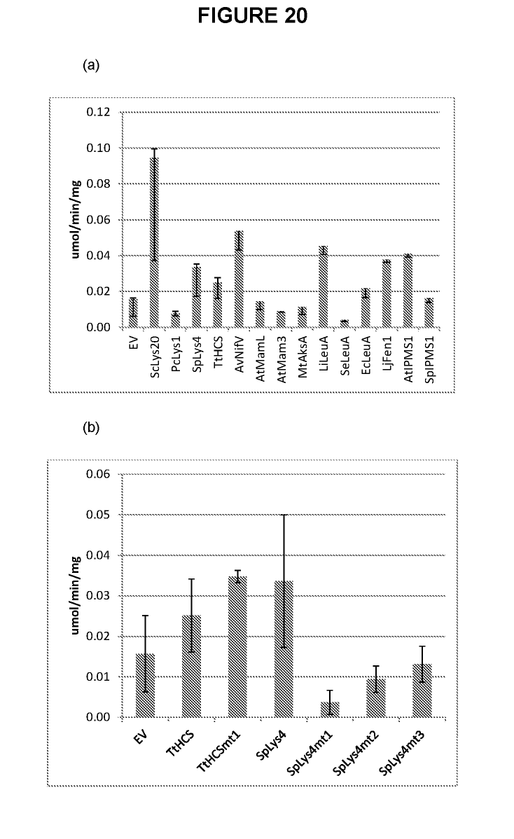

The reaction mixture contains 100 mM Tris pH 7.4, 5 mM MgSO₄, 0.2 mM acetyl-CoA, 0.5 mM DTNB, 0.5 mM substrate, hydroxypyruvate. To start the reaction, 180 μl of reaction mix was added to each well already containing 20 μl lysate. The reactions in these microplates were monitored at 412 nm. Readings were taken every 9 seconds for 10 minutes and the data was used to calculate activities of each enzyme. Results shown in FIG. 20(a). Synthase activity was observed when hydroxypyruvate was the substrate as compared to cells containing empty vector (FIG. 20(a), Table G). Background absorbance as measured by the same reaction with no substrate present were subtracted. Error bars in the graphs reflect the standard deviations calculated for the averages for each condition performed in triplicate. Specific mutations change the activity of the enzymes tested (FIG. 20(b), Table H). Further enzyme engineering will improve specificity and activity of desired enzymatic reaction.

TABLE G
List of candidate synthases and activity with hydroxypyruvate.

| | | | GenBank | Activity with | |
|---|---|---|---|---|---|
| | | | Accession | hydroxy pyruvate | |
| Name | Gene | Organism | Number | (umol/min/mg) | stdev |
| EV | empty vector, | | | 0.016 | 0.009 |
| | ptrc | | | | |
| ScLys20 | lys20 | Saccharomyces cerevisiae | CAA58264 | 0.095 | 0.057 |
| PcLys1 | lys1 | Penicillium chrysogenum | CAP98607 | 0.008 | 0.001 |
| SpLys4 | Lys4 | Schizosaccharomyces pombe | CAB50965 | 0.034 | 0.016 |
| TtHCS | HCS | Thermus thermophilis | AAS81892 | 0.025 | 0.009 |
| AvNifV | NifV | Azotobacter vinelandii | AAA22169 | 0.054 | 0.011 |
| AtMamL | mamL | Arabidopsis thaliana | CAC80102 | 0.015 | 0.005 |
| | (mam1) | | | | |
| AtMam3 | mam3 | Arabidopsis thaliana | AED93108 | 0.009 | 0.001 |
| MtAksA | AksA | Methanothermobacter | AAB86103 | 0.011 | 0.004 |
| | | thermautotrophicus | | | |
| LiLeuA | LeuA/CimA | Leptospira interogans | AAN49401 | 0.045 | 0.005 |
| SeLeuA | LeuA | Salmonella enterica | X51583 | 0.004 | 0.001 |
| EcLeuA | LeuA | Escherichia coli | AAC73185 | 0.022 | 0.005 |
| LjFen1 | FEN1 | Lotus japonicus | BAI49592 | 0.038 | 0.001 |
| AtIPMS1 | IPMS1 | Arabidopsis thaliana | AEE29723 | 0.041 | 0.002 |
| SpIPMS1 | IPMS1 | Schizosaccharomyces pombe | CAW33849 | 0.016 | 0.002 |

TABLE H
List of mutant candidate synthases and activity with hydroxy
pyruvate and comparison with wild type synthase

| | | | | Activity with | |
|---|---|---|---|---|---|
| Name | Gene | Organism | Mutation | hydroxy pyruvate | stdev |
| EV | empty vector, ptrc | | | 0.016 | 0.009 |
| TtHCS | HCS | Thermus thermophilis | | 0.025 | 0.009 |
| TtHCSmt1 | HCS mutant - | Thermus thermophilis | H72L | 0.035 | 0.002 |
| | H72L | | | | |
| SpLys4 | Lys4 | Schizosaccharomyces | | 0.034 | 0.016 |
| | | pombe | | | |
| SpLys4mt1 | Lys4 mutant - | Schizosaccharomyces | D123N | 0.004 | 0.003 |
| | D123N | pombe | | | |
| SpLys4mt2 | Lys4 mutant - | Schizosaccharomyces | D123N, | 0.009 | 0.003 |
| | D123N, V125F | pombe | V125F | | |
| SpLys4mt3 | Lys4 mutant - | Schizosaccharomyces | D123N, | 0.013 | 0.004 |
| | D123N, V125F, | pombe | V125F, | | |
| | I194L | | I194L | | |

Transformation of E coli with Plasmid Containing Nucleic Acid Sequence Encoding Pathway Enzymes

Plasmid DNA molecules are introduced into target E. coli cells engineered with the referenced pathway described in Example 2, above, by chemical transformation or electroporation. For chemical transformation, cells are grown to mid-log growth phase, as determined by the optical density at 600 nm (0.5-0.8). The cells are harvested, washed, and finally treated with CaCl₂. To chemically transform these E coli cells, purified plasmid DNA is allowed to mix with the cell suspension in a microcentrifuge tube on ice. A heat shock is applied to the mixture and followed by a 30-60 min recovery incubation in rich culture medium. For electroporation, E coli cells grown to mid-log growth phase are washed with water several times and finally resuspended into 10% glycerol solution. To electroporate DNA into these cells, a mixture of cells and DNA is pipetted into a disposable plastic cuvette containing electrodes. A short electric pulse is then applied to the cells, which forms small holes in the membrane where DNA can enter. The cell suspension is then incubated with rich liquid medium followed by plating on solid agar plates. Detailed protocol can be obtained in Molecular Cloning: A Laboratory Manual, Third Edition, Sambrook and Russell, 2001, Cold Spring Harbor Laboratory Press, 3^{rd} Edition.

E. coli cells of the BL21 strain are transformed with the described plasmid or plasmids. BL21 is a strain of E. coli having the genotype: B F⁻ dcm ompT hsdS(r_{B}⁻m_{B}⁻) gal [malB+]K-12(λS).

All solutions are prepared in distilled, deionized water. LB medium (1 L) contained Bacto tryptone (i.e. enzymatic digest of casein) (10 g), Bacto yeast extract (i.e. water soluble portion of autolyzed yeast cell) (5 g), and NaCl (10 g). LB-glucose medium contained glucose (10 g), MgSO₄ (0.12 g), and thiamine hydrochloride (0.001 g) in 1 L of LB medium. LB-freeze buffer contains K₂HPO₄ (6.3 g), KH₂PO₄ (1.8 g), MgSO₄ (1.0 g), (NH₄)₂SO₄ (0.9 g), sodium citrate dehydrate (0.5 g) and glycerol (44 mL) in 1 L of LB medium. M9 salts (1 L) contains Na₂HPO₄ (6 g), KH₂PO₄ (3 g), NH₄Cl (1 g), and NaCl (0.5 g). M9 minimal medium contains D-glucose (10 g), MgSO₄ (0.12 g), and thiamine hydrochloride (0.001 g) in 1 L of M9 salts. Antibiotics are added where appropriate to the following final concentrations: ampicillin (Ap), 50 μg/mL; chloramphenicol (Cm), 20 μg/mL; kanamycin (Kan), 50 μg/mL; tetracyclin (Tc), 12.5 μg/mL. Stock solutions of antibiotics are prepared in water with the exceptions of chloramphenicol which is prepared in 95% ethanol and tetracycline which is prepared in 50% aqueous ethanol. Aqueous stock solutions of isopropyl-B-D-thiogalactopyranoside (IPTG) are prepared at various concentrations.

The standard fermentation medium (1 L) contains K₂HPO₄ (7.5 g), ammonium iron (III) citrate (0.3 g), citric acid monohydrate (2.1 g), and concentrated H₂SO₄ (1.2 mL). Fermentation medium is adjusted to pH 7.0 by addition of concentrated NH₄OH before autoclaving. The following supplements are added immediately prior to initiation of the fermentation: D-glucose, MgSO₄ (0.24 g), potassium and trace minerals including (NH₄)₆(Mo₇O₂₄).4H₂O (0.0037 g), ZnSO₄.7H₂O (0.0029 g), H₃BO₃ (0.0247 g), CuSO₄.5H₂O (0.0025 g), and MnCl₂.4H₂O (0.0158 g). IPTG stock solution is added as necessary (e.g., when optical density at 600 nm lies between 15-20) to indicated final concentration. Glucose feed solution and MgSO₄ (1 M) solution are autoclaved separately. Glucose feed solution (650 g/L) is prepared by combining 300 g of glucose and 280 mL of H₂O. Solutions of trace minerals and IPTG are sterilized through 0.22-μm membranes. Antifoam (Sigma 204) is added to the fermentation broth as needed.

Seed inoculant is started by introducing a single colony picked from a LB agar plate into 50 mL TB medium (1.2% w/v Bacto Tryptone, 2.4% w/v Bacto Yeast Extract, 0.4% v/v glycerol, 0.017 M KH₂PO₄, 0.072 M K₂HPO₄). Culture is grown overnight at 37° C. with agitation at 250 rpm until they are turbid. All of the culture conditions include suitable selective pressure to ensure that the plasmid containing the biosynthetic pathway genes is maintained and expressed in the host cell. A 2.5 mL aliquot of this culture is subsequently transferred to 50 mL of fresh TB medium. After culturing at 37° C. and 250 rpm for an additional 3 hours, IPTG is added to a final concentration of 0.2 mM. The resulting culture is allowed to grow at 30° C. for 4 hours. Cells are harvested, washed twice with PBS medium, and resuspended in 0.5 original volume of M9 medium supplemented with glucose (2 g/L). The whole cell suspension is then incubated at 30° C. for 48 h. Samples are taken and analyzed by GC/MS and 1H-NMR.

### Example 3—Construction of Recombinant Microorganism for Production of Alpha (Hydroxymethyl) Malic Acid Starting from Hydroxypyruvate

In addition to the DNA fragments listed in Example 2, the DNA fragment encoding a phosphatase is included. The phosphatase gene is phosphohydroxypyruvate phosphatase selected from yeaB gene from E coli or GPP2 from S cerevisiae (US2011294178A1, WO2010076324A1). The resulting plasmid successfully transcribes all pathway genes for production of alpha (hydroxymethyl) malic acid starting from hydroxypyruvate.

The microorganism used to for production of alpha (hydroxymethyl) malic acid can be selected from fungi, including yeast and filamentous fungi as well as bacteria. To construct a hydroxypyruvate overproducing microorganism, the serC (Uniprot P23721) gene which codes for phosphoserine aminotransferase is deleted. The serC deletion will result in overproduction of 3-phospho hydroxypyruvate, which will be converted by yeaB or GPP2 to hydroxypyruvate. This genetic strategy is used to construct a starting strain for subsequent genetic engineering steps in instances where either bacterial or eukaryotic production is desired. The hydroxypyruvate overproducing organism described here may be used as an alternative to the serine overproducing organism described in Example 2.

Phosphatase activity can be detected using any method known in the art. For example, the assay described in Ho et al. can be used to determine phosphatase activity. Additionally, there are several commercially available kits that are commonly used to measure phosphatase activity.

Ho, Noji, and Saito. Plastidic pathway of serine biosynthesis. Molecular cloning and expression of 3-phosphoserine phosphatase from Arabidopsis thaliana. J Biol chem. 1999 Apr. 16; 274(16):11007-12.

### Example 4—Construction of a Recombinant Microorganism for Production of Itatartaric Acid and/or Hydroxyparaconic Acid

Strains that overproduce alpha alpha (hydroxymethyl) malic acid, also called itatartaric acid, are cultured. Strains that overproduce itaconic and itatartaric and culture conditions are described in Jakubowska et al, 1974; Guevarra and Tabuchi, 1990 a and b; and Geiser et al, 2014. In another iteration, the DNA fragment encoding an itaconic oxidase is overproduced. The itaconic oxidase gene is from Aspergillus or Ustilago (Jakubowska et al, 1974; Guevarra and Tabuchi, 1990 a and b; Geiser et al, 2014). The resulting plasmid successfully transcribes all pathway genes for production of alpha (hydroxymethyl) malic acid, also referred to as itatartaric acid. Mutant forms of the itaconic oxidase gene display increased activity (Aprai, 1958; Aprai, 1959; Jakubowska et al., 1967). The lactone form, hydroxyparaconic acid, is also produced.

Plasmid expressing genes necessary for itaconic conversion to itatartaric is transformed into an itaconic overproducing host. For example, Aspergillus and Ustilago strains are used as the host, specifically Aspergillus terreus, Aspergillus niger, Ustilago cynodontis, or Ustilago maydis. The itaconic oxidase activity occurs naturally from the wild type enzyme, from overexpression of the wild type gene, or from expression of mutant itaconic oxidase gene. An engineered E coli that overproduces itaconic acid, as described in Vuoristo et al 2014, could be transformed with the itaconic oxidase gene to produce itatartaric acid.

Itaconic acid oxidase activity can be detected using any method known in the art. For example, the assay described in Geiser et al can be used to determine itaconic oxidase activity by detected the product itatartaric acid via HPLC assay.

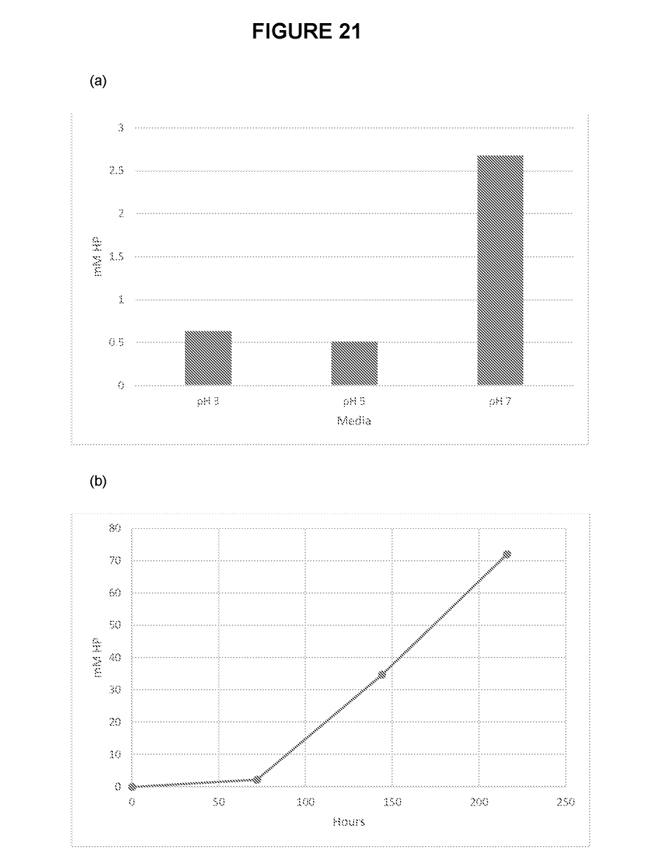

Ustilago maydis and Aspergillus terreus were grown in defined media for up to 9 days at 30° C. The growth media consisted of 120 g glucose, 1 g urea, 0.2 g KH₂PO₄, 1 g MgSO₄*7H₂O, 1 g yeast extract, 1 mL of 1000× trace metal solution per 1 liter adjusted to the indicated pH. The 1000× trace metal solution was made by addition of 0.125 g ZnSO4 and 1.25 g FeSO₄*7H₂O to 250 mL water. U. maydis was grown in pH 3, pH 5, and pH 7 medias, while A. terreus was grown in pH 3 media. Time points were taken approximately every 24 hours, and the supernatant was analyzed via H PLC. Itatartaric acid was observed to be predominantly present in its lactone form, hydroxyparaconic acid (HP). Levels of HP product were estimated by comparison with different amounts of synthesized ITT/HP standard. Both Ustilgo maydis and Aspergillus terreus produced HP (FIGS. 21(a) and (b)).
Aprai. ltaconic oxidase: an enzyme from an ultraviolet-induced mutant of Aspergillus terreus. Nature, 1958, 182, 661-662.
Arpai. Ultraviolet-induced mutational changes in enzyme activity of Aspergillus terreus. Journal of Bacteriology, 1959, 78, 153-158.
Geiser, Wiebach, Wierckx, and Blank. Prospecting the biodiversity of the fungal family Ustilaginaceae for the production of value-added chemicals. Fulgal Biology and Biotechnology 2014, 1:2.
Guevarra and Tabuchi. Accumulation of ltaconic, 2-hydroxyparaconic, itatartaric, and malic acids by strains of the genus Ustilago. Agric. Biol. Chem. 1990, 54 (9), 2353-2358.
Guevarra and Tabuchi. Production of 2-hydroxyparaconic and itatartaric acids by Ustilago cynodontis and simple recovery process of the acids. Agric. Biol. Chem., 1990, 54 (9), 2359-2365.
Jakubowska and Metodiewa. Studies on the metabolic pathway for itatartaric acid formation by Aspergillus terreus II. Use of (−)-citramalate, citraconate and itaconate by cell-free extracts. Acta Microbiologica Polonica Ser. B 1974, Vol. 6 (23), No. 2, 51-61.
Jakubowska, Oberman, Makiedonska, and Florianowicz. The itatonic and itatartaric acid formation by uv- and gamma-irradiated isolates of Aspergillus terreus NRRL 1960. 1967, 16(1), 53-68.
Vuuoristo et al. Metabolic engineering of itaconate production in Escherichia coli. Appl Microbiol Biotechnol, July 2014.

### Example 5—Construction of Recombinant Microorganism for Production of Alpha (Hydroxymethyl) Maleic Acid Starting from Alpha (Hydroxymethyl) Malic Acid

In addition to the DNA fragments listed in Examples 2 and 3, the DNA fragment encoding a dehydratase (FIG. 4, row C) is included. As shown generally in FIG. 3 and more specifically in FIG. 9 step C, dehydratase activity is increased in the recombinant microorganism through the introduction of recombinant nucleic acid sequences. Specifically, the dehydratase gene is aconitate hydratase from E. coli (EC 4.2.1.3). The resulting plasmid that successfully transcribes all pathway genes for production of alpha (hydroxyl methyl) maleic acid starting from alpha (hydroxyl methyl) malic acid is transformed into the organisms described in Examples 2, 3, and 4.

### Dehydratase Assay

E. coli optimized genes encoding dehydratases are synthesized and cloned into pTrcHisA (Life Technologies (formerly Invitrogen)). Dehydratase candidates are found in Table I. Plasmids containing the optimized synthase genes are transformed into BL21 E. coli cells. Empty plasmid pTrcHisA are also transformed as a negative control. For expression and characterization experiments, shake flasks containing 40 mL TB are inoculated at 5% from overnight cultures. Flasks are incubated at 30° C. at 250 rpm shaking for 2 hours, then protein production is induced with 0.2 mM IPTG and incubated for 4 more hours at 30° C. while shaking. Cells are harvested by centrifugation and pellets are stored at −80° C.

TABLE I
Dehydratase candidates.

| Gene | Organism | GenBank number |
|---|---|---|
| AcoA | Aspergillus nidulans | AAN61439 |
| Aco1 | Yarrowia lipolytica | AAT92542 |
| Aco1 | Saccharomyces cerevisiae | AAA34389 |
| Aco2 | Saccharomyces cerevisiae | CAA54757 |
| Leu2 | Saccharomyces cerevisiae | CAA27459 |
| TthacAB | Thermus thermophilus | BAA74762, BAA74763 |
| Aco | Sulfolobus acidocaldarius | AEG71149 |
| Aco | Sus scrofa | AAA30987 |
| AcnA | E. coli | CAA42834 |
| AcnB | E. coli | AAC73229 |
| AcoA | Aspergillus fumigatus | EAL89133 |

Activity of dehydratase candidates is assessed with an in vitro assay using the conversion of a single bond in the alpha substituted malic substrate to a double bond in the alpha substituted maleic product measured at 235 nm with a UV-spectrometer. The enzyme activity is tested using either no substrate or the alpha substituted malic as the substrate. The formation of the double bond causes an increase in absorption at 235 nm. The reaction can also be tested in the opposite direction, double bond to single bond, which results in a decrease in absorption at 235 nm. Either forward or reverse will give information to be able to calculate activity of the dehydratase candidate for the desired reaction. Unless otherwise specified, all chemicals are purchased from Sigma-Aldrich Chemical Company, St. Louis, Mo.

Cells are lysed using mechanical disruption using a BeadBeater (BopSpec products, Bartlesville, Okla.) using the manufacturer's instructions. The cell lysate is partially clarified by centrifugation (14,000G for 5 minutes). Protein concentrations of the resulting clarified lysates are measured via BioRad total Protein assay using the manufacturer's instructions. Lysates are normalized by protein concentration in 100 mM TAPS buffer. The normalized lysates are diluted 1 to 10 in 100 mM TAPS buffer. 10 μl of lysate was added to each well for the 96-well plate assay. Each condition was performed in triplicate.

The reaction mixture contains 100 mM TAPS buffer pH 6.8, 100 mM KCl, 100 mM substrate alpha (hydroxymethyl) maleic acid. The dehydratase lysates are incubated in the presence of 1 mM ammonium ferrous sulphate and 5 mM DTT to reconstitute the iron-sulfur cluster of the enzyme for 30 minutes. To start the reaction, 90 μl of reaction mix is added to each well already containing 10 μl lysate. The reactions in these microplates are monitored at 235 nm. Readings are taken every 9 seconds for 10 minutes and the data is used to calculate activities of each enzyme. Background absorbance is measured by the same reaction with no substrate present are subtracted.

The same reactions are allowed to incubate overnight at 30° C. The samples are boiled for 5 min at 100° C. to denature the protein. The samples are centrifuged to remove the protein debris and the resulting supernatant is analyzed by HPLC to measure formation of the desired product.

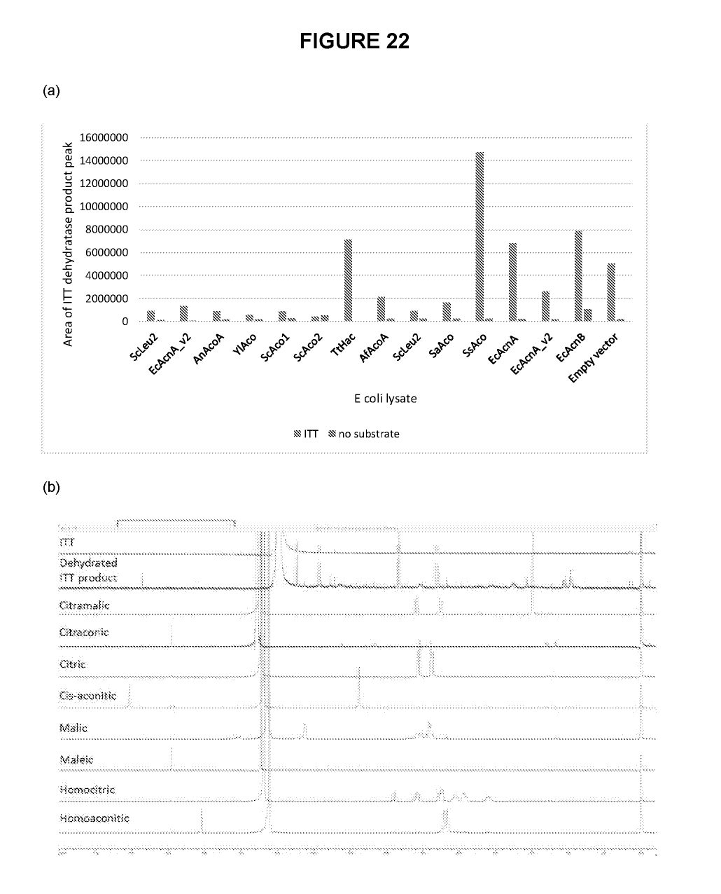

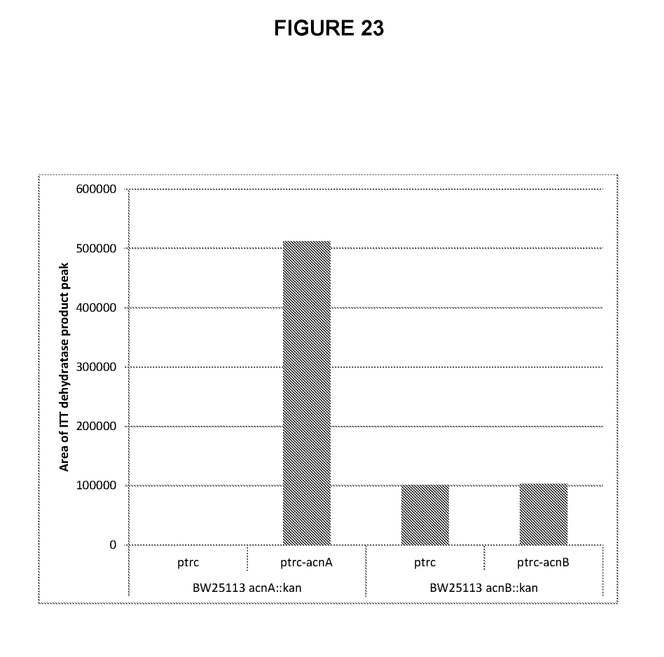

The lysates were incubated with or without a known amount of synthesized ITT overnight at 30° C., and then the samples were analyzed by HPLC. A peak was observed to noticeably increase when E coli cells were incubated in the presence of ITT (FIG. 22(a)). Samples were submitted for NMR analysis. The results indicate that a product was being formed in only the lysates in which ITT substrate had been added. Comparison to structural analogs, such as homocitrate and its dehydrated form homoaconitate, as well as citrate to cis-aconitate, citramalic to citraconic, etc., confirmed the presence of a peak characteristic of a dehydration product (FIG. 22(b)). The results suggest that an endogenous E. coli dehydratase is able to catalyze the ITT dehydration reaction, as indicated by presence of the dehydration product in empty vector control cells. Studies with knockout E coli strains demonstrated that E coli aconitase acnA is capable of producing the observed dehydration product (FIG. 23).

In FIG. 23, are shown the levels of dehydratase product as compared to the no substrate control in E coli cells with either acnA or acnB deleted, expressing either empty vector (ptrc) or plasmids expressing endogenous E coli dehydratases, acnA or acnB. The indicated lysate was incubated with or without itatartaric acid (ITT) present overnight at 30° C. The samples were analyzed by HPLC.

### Example 6—Construction of Recombinant Microorganism for Production of Alpha (Hydroxymethyl) Fumaric Acid Starting from Alpha (Hydroxymethyl) Maleic Acid

In addition to the DNA fragments listed in Example 5 the DNA fragment encoding an isomerase (FIG. 4, row F) is included. As shown generally in FIG. 3 and more specifically in FIG. 9 step F, isomerase activity is increased in the recombinant microorganism by through the introduction of a recombinant nucleic acid sequence. Specifically, the isomerase gene is selected from cis-trans isomerase from Pseudomonas putida (EC 5.2.1.1) and prpF from Shewenella oneidensis (Grimek et al, 2003). A third candidate is Adi1 (UMAG_11777) from Ustilago maydis (Geiser et al, 2016). The resulting plasmid that successfully transcribes all pathway genes for production of alpha (hydroxymethyl) fumaric acid starting from alpha (hydroxymethyl) maleic acid is transformed into the organism described by Example 5.

In another iteration, the DNA fragment encoding trans-homoaconitate synthase, aksA, from Methanosaeta thermophile or Methanococcus jannashii is included. Expression of aksA produces alpha (hydroxymethyl) fumaric acid from hydroxypyruvate (Howell et al, 1998).

The addition of a DNA fragment encoding an alpha (hydroxymethyl) fumaric acid transporter increases production alpha (hydroxymethyl) fumaric acid. Specifically, the transporter gene is fumaric transport gene, ydbH, from Bacillus subtillis (Asai et al, 2000).
Asai, Baik, Kasahara, Moriya, and Ogasawara. Regulation of the transport system of C4-dicarboxylic acids in Bacillus subtilis. Microbiology, 2000. 143, 263-271.
   Geiser, Przybilla, Friedrich, Buckel, Wierckx, Blank, and Bolker. Ustilago maydis produces itaconic acid via the unusual intermediate trans-aconitate. Microbiology Biotechnology, 2016. 9,116-129.
Grimek and Escalante-Semerena. The acnD genes of Shewenella oneidensis and Vibrio cholera encode a new Fe/S-dependent 2-methylcitrate dehydratase enzyme that requires prpF function in vivo. Journal of bacteriology, January 2004, p. 454-462.
Howell, Harich, Xu, and White. A-Keto acid chain elongation reactions involved in the biosynthesis of coenzyme B (7-mercaptoheptanoyl threonine phosphate) in methanogenic arches. Biochemistry, 1998. 37, 10108-10117.

### Example 7—Construction of Recombinant Microorganism for Production of Alpha (Hydroxyethyl) Malic Acid Utilizing the Spacer Pathway in a Serine Overproducing Microorganism

A serine producing organism is described in Example 2. In addition to the DNA fragments listed in Examples 2, 3, 4, and 5, the DNA fragments encoding a hydratase (FIG. 4, row D), a dehydrogenase (FIG. 4, row E), a synthase (FIG. 4, row B), a dehydratase (FIG. 4, row C) are included. As shown generally in FIG. 3 and more specifically in FIG. 9 steps B, C, D, and E, hydratase, dehydrogenase, synthase, and dehydratase activity is increased in the recombinant microorganism through the introduction of recombinant nucleic acid sequences. Specifically, the hydratase is homoaconitate hydratase from Schizosaccharomyces pombe (EC 4.2.1.36), the dehydrogenase is homoisocitrate dehydrogenase from Methanocaldococcus jannaschii (EC 1.1.1.87), the synthase is homocitrate synthase from Schizosaccharomyces pombe (2.3.3.14), and the dehydratase is aconitate hydratase from E. coli (4.2.1.3). Engineered mutants of these enzymes are constructed to increase specificity to the intermediates of the spacer pathway.

The resulting plasmid that successfully transcribes all pathway genes for production of alpha (hydroxyethyl) malic acid utilizing the ‘spacer pathway’ in a serine overproducing microorganism is transformed into the organisms described in Examples 2, 3, 4, and 5.

Additionally, expression of a DNA fragment encoding an alpha (hydroxyethyl) malic acid transporter improves production of alpha (hydroxyethyl) malic acid. Specifically, the transporter gene is selected from malic acid transport genes, tehA from E coli (UNIPROT E0IVN4), mael from S. pombe (Saayman et al, 2000), and ykxJ from Bacillus subtilis (Krom et al, 2001), or homologs thereof.
Krom, Aardema, and Lolkema. Bacillus subtilis YxkJ is a secondary transporter of the 2-hydroxycarboxylate transporter family that transports L-malate and citrate. J Bacteriol, 2001 October; 183(20):5862-9.
Saayman, van Vuuren, van Zyl, and Viljoen-Bloom. Differential uptake of fumarate by Candida utilis and Schizosaccharaomyces pombe. Appl Microbiol Biotechnol, 2000. 54: 792-798.

### Example 8—Construction of a Recombinant Microorganism for Production of Alpha (2-Hydroxypropyl) Malic Acid

The microorganism used for production of alpha (2-hydroxypropyl) malic acid can be selected from fungi, including yeast and filamentous fungi as well as bacteria. Homothreonine can be produced using the spacer pathway, described in Example 7, utilizing a threonine overproducing microorganism. In another iteration, expression of ilvA, leuA, leuCD, and leuB results in production of the intermediate 4-hydroxy-2-oxo-pentanoic acid (Shen and Liao). This iteration is utilized in a threonine overproducing strain as described in the review by Adrio and Demain. In instances were bacterial production is desired, E coli or Serratia marcencens can be used as a starting strain for subsequent genetic engineering steps. Similarly, the microorganism described in Ramos and Calderon can be used as a starting strain for subsequent genetic engineering steps in instances were eukaryotic production is desired.

In addition to the above example of a homothreonine over-expressing cell, the intermediate 4-hydroxy-2-oxo-pentanoic acid is produced through several alternative methods. In one iteration, expression of pyruvate aldolase (EC 4.1.3.39) produces the intermediate 4-hydroxy-2-oxo-pentanoic acid (Manjasetty et al).

In a homothreonine over-expressing strain, the DNA fragments encoding transaminase (FIG. 4, row A) and synthase (FIG. 4, row B) are cloned into an expression vector. Gene candidates and their sequences are identified in FIG. 4, far right column. As shown generally in FIG. 3 and more specifically in FIG. 10 steps A and B, transaminase and synthase activity are increased in the recombinant microorganism by through the introduction of recombinant nucleic acid sequences. Specifically, the transaminase gene is branched-chain-amino-acid transaminase from Schizosaccharomyces pombe (EC 2.6.1.42) and the synthase is 2-isopropylmalate synthase from Arabidopsis thaliana (2.3.3.13). The resulting plasmid successfully transcribes all pathway genes.

Additionally, expression of a DNA fragment encoding an alpha (2-hydroxypropyl) malic acid transporter improves production of alpha (2-hydroxypropyl) malic acid. Specifically, the transporter gene is selected from isopropylmalic acid transport gene, Oac1P, from S cerevisiae (Marobbio et al, 2008), or homologs thereof.
Adrio and Demian. Recombinant organisms for production of industrial production. Bioengineered Bugs, March/April 2010, 1:2, 116-131.
Manjasetty, Powlowski, and Vrielink. Crystal structure of a bifunctional aldolase-dehydrogenase: sequestering a reactive and volatile intermediate. PNAS, 2002, vol. 100, no. 12.
Marobbio, Giannuzzi, Paradies, Pierri, and Palmieri. a-isopropylmalate, a leucine biosynthesis intermediate in yeast, is transported by the mitochondrial oxaloacetate carrier. J Biol Chem. 2008. 283(42): 28445-28453.
Ramos and Calderon. Overproduction of threonine by Saccharaomyces cerevisiae mutants resistant to hydroxynorvaline. App and Environ Microb, May 1992, p. 1677-1682.
Shen and Liao. Metabolic engineering of Escherichia coli for 1-butanol and 1-propanol production via the keto-acid pathways. Metabolic Engineering, 2008, 10: 312-320.

### Example 9—Construction of Recombinant Microorganism for Production of Alpha Substituted 3-Hydroxypropionic Acid

In addition to the DNA fragments listed in Examples 2, 3, and 4 the DNA fragments encoding dehydratase (FIG. 6, row A), hydratase (FIG. 6, row B), reductase (FIG. 6, row C), decarboxylase (FIG. 6, row D), and aldehyde reductase (FIG. 6, row E) are cloned into an expression vector. Gene candidates and their sequences are indicated in FIG. 6, far right column. Specifically, the dehydratase gene is aconitate hydratase from E. coli (EC4.2.1.3), and the hydratase gene is homoaconitate hydratase from S. pombe (EC 4.2.1.36), the reductase gene is malate dehydrogenase from S. cerevisiae (EC1.1.1.37), the decarboxylase gene is branched chain 2-oxoacid decarboxylase from S. cerevisiae (EC 4.1.1.72), and the aldehyde reductase gene is aldehyde reductase from S. cerevisiae (EC 1.1.1.21). The resulting plasmid that successfully transcribes all pathway genes is transformed into a host organism as described in Example 2, 3, or 4.

Specific examples of step A, B, and C are illustrated in the leucine synthesis pathway in which the alpha substituted malic acid is 2-ispropylmalic acid. The enzyme 3-isopropylmalate dehydratase performs both the hydration and dehydratase steps to result in 3-isopropylmalate. The enzyme 3-isopropylmalate dehydrogenase provides the reductase action illustrated in step C. These enzymes are present in many species including yeast (Hsu and Kohlhaw). For an example of step D, decarboxylase, the enzyme may be a 2-keto acid decarboxylase. Multiple 2-oxo acid decarboxylases exist in nature and within a single organism with different specificities that can be utilized (Romagnoli et al.). Engineering of 2-keto acid decarboxylases to change specificity has also been demonstrated, for example by Zhang et al. An example of the reductase (step E) can be an alcohol dehydrogenase, adh. Aldehyde reductase/alcohol dehydrogenase genes have been demonstrated to have a wide specificity, for example in E. coli by Atsumi et al.
Atsumi, et al. Engineering the isobutanol biosynthetic pathway in Escherichia coli by comparison of three aldehyde reductase/alcohol dehydrogenase genes. Appl Microbiol Biotechnol 2010, 85:651-657.
Hsu and Kohlhaw. Leucine biosynthesis in Saccharomyces cerevisiae. JBC 1979, Vol 255, No. 15, pp. 7255-7260.
Romagnoli et al. Substrate specificity of thiamine pyrophosphate-dependent 2-oxo acid decarboxylases in Saccharomyces cerevisiae. Appl Environ Microbiol 2012, 78(21):7538.
Zhang et al. Expanding metabolism for biosynthesis of non-natural alcohols. PNAS, December 2008, vol. 105, no. 52, 20653-20658.

### Example 10—Fermentation

Fed-batch fermentation is performed in a 2 L working capacity fermenter. Temperature, pH and dissolved oxygen are controlled by PID control loops. Temperature is maintained at 37° C. by temperature adjusted water flow through a jacket surrounding the fermenter vessel at the growth phase, and later adjusted to 27° C. when production phase started. The pH is maintained at the desired level by the addition of 5 N KOH and 3 N H₃PO₄. Dissolved oxygen (DO) level is maintained at 20% of air saturation by adjusting air feed as well as agitation speed.

Inoculant is started by introducing a single colony picked from an LB agar plate into 50 mL TB medium. The culture is grown at 37° C. with agitation at 250 rpm until the medium is turbid. Subsequently a 100 mL seed culture is transferred to fresh M9 glucose medium. After culturing at 37° C. and 250 rpm for an additional 10 h, an aliquot (50 mL) of the inoculant (0D600=6-8) is transferred into the fermentation vessel and the batch fermentation was initiated. The initial glucose concentration in the fermentation medium is about 40 g/L.

Cultivation under fermentor-controlled conditions is divided into two stages. In the first stage, the airflow is kept at 300 ccm and the impeller speed is increased from 100 to 1000 rpm to maintain the DO at 20%. Once the impeller speed reaches its preset maximum at 1000 rpm, the mass flow controller starts to maintain the DO by oxygen supplementation from 0 to 100% of pure O₂.

The initial batch of glucose is depleted in about 12 hours and glucose feed (650 g/L) is started to maintain glucose concentration in the vessel at 5-20 g/L. At OD600=20-25, IPTG stock solution is added to the culture medium to a final concentration of 0.2 mM. The temperature setting is decreased from 37 to 27° C. and the production stage (i.e., second stage) is initiated. Production stage fermentation is run for 48 hours and samples are removed to determine the cell density and quantify metabolites. Production of specific products is measured by GS/MS.

### Example 11—Separation

Fermentation broth containing alpha (hydroxymethyl) malic acid, or itatartaric acid, produced from cell cultures as described in Examples 2, 3, and 4 is treated using the procedure as described in Guevarra and Tabuchi et al., 1990, to separate the desired product. Fermentation broth is filtered to remove cells, then concentrated to a syrup. The resulting syrup is heated to 70° C. for 6 hours under reduced pressure to catalyze lactonization of alpha (hydroxymethyl) malic acid, or itatartaric acid, to the cyclized form, hydroxyparaconic (HP). A solid mass results that is dissolved in heated ethyl acetate under vigorous agitation. The solvent layer is separated, then concentrated and dried until a crystalline mass is formed. Further purification of HP is performed by crystallization with ethyl acetate and chloroform. The purity of the final product is analyzed via HPLC. Itatartaric sodium salt is prepared from the recrystallized HP by being dissolved in cold H₂O and titrated with 0.05n NaOH. The solution is heated for 10 minutes in a boiling water bath, then concentrated under reduced pressure, and finally, dried in a 105° C. oven. The purity of the final product is analyzed via HPLC.

Other alpha substituted malic acid intermediates, including homocitrate, are separated using methods developed for various carboxylic acids. Such methods include separation using anion exchange, ultra-filtration, distillation, electro-dialysis, reverse osmosis, and various extraction methods as reviewed in Kumar and Babu 2008.
Guevarra and Tabuchi. Production of 2-hydroxyparaconic and itatartaric acids by Ustilago cynodontis and simple recovery process of the acids. Agric. Biol. Chem., 1990, 54 (9), 2359-2365.
Kumar and Babu. Process intensification for separation of carboxylic acids from fermentation broths using reactive extraction. Journal on Future Engineering & Technology, Vol. 3(3), pp 19.26.

### Example 12—Conversion of Homocitric Acid to 2-Methylene Glutaric Acid

Conversion of functionalized alpha substituted C4 dicarboxylic acid to a functionalized alpha substituted acrylic acid was performed by contacting a Pt-based catalyst with sodium homocitric acid to produce 2-methylene glutaric acid.

### Materials and Methods

The experiment was performed using 5% Pt/Al₂O₃ and addition of 0.1N NaOH base. The catalyst loading was 2.5 mol % (calculated on dry powder basis of Pt metal), and the solvent used was 0.1N NaOH. The reaction time was 16 hours under 450 psi of N₂ at temperature of 180° C. Another reaction was carried out in pure water with Cu-based catalyst (50 mol % based on Cu-metal) in an autoclave using sod. homocitrate salt under 500 psi H2 gas.

The reaction products were analyzed using GC/MS (Agilent, 5975B, inert, XL, EI/CI). The evaluation of the catalyst was based on qualitative results of the GC/MS data. Other catalysts tested include 5% Pd/CaCO₃, 5% Pd/BaSO₄, Cu-0860 (pre-reduced) BASF, Cu-0860 (unreduced) BASF, Cu/Zn/AI (pre-reduced). In all of the Pt and Pd supported catalysts, the metal loadings were 2.5 mol % except the Cu-based catalysts which were used 50 wt % on dry metal basis for this set of reaction. Commercial catalysts were activated just prior to use. Catalyst activation was performed in the CCRI High-Throughput facility using Symyx high throughput reactor by the following protocol:

a. Anneal at 180° C. under 400 psi of N₂ for 2 hr,

b. Anneal at 180° C. under 200 psi of H₂ for 2 hr.

The reactions were carried out using Symyx High Throughput Module (Symyx Discovery Tools). In a typical experiment, sodium homocitrate (0.12 mmol, 0.032 g) was added to 1 mL 0.1N NaOH in glass vials equipped with magnetic stir bar. The substrate was allowed to dissolve in aqueous 0.1N NaOH by stirring at room temperature. The resulting solution was then added to pre-activated catalysts (2.5 mol % Pt or Pd or 50 wt % Cu dry metal basis) in a vial placed on 24 wells plate and loaded on the Core Module. The reaction mixture was pressurized with 450 Psi N₂ gas and heated at 180° C. temperature with continuous stirring for 16 h. After the reaction, 200 μL of the supernatant from the reaction vial was transferred into an oven dry vial and allowed to dry completely in a freeze drier.

The dried sample was used for derivatization in order to commence GC-MS analysis.

500 μL of methanol and one drop of sulfuric acid were added to fully dried samples (200 μL of the supernatant from the reaction vial), then sealed, stirred and heated samples at 70° C. for 90 minutes. After cool-down, about 30-40 mg of solid sodium bicarbonate were added manually to samples using a mg-scoop and stirred 5-10 minutes. 300 μL of brine and 300 μL of water were added and the resulting mixture was stirred another 5 minutes. 600 μL of ACS-grade ethyl acetate was added, then the samples were mixed well to ensure full contact of the two phases and establish partition equilibrium (phases separated well enough after sitting for 5-10 minutes). Finally 200 μL was removed off the top organic phase and diluted to 1000 μL for GC-MS analysis (with MeOH).

### Results and Discussion

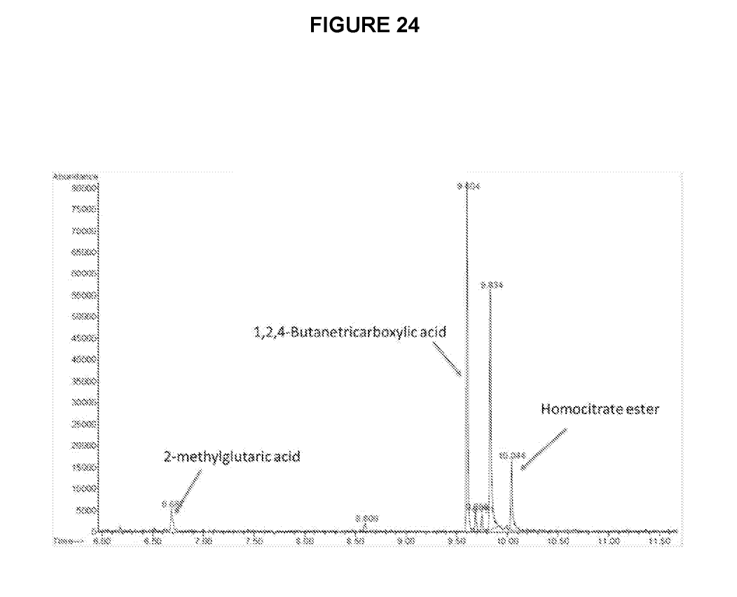

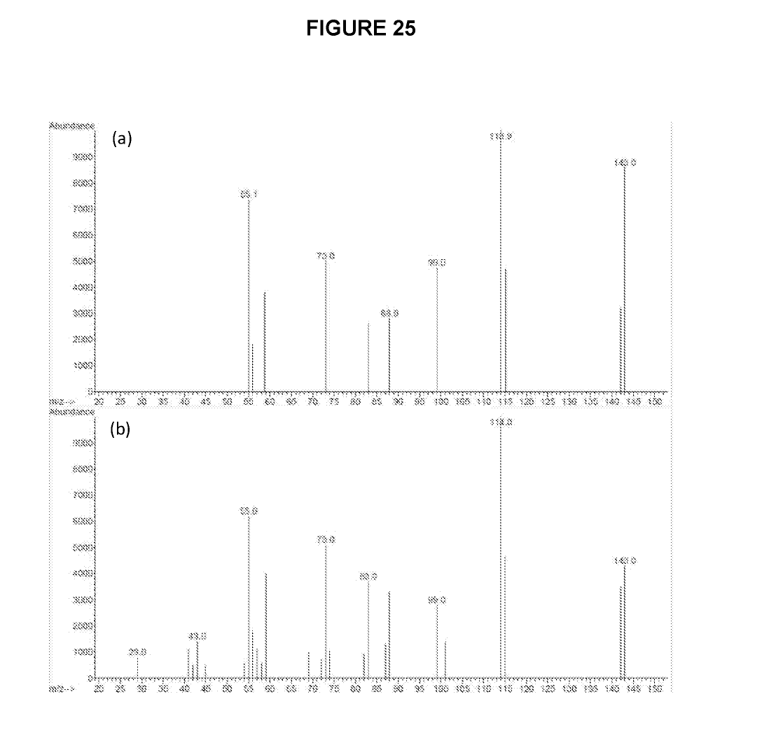

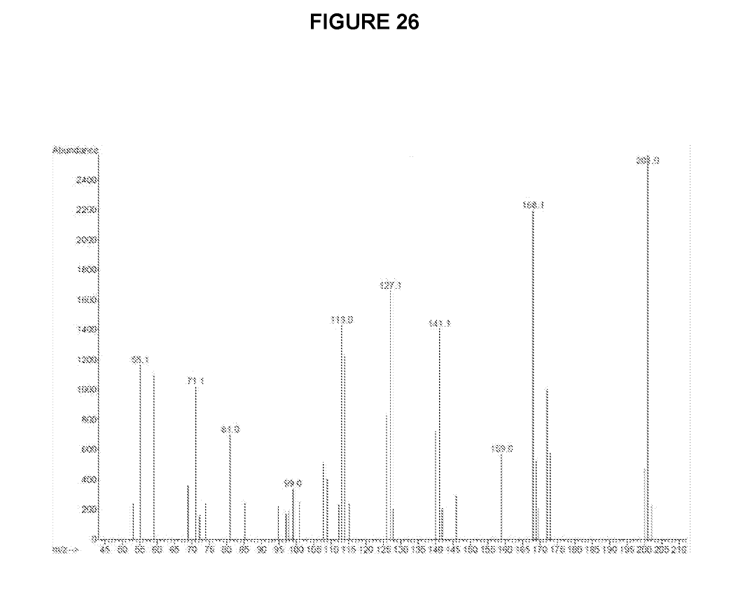

Homocitric acid trisodium salt was contacted with various metal catalysts to promote the conversion to the alpha substituted acrylic acid, specifically 2-methylene glutaric acid. Results of the experiment in which sodium homocitrate was contacted with 5% Pt/Al₂O₃ are shown in the chromatogram of FIG. 24. Known peaks include the starting material, homocitrate ester, and the hydrogenated form of the desired acrylate product, 2-methylglutaric acid. Other identified products include 1,2,4-butanetricarboxylic acid at 9.6 mins and lactone ester at 9.834 mins along with the starting homocitrate after methylester derivatization at retention time 10.044 mins. Mass spectra results for some of these peaks are shown in FIG. 25(a), FIG. 25(b) and FIG. 26.

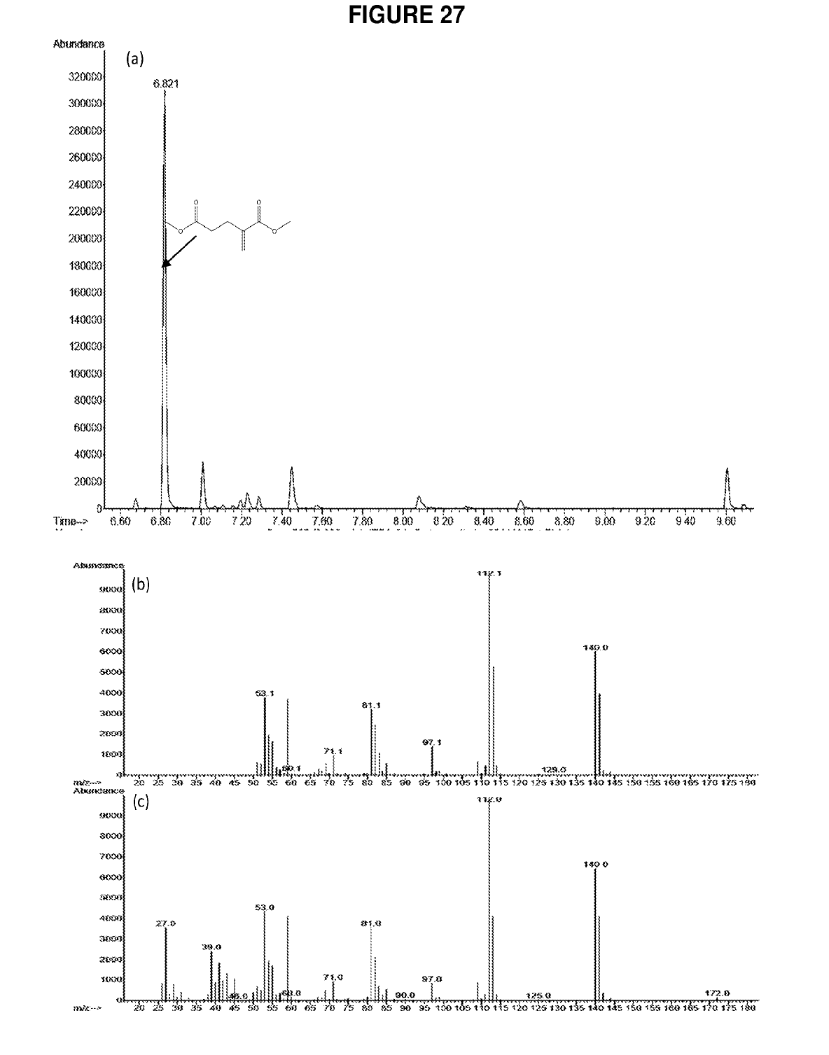

When the reaction was carried out in pure water with Cu-based catalyst (50 mol % based on Cu-metal) in an autoclave using sodium homocitrate salt under 500 psi H₂ gas, GC-MS chromatogram showed the formation of 2-methylene glutaric acid as the main peak. FIG. 27(a) shows the GC-MS chromatogram of autoclave (5 mL) reaction with Cu-based catalyst with sodium homocitrate in water under 500 psi H₂ gas pressure, while FIG. 27(b)/(c) shows the mass spectra of the 2-methylene glutaric acid produced.

### Example 13—Conversion of 2-Isopropylmalic Acid to Alpha-Isopropyl Acrylic Acid

Conversion of functionalized alpha substituted C4 dicarboxylic acid to a functionalized alpha substituted acrylic acid was successfully carried out by contacting a Cu-based catalyst with 2-isopropylmalic acid to result in alpha-isopropyl acrylic acid.

### Materials and Methods

The experiment was performed using Cu catalyst under either H₂ or N₂. The catalyst loading was 50 wt % (calculated on dry powder basis), and the solvent used was H₂O. The reaction time was 16 hours under 450 psi of H₂ or N₂, as indicated, at temperature of 180° C. The reaction products were analyzed using GC/MS (Agilent, 5975B, inert, XL, EI/CI). The evaluation of the catalysts was based on qualitative results of the GC/MS data.

Commercial CuO catalysts were activated just prior to use. Catalyst activation was performed in the CCRI High-Throughput facility using Symyx high throughput reactor by the following protocol:
a. Anneal at 180° C. under 400 psi of N₂ for 2 hr,
b. Anneal at 180° C. under 200 psi of H₂ for 2 hr.

The reactions were carried out using Symyx High Throughput Module (Symyx Discovery Tools). In a typical experiment, 2-isopropylmalic acid (0.12 mmol, 0.0213 g) was added to 1 mL H₂O in a glass vial equipped with magnetic stir bar. The substrate was allowed to dissolve in water by stirring at room temperature. The resulting solution was then added to pre-activated catalysts (50 wt %, 0.01065 g) in a vial placed on 24 wells plate and loaded on the Core Module. The reaction mixture was first pressurized either with 450 Psi H₂ or N₂ gas and heated at 180° C. temperature with continuous stirring for 16 h. After the reaction, 200 μL of the supernatant from the reaction vial was transferred into an oven dry vial and allowed to dry completely in freeze drier.

The dried sample was used for derivatization in order to commence GC-MS analysis. 500 μL of methanol and one drop of sulfuric acid were added to fully dried samples (200 μL of the supernatant from the reaction vial), then sealed, stirred and heated samples at 70° C. for 90 minutes. After cool-down, about 30-40 mg of solid sodium bicarbonate were added manually to samples using a mg-scoop and stirred 5-10 minutes. 300 μL of brine and 300 μL of water were added and the resulting mixture was stirred another 5 minutes. 600 μL of ACS-grade ethyl acetate was added, then the samples were mixed well to ensure full contact of the two phases and establish partition equilibrium (phases separated well enough after sitting for 5-10 minutes). Finally 200 μL was removed off the top organic phase and diluted to 1000 μL for GC-MS analysis (with MeOH).

### Results and Discussion

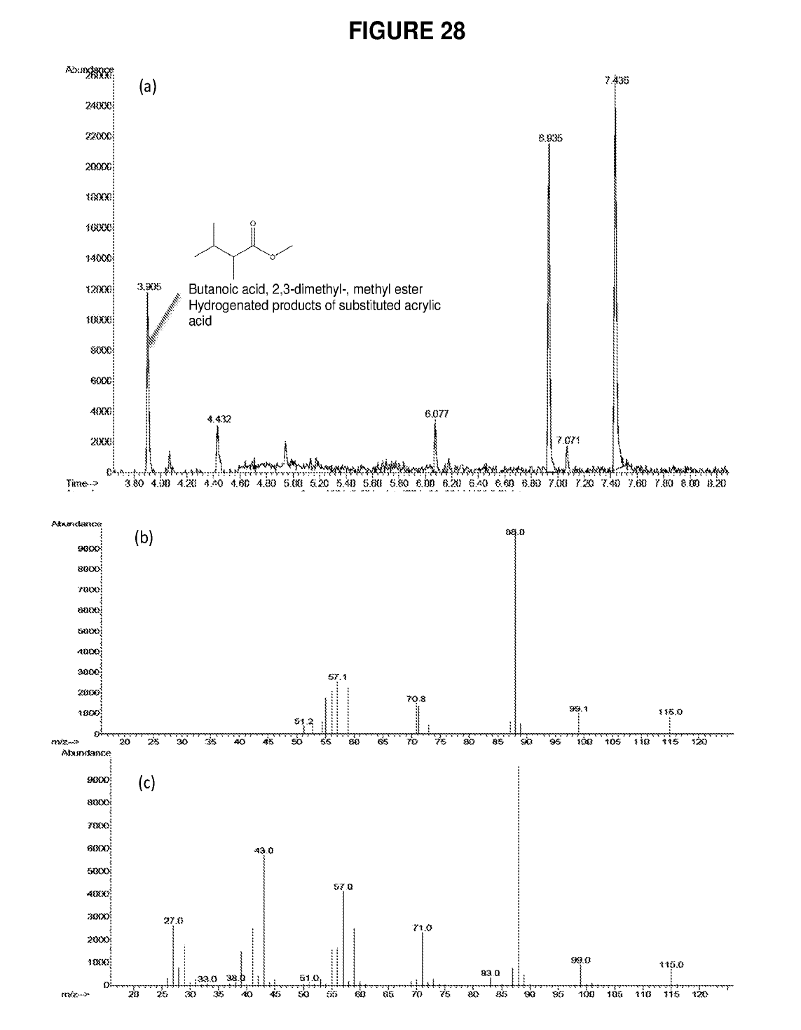

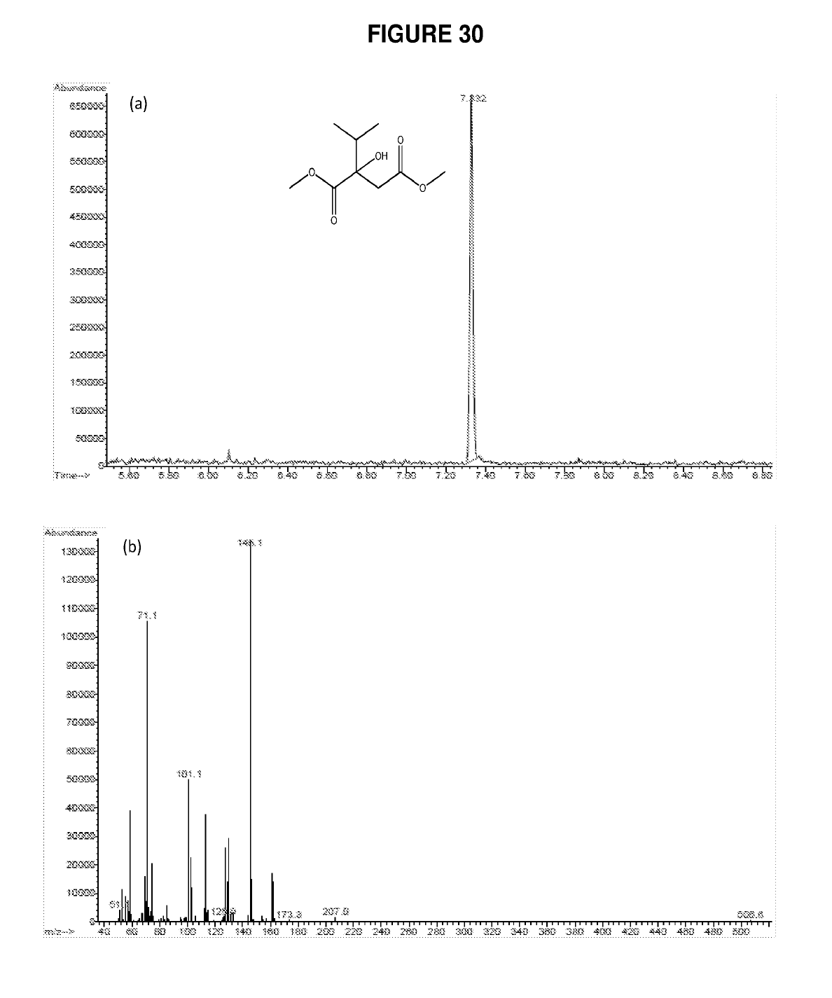

The starting material, 2-isopropylmalic acid, was contacted with the Cu-based catalyst to promote the conversion to alpha substituted acrylic acid, alpha-isopropyl acrylic acid. Results of the experiment in which 2-isopropylmalic acid was contacted with the Cu-based catalyst under H₂ are shown in the chromatogram of FIG. 28(a). Known peaks include butanoic acid 2,3-dimethyl, methyl ester- the hydrogenated product of the desired alpha substituted acrylic acid. The mass spectra of butanoic acid 2,3-dimethyl, methyl ester is shown in the mass spectra of FIG. 28(b)/(c). Another peak was determined to likely be the dehydrated followed by hydrogenated derivative of 2-isopropylmalic acid, dimethyl ester (1-methylethyl)-butanedioic acid. The peak at 7.435 minutes being the unreacted starting material, 2-isopropylmalic acid under the specified reaction conditions (Note: the retention time of 7.435 mins is slightly different than authentic sample at 7.329 min in FIG. 30(a) because the sample was analyzed before maintenance of the GC-column).

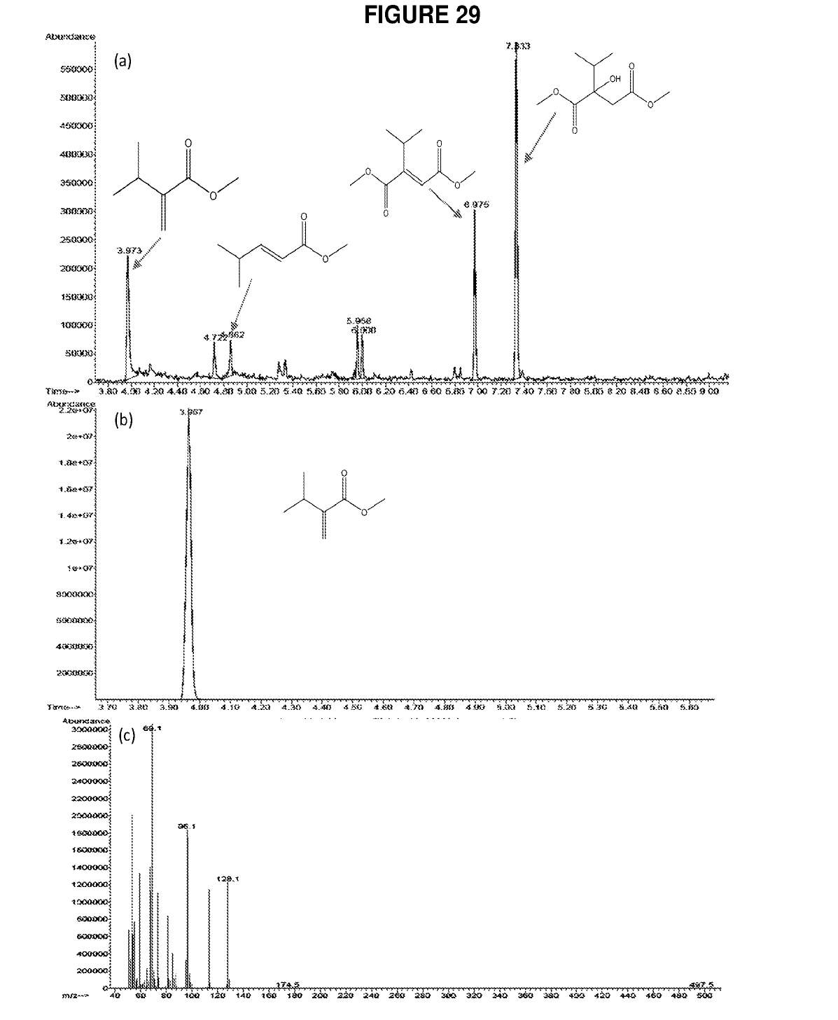

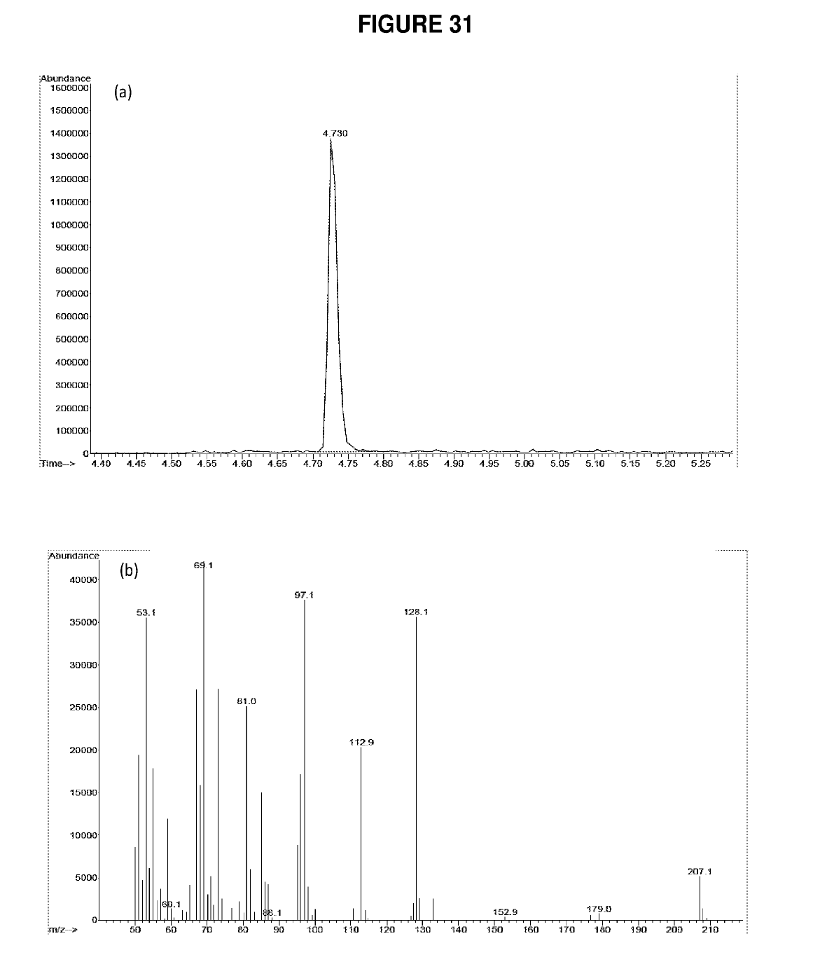

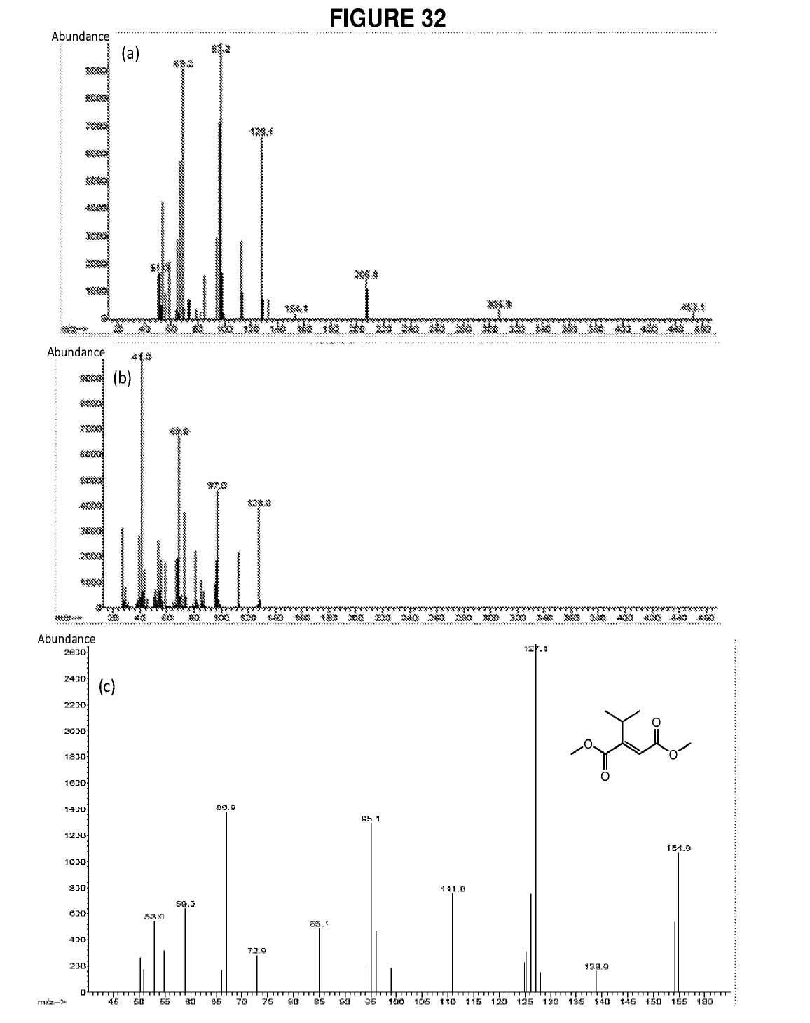

To prevent the hydrogenation of the methylene group of the desired alpha substituted acrylic acid, the reaction was repeated under N₂ in place of H₂. All other conditions were identical. The reaction resulted in production of the desired product, isopropyl acrylic acid (chromatogram of FIG. 29(a)). The mass spectrum of isopropyl acrylic acid is shown in FIG. 29(b)/(c). The spectrum of the starting material, 2-isopropyl malic acid is show in FIG. 30(a)/(b). Other peaks were identified as 4-methyl-2-pentenoic acid and 2-(1-methylethyl)-2-butenedioic acid (FIG. 31(a)/(b) and FIG. 32(c)).

### Example 14—Conversion of Hydroxyalkyl Alpha Substituted C4 Diacid to Hydroxyalkyl Alpha Substituted Acrylic Acid

Conversion of functionalized alpha substituted C4 dicarboxylic acid to a functionalized alpha substituted acrylic acid is carried out by contacting a Cu- or Pt-based catalyst with hydroxyalkyl alpha substituted C4 diacid to produce a hydroxyalkyl alpha substituted acrylic acid. Specifically the hydroxyalkyl alpha substituted C4 diacid is alpha (hydroxymethyl) malic acid and the product is alpha (hydroxymethyl) acrylic acid.

### Materials and Methods

The experiment is performed using either a Cu or Pt catalyst under N₂ or H₂. When the catalyst is Cu, catalyst loading is 50 wt % (calculated on dry powder basis), and the solvent used is H₂O. When the catalyst is 5% Pt/Al₂O₃, the catalyst loading is 2.5 mol % (calculated on dry powder basis), and the solvent used is 0.1N NaOH. For either catalyst condition, the reaction time is 16 hours under 450 psi of N₂ or H₂ at temperature of 180° C. The reaction is performed as described in examples 12 and 13. The reaction products are analyzed using GC/MS (Agilent, 5975B, inert, XL, EI/CI). The evaluation of the catalysts is based on qualitative results of the GC/MS data. The analytics is performed as described in examples 12 and 13.

### Results and Discussion

Hydroxyalkyl alpha substituted C4 diacid is contacted with a Cu-based or Pt-based catalyst to promote the conversion to hydroxyalkyl alpha substituted acrylic acid. Specifically the hydroxyalkyl alpha substituted C4 diacid is alpha (hydroxymethyl) malic acid and the product is alpha (hydroxymethyl) acrylic acid.

### Example 15—Conversion of Alpha (2-Hydroxyethyl) Malic Acid to Tulipalin

Conversion of the product of Example 7, alpha hydroxyethyl malic acid to a functionalized alpha substituted acrylic acid, alpha hydroxyethyl acrylic acid, is carried out by contacting a Cu- or Pt-based catalyst with hydroxyalkyl alpha substituted C4 diacid, alpha hydroxyethyl malic acid, to produce the hydroxyalkyl alpha substituted acrylic acid, alpha (2-hydroxyethyl) acrylic acid. The hydroxyalkyl alpha substituted acrylic acid product, alpha (2-hydroxyethyl) acrylic acid, is lactonized to result in tulipalin. As those familiar in the art will appreciate, lactonization is carried out using any strong acid catalyst such as sulfuric acid, hydrochloric acid, etc.

### Materials and Methods

The experiment is performed using either a Cu or Pt catalyst under N₂ or H₂. When the catalyst is Cu, catalyst loading is 50 wt % (calculated on dry powder basis), and the solvent used will be H₂O. When the catalyst is 5% Pt/Al₂O₃, the catalyst loading is 2.5 mol % (calculated on dry powder basis), and the solvent used is 0.1N NaOH. For either catalyst condition, the reaction time is 16 hours under 450 psi of N₂ or H₂ at temperature of 180° C. The reaction is performed as described in examples 12 and 13. The reaction products is analyzed using GC/MS (Agilent, 5975B, inert, XL, EI/C1). The evaluation of the catalysts is based on qualitative results of the GC/MS data. The analytics is performed as described in examples 12 and 13.

### Results and Discussion

Alpha (2-hydroxyethyl) C4 diacid is contacted with a Cu-based or Pt-based catalyst to promote the conversion to alpha-(2-hydroxyethyl)acrylic acid. The alpha (2-hydroxyethyl) acrylic acid product is lactonized to tulipalin.

### Example 16—Conversion of Alpha (2-Hydroxypropyl) Malic Acid to MeMBL

Conversion of the product of Example 8, alpha (2-hydroxypropyl) malic acid to a functionalized alpha substituted acrylic acid, alpha (2-hydroxypropyl) acrylic acid, is carried out by contacting a Cu- or Pt-based catalyst with the alpha substituted C4 diacid, alpha (2-hydroxypropyl) malic acid, to produce the alpha substituted acrylic acid, alpha (2-hydroxypropyl) acrylic acid. The alpha substituted acrylic acid product, alpha (2-hydroxypropyl) acrylic acid, is lactonized to result in MeMBL. As those familiar in the art will appreciate, lactonization is carried out using any strong acid catalyst such as sulfuric acid, hydrochloric acid, etc.

### Materials and Methods

The experiment is performed using either a Cu or Pt catalyst under N₂ or H₂. When the catalyst is Cu, catalyst loading is 50 wt % (calculated on dry powder basis), and the solvent used is H₂O. When the catalyst is 5% Pt/Al₂O₃, the catalyst loading is 2.5 mol % (calculated on dry powder basis), and the solvent used is 0.1N NaOH. For either catalyst condition, the reaction time is 16 hours under 450 psi of N₂ or H₂ at temperature of 180° C. The reaction is performed as described in examples 12 and 13. The reaction products is analyzed using GC/MS (Agilent, 5975B, inert, XL, EI/CI). The evaluation of the catalysts is based on qualitative results of the GC/MS data. The analytics is performed as described in examples 12 and 13.

### Results and Discussion

Alpha (2-hydroxypropyl) malic acid is contacted with a Cu-based or Pt-based catalyst to promote the conversion to alpha (2-hydroxypropyl) acrylic acid. The alpha (2-hydroxypropyl) acrylic acid product is lactonized to MeMBL.

### Example 17—Selection of Host for Engineering of Functionalized Alpha Substituted Acrylic Acids

Potential hosts for the described pathways to alpha substituted acrylic acid were identified by determining the tolerance to functionalized alpha substituted acrylic acid end-products as shown generally in FIGS. 1 and 3 and more specifically in FIGS. 9 and 10. The indicated functionalized alpha substituted acrylic acids, functionalized alpha substituted acrylic acid esters, and functionalized alpha substituted acrylic acid lactones were selected to assess tolerance of bacterial and eukaryotic hosts (Tables K and L).

Two eukaryotic strains, I. orientalis and S. cerevisiae, were tested for tolerance to the selected compounds at pH 3 and pH 5. The bacterial strains were grown at their individual optimal conditions, E coli and C. glutamicum at pH 8 and 30° C., and B. firmus and B. cohnii at pH 9 and 37° C. I. orientalis was grown in defined yeast media consisting of 5 g/L ammonium sulfate, 0.5 g/L magnesium sulfate heptahydrate, 3 g/L potassium phosphate monobasic, 10 g/L dextrose, 1 mL/L of 10% glycerol stock, 1 mL/L of 1000× trace. The 1000× trace stock solution contains 4 g/L ZnSO₄.7H₂O, 2 g/L FeSO₄.7H₂O, 1 g/L MnSO₄.H₂O, 0.2 g/L CuSO₄.5H₂O, and 0.8 mL/L H₂SO₄. S. cerevisiae was grown in buffered defined dextrose media consisting of 50 g/L dextrose, 5 g/L yeast extract, and 40 mL/L 25×DM salts. The 25×DM salt stock solution contains 125 g/L ammonium sulfate, 12.5 g/L magnesium sulfate heptahydrate, 75 g/L potassium phosphate monobasic, and 787.5 g/L water. The bacterial strains were grown in standard LB media consisting of 10 g/L bacto-tryptone, 5 g/L yeast extract, 10 g/L NaCl, and 20 g/L dextrose with addition of 20 g/L glucose.

Time points were taken over a period of at least 8 hours and up to 24 hours to calculate the rate of growth. Specific growth rate was determined by plotting the natural logarithm of cell number against time. Tolerance was determined by growth rate of cells in the presence of the compound as compared to in the absence of the compound. The scoring method is indicated in Table J. The results of the tolerance studies are shown in Tables K and L. The second column from the left denotes the maximum concentration of the indicated compound in which growth was detected. The results suggest that select bacterial hosts could be suitable hosts for production of alpha substituted acrylates, specifically E. coli for hydroxymethyl acrylate and C. glutamicum and B. firmus for hydroxyethyl acrylate and methyl hydroxyethyl acrylate.

TABLE J
Subjective system used to score effect of
specific chemical on cell growth rate.

| % Growth rate | |
|---|---|
| as compared to | |
| unchallenged | |
| cells | Score |
| 100-90% | +++ |
| 89-50% | ++ |
| 49-11% | + |
| 10-0% | − |

TABLE K
Tolerance of potential yeast hosts.

| | Max conc | I. orientalis | | S. cerevisiae | |
|---|---|---|---|---|---|
| Compound | (g/L) | pH 3 | pH 5 | pH 3 | pH 5 |
| Hydroxyethyl acrylate | 5 | + | + | + | + |
| Methyl hydroxyethyl | 5 | + | + | + | + |
| acrylate | | | | | |
| Hydroxymethyl acrylate | 20 | − | − | − | − |
| Methyl hydroxymethyl | 20 | − | − | − | − |
| acrylate | | | | | |
| Ethyl hydroxymethyl | 20 | − | − | − | − |
| acrylate | | | | | |
| MBL | 5 | − | − | − | − |
| MeMBL | 5 | − | − | − | − |

TABLE L
Tolerance of potential bacterial hosts.

| | Max conc | | C. | B. | B. |
|---|---|---|---|---|---|
| Compound | (g/L) | E. coli | glutamicum | cohnii | firmus |
| Hydroxyethyl acrylate | 20 | − | + | + | ++ |
| Methyl hydroxyethyl | 20 | + | ++ | nt | +++ |
| acrylate | | | | | |
| Hydroxymethyl acrylate | 20 | +++ | − | nt | nt |
| Methyl hydroxymethyl | 20 | − | − | nt | nt |
| acrylate | | | | | |
| Ethyl hydroxymethyl | | | | | |
| acrylate | | | | | |
| MBL | 5 | − | − | nt | nt |
| MeMBL | 5 | − | − | nt | nt |

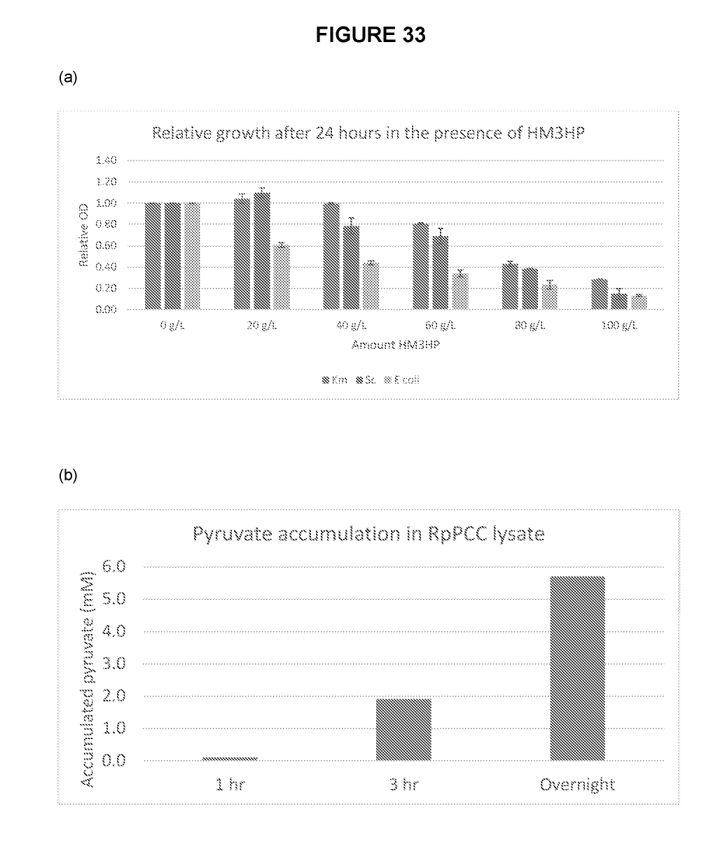

In one embodiment, alpha substituted 3-hydroxypropionic acid is produced by the host organism. In a specific embodiment, alpha hydroxymethyl 3-hydroxypropionic acid is produced by the host organism. To evaluate host organisms, the protocol described above was used. The relative OD at the end of 24 hours incubation at 30° C. was compared for Kluyveromyces marxianus, S. cerevisiae, and E coli in the presence of different amounts of hydroxymethyl-3HP (FIG. 33(a)). All three organisms were able to tolerate alpha-hydroxymethyl 3-hydroxypropionic acid up to at least 60 g/L. K. marxianus showed the best tolerance of the organisms tested for this compound.

It is understood that once a host organism is chosen the codons used in the synthetic genes are customized to that of the host cells.

### Example 18—Construction of Recombinant Microorganism for Conversion of Alpha (Hydroxymethyl) C4 Dicarboxylic Acid to Alpha (Hydroxymethyl) Acrylic Acid and Corresponding Esters

In addition to the DNA fragments listed in Example 5, the DNA fragment encoding decarboxylase (FIG. 12, row I) is cloned into an expression vector. Gene candidates and their sequences are shown in FIG. 12, far right column. Specifically, the decarboxylase gene is cis-aconitase decarboxylase, cadA, from Aspergillus niger (EC 4.1.1.6). The resulting plasmid that successfully transcribes all pathway genes is transformed into a recombinant microorganism that produces alpha (hydroxymethyl) maleic acid as described in Example 5 to result in alpha (hydroxymethyl) acrylic acid. The decarboxylase plasmid is also expressed in cells producing alpha (hydroxymethyl) fumaric acid as described in Example 6 to result in alpha (hydroxymethyl) acrylic acid.

Additionally, expression of a DNA fragment encoding an alpha (hydroxymethyl) acrylic acid transporter improves production of alpha (hydroxymethyl) acrylic acid. Specifically, the transporter gene is msfA encoding the putative Major Facilitator Superfamily protein from Aspergillus terreus (UNIPROT Q0C8L2).

### Decarboxylase Activity Assay

E coli optimized genes encoding decarboxylases are synthesized and cloned into pTrcHisA (Life Technologies (formerly Invitrogen)). Decarboxylase candidates are found in table M. Plasmids containing the optimized synthase genes were transformed into BL21 E. coli cells. Empty plasmid pTrcHisA is also transformed as a negative control. For expression and characterization experiments, shake flasks containing 40 mL TB are inoculated at 5% from overnight cultures. Flasks are incubated at 30° C. at 250 rpm shaking for 2 hours, then protein production is induced with 0.2 mM IPTG and incubated for 4 more hours or overnight at 30° C. while shaking. Cells are harvested by centrifugation and pellets were stored at −80° C.

Activity of decarboxylase candidates are assessed with an in vitro lysate assay whereas the acrylate product is detected using HPLC. The enzyme activity is tested using either no substrate or the alpha substituted maleic as the substrate. The acrylate product is detected using Benson organic acid column (300×7.8 mm, Part #2000-0 BP-OA) and run using 2 Benson columns in tandem, 4% acetonitrile+0.025 N sulfuric acid mobile phase. Unless otherwise specified, all chemicals are purchased from Sigma-Aldrich Chemical Company, St. Louis, Mo.

Cells are lysed using mechanical disruption using a BeadBeater™ (BopSpec products, Bartlesville, Okla.) using the manufacturer's instructions. The cell lysate is partially clarified by centrifugation (14,000G for 5 minutes). Protein concentrations of the resulting clarified lysates are measured via BioRad total Protein assay using the manufacturer's instructions. Lysates are normalized by protein concentration in 100 mM sodium phosphate buffer, pH 6.3.

The reaction mixture contains 100 mM Sodium phosphate buffer pH 6.3, 1 μl DTT, and 10 mM substrate alpha (substituted) maleic acid. The reactions are allowed to incubate overnight at 30° C. The samples are boiled for 5 min at 100° C. to denature the protein. The samples are centrifuged to remove the protein debris and the resulting supernatant is analyzed by HPLC to measure formation of the desired product. Decarboxylase activity is observed with substrate as compared to cells containing empty vector.

Alternatively, the decarboxylase candidate may take alpha substituted fumaric acid as a substrate, such as hydroxymethyl fumaric acid. For example this candidate could be tad1 (UMAG_05076) from Ustilago maydis (Geiser et al., 2016).

TABLE M
List of Exemplary decarboxylase sequences

| Gene | Organism | GenBank/Accession number |
|---|---|---|
| CadA (cis-aconitate decarboxylase A) | Aspergillus terreus | BAG49047 |
| CadA (cis-aconitate decarboxylase A) | Aspergillus niger | EAU29420 |
| Stipitatonate Decarboxylase | Talaromyces stipitatus | XP_002341280 |
| FDC1 (Ferulic acid decarboxylase 1) | Saccharomyces cerevisiae | AAB64981 |
| MmgE/PrpD family protein | Halarchaeum acidiphilum | WP_021779749 |
| MmgE/PrpD family protein | Cupriavidus sp. HMR-1 | WP_008644277 |
| CadA (cis-aconitate decarboxylase A) | Mus Musculus | BAC29433 |
| 4-oxalocrotonate decarboxylase | Geobacillus stearothermophilus | ACA01540 |
| 4-oxalocrotonate decarboxylase | Pseudomonas putida | AAA25693 |
| 2-hydroxymuconate-6-semialdehyde hydrolase | Pseudomonas putida | AAA26054 |
| Phosphoenolpyruvate carboxykinase (ATP) | Saccharomyces cerevisiae | CAA31488 |
| MmgE/prpD family protein | Aspergillus terreus NIH2624 | XP_001215146 |
| MmgE/prpD family protein | Bacillus subtilis | BAA08333 |
| MmgE/prpD family protein | Lactobacillus sucicola JCM 15457 | GAJ27510 |
| MmgE/prpD family protein | Bordetella pertussis Tohama I | NP_881740 |
| MmgE/prpD family protein | Bordetella pertussis Tohama I | NP_878944 |
| MmgE/prpD family protein | Bacillus firmus DS1 | EWG10287 |
| MmgE/prpD family protein | Rhodococcus opacus PD630 | AHK34564 |
| MmgE/prpD family protein | Rhodococcus rhodochrous | WP_016693543 |

Construction of Recombinant Microorganism for Production of Alpha (Hydroxymethyl) Acrylic Acid Ester Starting from Alpha (Hydroxymethyl) Acrylic Acid.

In addition to the DNA fragments listed above in this example, the DNA fragment encoding an esterase (FIG. 12, row H) is included. Specifically, the esterase is carboxylesterase (EC 3.1.1.1) from E coli. The resulting plasmid that successfully transcribes all pathway genes for production of alpha (hydroxymethyl) acrylic acid ester starting from alpha (hydroxymethyl) acrylic acid is transformed into the organism that produces alpha (hydroxymethyl) acrylic acid, described above.

Additionally, expression of a DNA fragment encoding an alpha (hydroxymethyl) acrylic acid transporter improves production of alpha (hydroxymethyl) acrylic acid. Specifically, the transporter gene is msfA encoding the putative Major Facilitator Superfamily protein from Aspergillus terreus (UNIPROT Q0C8L2).
Geiser, Przybilla, Friedrich, Buckel, Wierckx, Blank, and Bolker. Ustilago maydis produces itaconic acid via the unusual intermediate trans-aconitate. Microbiology Biotechnology, 2016. 9,116-129.

### Example 19—Construction of Recombinant Microorganism for Production of Alpha (Hydroxymethyl) Acrylic Acid Starting from Alpha (Hydroxymethyl) 3HP

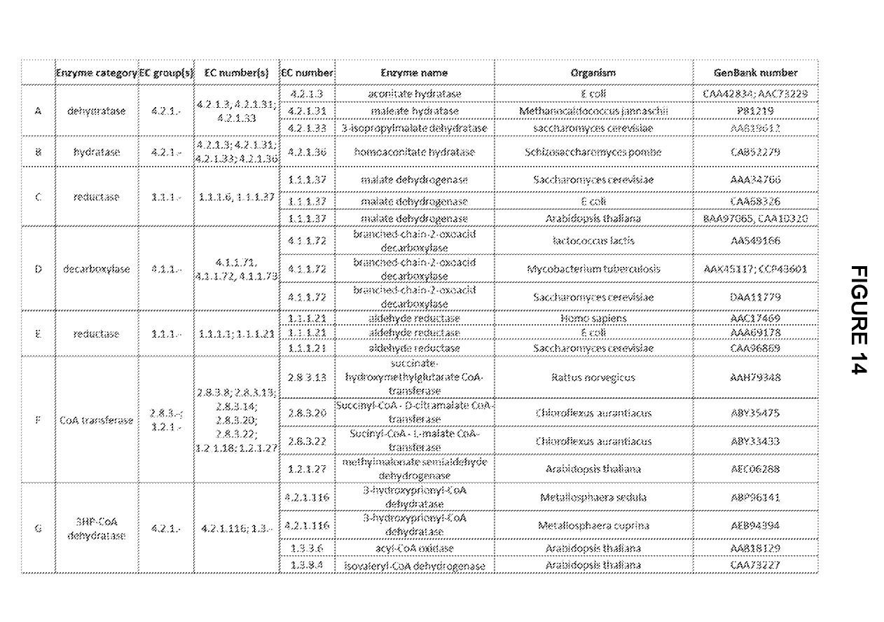

In addition to the DNA fragments listed in Example 9, the DNA fragments encoding coA transferase (FIG. 14, row F), 3HP-CoA dehydratase (FIG. 14, row G), and CoA transferase (FIG. 14, row F) are included. Specifically, the CoA transferase is succinyl-CoA—D-citramalate CoA transferase (EC 2.8.3.20) from Chloroflexus aurantiacus, the 3HP-CoA dehydratase is 3-hydroxyprionyl-CoA dehydratase (EC 4.2.1.116) from Metallosphaera sedula, and the CoA transferase is succinate-hydroxymethylglutarate CoA-transferase (2.8.3.13) from Rattus norvegicus. The resulting plasmid successfully transcribes all pathway genes for production of alpha (hydroxymethyl) acrylic acid starting from alpha (hydroxymethyl) 3-hydroxypropionic acid.

The genes may be selected from M. sedula as described in Teufel et al. These genes are also selected from Arabidopsis thaliana as described in Lucas et al. As described in Lucas et al, Arabidopsis thaliana gene encoding a methylmalonate semialdehyde dehydrogenase would be suitable for CoA transferase activity (FIG. 14, row F) and acyl-CoA dehydrogenase/oxidase and isovaleryl-CoA dehydrogenase will be suitable for 3HP dehydratase activity (FIG. 14, row G).

Additionally, expression of a DNA fragment encoding an alpha (hydroxymethyl) acrylic acid transporter improves production of alpha (hydroxymethyl) acrylic acid. Specifically, the transporter gene is msfA encoding the putative Major Facilitator Superfamily protein from Aspergillus terreus (UNIPROT Q0C8L2).
Lucas, et al. Peroxisomal metabolism of propionic acid and isobutyric acid in plants. JBC 2007. Vol 282, No 34, pp 24980-24989.
Teufel et al. 3-Hydroxypropionyl-coenzyme A dehydratase and acryloyl-coenzyme A reductase, enzymes of the autotrophic 3-hydroxypropionate/4-hydroxybutyrate cycle in the sulfolobales. J Bacteriol 2009 July; 191(14): 4572-4581.

### Example 20—Construction of Recombinant Microorganism for Conversion of Alpha (Hydroxyethyl) C4 Dicarboxylic Acid to Alpha (Hydroxyethyl) Acrylic Acid and Corresponding Lactone

The DNA fragment encoding decarboxylase (FIG. 12, row I) is cloned into an expression vector. Gene candidates and their sequences are shown in FIG. 12 far right column. Specifically, the decarboxylase gene is cis-aconitase decarboxylase, cadA, from Aspergillus niger (EC 4.1.1.6). The resulting plasmid that successfully transcribes all pathway genes is transformed into a recombinant microorganism that produces alpha (hydroxyethyl) malic acid, alpha (hydroxyethyl) maleic acid, or alpha (hydroxyethyl) fumaric acid as described in Example 5.

Additionally, expression of a DNA fragment encoding an alpha (hydroxyethyl) acrylic acid transporter improves production of alpha (hydroxyethyl) acrylic acid. Specifically, the transporter gene is msfA encoding the putative Major Facilitator Superfamily protein from Aspergillus terreus (UNIPROT Q0C8L2).

Construction of Recombinant Microorganism for Production of Tulipalin Starting from Alpha (Hydroxyethyl) Acrylic Acid.

In addition to the DNA fragments listed above in this example, the DNA fragment encoding a lactonase (FIG. 12, row G) is included. Specifically, the esterase is 1,4-lactonase (EC 3.1.1.25) from Homo sapiens. The resulting plasmid that successfully transcribes all pathway genes for production of tulipalin starting from alpha (hydroxyethyl) acrylic acid is transformed into the organism that produces alpha (hydroxyethyl) acrylic acid, described above.

### Example 21—Construction of Recombinant Organism for Production of Alpha-Substituted Acrylic Acid from Alpha-Substituted Acetic Acid

The microorganism expresses all enzymes necessary to convert alpha-substituted acetic acid to alpha-substituted acrylic acid. In one embodiment, 3-hydroxypropionic acid is converted to alpha-hydroxymethyl 3-hydroxypropionic acid. The DNA fragments encoding CoA transferase (FIG. 16, row A), CoA carboxylase (FIG. 16, row B), the oxi-reductase (FIG. 16, row I), the reductase (FIG. 16, row J), the CoA transferase (FIG. 16, row L), 3HP CoA-dehydratase (FIG. 16, row E), and CoA transferase (FIG. 16, row F) are cloned into an expression vector. Gene candidates and their sequences are shown in FIG. 16, far right column.

Specific enzymes and references are shown in the table below (Table N) and described in the accompanying text below. Specifically, the CoA transferase (step A) is 3-hydroxypropionyl-CoA synthetase from Metallosphaera sedula, the CoA carboxylase (step B) is propionyl-CoA carboxylase from Rugeria pomeroyi, the oxi-reductase (step I) and malonyl-CoA reductase/succinyl-CoA reductase (step J) is the bifunctional malonyl-CoA reductase from Chloroflexus aurantiacus, the CoA transferase (step L) is 3-hydroxypropionyl-CoA synthetase from Metallosphaera sedula, the 3HP CoA-dehydratase (step E) is from Metallosphaera sedula, and CoA transferase (step F) is succinyl-CoA—L-malate CoA-transferase from Chloroflexus aurantiacus. The resulting plasmid that successfully transcribes all pathway genes is transformed into a microorganism overproducing 3-hydroxypropionic acid. The microorganism overproducing 3-hydroxypropionic acid is described in numerous patents, including WO 2015017721 A1, WP 0242418 A2, and reviewed in Tokuyama et al., 2014. The microorganism may be bacterial or eukaryotic. Hosts include E coli, Klebsiella pneumonia, Pseudomonas dentrificans, and yeast strains including S. cerevisiae.

TABLE N
Enzymes and references for the alpha substituted malonyl CoA pathway.

| | Enzyme | | | |
|---|---|---|---|---|
| | category | Enzyme name | Organism | Reference |
| A | CoA transferase | 3-hydroxypropionyl- | Metallosphaera sedula, | Alber, Kung, and |
| | | CoA synthetase/ | Sulfolobus tokodaii | Fuchs, 2008 |
| | | propionate CoA- | | |
| | | transferase | | |
| B | CoA | propionyl-CoA | Rugeria pomeroyi | Huang et al, 2010 |
| | carboxylase | carboxylase | | |
| C | CoA transferase | 3-hydroxypropionyl- | Metallosphaera sedula, | Alber, Kung, and |
| | | CoA synthetase/ | Sulfolobus tokodaii | Fuchs, 2008 |
| | | propionate CoA- | | |
| | | transferase | | |
| D | reductase; CoA | 3-hydroxyacyl-CoA | Metallosphaera sedula | Hawkins, Adams, |
| | dehydrogenase | dehydrogenase | | and Kelly, 2014 |
| E | 3HP CoA- | 3HP CoA- | Metallosphaera sedula | Teufel et al., 2009 |
| | dehydratase | dehydratase | | |
| F | CoA transferase | succinyl-CoA - L- | Chloroflexus aurantiacus | Friedmann et al., |
| | | malate CoA- | | 2006 |
| | | transferase | | |
| G | carboxylase | 2-oxoglutarate | Hydrogenobactor | Aoshima and |
| | | carboxylase | thermophilus Tk-6 | Igarashi, 2006 |
| H | CoA transferase | 3-hydroxypropionyl- | Metallosphaera sedula, | Alber, Kung, and |
| | | CoA synthetase/ | Sulfolobus tokodaii | Fuchs, 2008 |
| | | propionate CoA- | | |
| | | transferase | | |
| I | oxi-reductase | malonyl-CoA | Metallosphaera sedula, | Alber et al., 2006 |
| | | reductase/succinyl- | Sulfolobus tokodaii, | |
| | | CoA reductase | Chloroflexus aurantiacus | |
| | | | (both I and J) | |
| J | Reductase | Malonic | Metallosphaera sedula, | Atsumi et al., 2010; |
| | | semialdehyde | Chloroflexus aurantiacus | Alber et al., 2006 |
| | | reductase; succinyl- | (both I and J) | |
| | | CoA reductase | | |
| K | dehydratase | 2-methylcitrate | E coli | Brock et al, 2002 |
| | | dehydratase | | |
| L | CoA transferase | 3-hydroxypropionyl- | Metallosphaera sedula, | Alber, Kung, and |
| | | CoA synthetase/ | Sulfolobus tokodaii | Fuchs, 2008 |
| | | propionate CoA- | | |
| | | transferase | | |

Step A: The 3HP CoA synthetase from Metallosphaera sedula has been characterized by Alber et al. 2008 J. Bacteriol. The enzyme is a part of the 3-hydroxypropionate cycle autotrophic CO₂ fixation pathway of this organism. In Chloroflexus aurantiacus, this step is performed by a domain of a tri-functional protein which appears to have evolved independently to perform the same function (Alber and Fuchs, J B C 2002). The domain containing the 3HP CoA synthetase activity could be isolated and expressed or, alternatively, the two domains without the 3HP CoA synthetase activity could be mutated to inhibit their activity.
Step B: The crystal structure of bacterial propionyl-CoA carboxylase has been resolved (Huang et al 2010 Nature). From the structure, the carboxylase transferase active site is observed to be a large canyon. The structure suggests that it may be able to accommodate a somewhat larger substrate. The solved structure allows us to choose amino acids to target to engineer an enzyme that will be able to accommodate the terminal hydroxy group. For example, site-directed mutagenesis will be used to make this portion of the active site more hydrophilic.
Step C: The specificity of 3HP coA synthetase described in Alber et al 2008 was determined by replacing 3-hydroxypropionate in the standard coupled assay with other potential substrates. The 3HP coA synthetase from M. sedula showed activity with a variety of substrates including propionate, acrylate, acetate, and butyrate. The 3-HP CoA synthetase from S. tokodaii was also characterized and reported to have activity with propionate, acrylate, acetate, butyrate, glycolate, 3-mercaptopropionate, and 3-chloropropionate. The variety of substrates for these enzymes suggests that the enzyme is promiscuous in its specificity. This apparent promiscuity suggests the enzyme may already possess sufficient CoA transferase activity with our substrate of interest. Another candidate CoA transferase is described in Step F.
Step D: The 3-hydroxybutyrl-CoA dehydrogenase, an enzyme involved in the 3HP/4HB cycle in M. sedula, was purified and characterized as reported by Hawkins, Adams, and Kelly, 2014. In a broader perspective of the enzyme family, the hydroxyacyl coenzyme A dehydrogenase has been studied by many groups for decades, as demonstrated by papers published as early as the 1950s, for example Wakil et al., 1954. The dehydrogenase has a wide specificity, able to catalyze substrates containing from 4 to 12 carbons. The reaction catalyzed by this enzyme is reversible. The extensive knowledge of this enzyme in the field makes this enzyme a promising target for engineering to increase specificity.
Step E: 3HP CoA dehydratase from Metallosphaera sedula has been characterized by Teufel et al. 2009 J. Bacteriol. The enzyme is a part of the 3-hydroxypropionate cycle autotrophic CO₂ fixation pathway of this organism. In Chloroflexus aurantiacus, this step is performed by a domain of a tri-functional protein which appears to have evolved independently to perform the same function (Alber and Fuchs, J B C 2002). The natural reaction is the elimination of water from 3-hydroxypropionyl-CoA to form acryloyl-CoA. The enzyme is also able to catalyze the conversion of 3-hydroxybutyryl-CoA to crotonyl-CoA, suggesting the active site is able to accommodate substrates of different sizes.
Step F: Friedmann et al demonstrated that the succinyl-CoA:L-malate coenzyme A transferase from Chloroflexus aurantiacus is specific in its use of succinyl-CoA as the CoA donor but naturally utilizes more than one CoA acceptor, malate or citramalate. The natural dual function of the enzyme suggests that the pocket is flexible enough to accept substrates of different sizes. Also see description for steps A, C, H, and I which also describe the reversible CoA transferase reactions that are possible candidates for performing this reaction.
Step G: The enzyme 2-oxoglutarate carboxylase was identified by Aoshima et al, 2004 and further characterized by Aoshima and Igarashi, 2006 in Hydrogenobacter thermophilus. The reaction catalyzed by this enzyme is important for the reductive TCA cycle used by autotrophic organisms and requires ATP. The most direct way to monitor this reaction is to detect the product via chromatography. A real-time spectrometer assay can also be used as described in Aoshima and Igarashi 2006. 2-oxoglutarate carboxylase is reported to be structurally similar to pyruvate carboxylase and likely evolved from a common protein. The crystal structure of pyruvate carboxylase has been solved and could be used to engineer increased specificity to the substrate of interest and of the structurally similar 2-oxoglutarate carboxylase (Jitrapakdee et al., 2008).
Step H: See description for Steps C and F.
Step I: The malonyl-CoA reductase from Chloroflexus aurantiacus is bifunctional and is able to catalyze the reduction of the CoA-activated carboxylic acid carboxylic acid and reduction of the semialdehyde (Alber et al., 2006). In contrast, the malonic semialdehyde reductase from M. sedula only catalyzes the reduction from CoA carboxylic acid to semialdehyde, for example succinyl-CoA to succinic semialdehyde (Kockelkorn and Fuchs, 2009). In addition to using succinyl-CoA as a substrate, this enzyme also possesses malonyl-CoA reductase activity. Further characterization of the enzyme suggested that the NADH-dependent enzyme was promiscuous in its selectivity and is related to the well-studied aspartate reductase dehydrogenase.
Step J: As stated above in the description accompanying Step I, the malonyl-CoA reductase from Chloroflexus aurantiacus is bifunctional and is able to catalyze the reduction of the CoA-activated carboxylic acid and reduction of the semialdehyde (Alber et al, 2006). In Metallosphaera sedula, this step is carried out by malonic semialdehyde reductase (Kockelkorn and Fuchs, 2009). A catalytic mechanism is proposed by Alber et al. utilizing a conserved cysteine and histidine (Alber et al, 2006). This information and similarity of the enzyme to other aldehyde dehydrogenases offer insights that will be used to engineer this enzyme to accept our intermediate of interest. Alternatively, E coli expresses several aldehyde reductases that can be screened for activity in this reaction. For example, the aldehyde reductase from E coli, YqhD, has a broad substrate and has been demonstrated to be used in biotech applications (Atsumi et al, 2010). Similarly, the adh2 gene from S. cerevisiae could be used to catalyze this reaction.
Step K: The enzyme prpD from E coli is able to catalyze the dehydration of methylcitrate to 2-methylaconitate (Brock et al, 2002). This enzyme is used in the endogenous pathway to oxidate propionate to pyruvate and is exclusively present when E coli is grown on propionate. Brock et al, showed that E coli prpD has some activity with aconitate. Small amounts of activity were also observed with citrate and isocitrate, suggesting that the enzyme has potential to use different substrates.
The crystal structure of prpD from Salmonella enterica has been solved (Gulick et al, 2002). This structure will be useful to modify the specificity of this step to dehydrate 2-(hydroxymethyl)-3-hydroxypropionate, as required on the pathway to hydroxymethyl acrylic acid.
Step L: See description for steps C and F.

Alternative iterations are constructed to produce alpha (hydroxymethyl) acrylic acid from 3-hydroxypropionic acid. One pathway includes enzymes described in steps A, B, I, C, D, E, and F. Another pathway includes enzymes described in steps A, B, I, J, and K. Another pathway includes enzymes described in steps G, H, I, C, D, E, and F. Another pathway includes enzymes described in steps G, H, I, J, L, E, and F. Another pathway includes enzymes described in steps G, H, I, J, and K. Another pathway includes enzymes described in steps A, B, I, J, L, E, and F.

Additionally, expression of a DNA fragment encoding an alpha (hydroxymethyl) acrylic acid transporter improves production of alpha (hydroxymethyl) acrylic acid. Specifically, the transporter gene is msfA encoding the putative Major Facilitator Superfamily protein from Aspergillus terreus (UNIPROT Q0C8L2).

### Carboxylase Assay

The amount of 3HP-CoA that was converted to hydroxymethylmalonyl-CoA was measured using a coupled reaction resulting in pyruvate accumulation. E coli cells were transformed with either empty vector (ptrc) or RpPCC. Cells were lysed using mechanical disruption using a BeadBeater (BopSpec products, Bartlesville, Okla.) using the manufacturer's instructions. The cell lysate was partially clarified by centrifugation (14,000G for 5 minutes). Protein concentrations of the resulting clarified lysates were measured via BioRad total Protein assay using the manufacturer's instructions. Lysates were normalized by protein concentration with 100 mM potassium phosphate buffer, pH 7.6. The pyruvate-coupled carboxylase reaction assays contained 100 mM potassium phosphate buffer (pH 7.6), 5 μl of pyruvate kinase (2.5 units per μl), 5 mM phosphoenolpyruvate 0.3 mg/mL BSA, 5 mM MsCl₂, 50 mM NaHCO₃, 5 mM ATP, and 5 mM 3HP-CoA substrate. The reaction was started with 25 μl of lysate added to the reaction mix to reach a total volume of 100 μl. Pyruvate accumulation was assessed via HPLC. The lysate expressing RpPCC accumulated pyruvate over time indicated carboxylase of 3HP-CoA to result in hydroxymethyl malonyl-CoA (FIG. 33(b)).
Alber et al. Malonyl-coenzyme A reductase in the modified 3-hydroxypropionate cycle for autotrophic carbon fixation in archaeal Metallosphaera and Sulfolobus spp. J Bacteriology, December 2006, p. 8551-8559.
Alber and Fuchs. Propionyl-coenzyme A synthase from Chloroflexus aurantiacus, a key enzyme of the 3-hydropropionate cycle for autotrophic CO₂ fixation. J Biological Chemistry, 2002. Vol. 277, No. 14, pp 12137-12143.
Alber, Kung, Fuchs. 3-hydroxyprionyl-coenzyme A synthetase from Metallosphaera sedula, an enzyme involved in autotrophic CO₂ fixation. J bacteriology. February 2008, p1383-1389.
Aosihma, Ishii, and Igarashi. A novel biotin protein required for reductive carboxylation of 2-oxoglutarate by isocitrate dehydrogenase in Hydrogenobacter thermophilus TK-6. Mol microbial. 2004 February; 51(3): 791-798.
Aoshima and Igarashi. A novel oxalosuccinate-forming enzyme involved in the reductive carboxylation of 2-oxoglutarate in Hydrogenobacter thermophilus TK-6. Molec Microbio. 2006, 62(3), 748-759.
Atsumi, Wu, Eckl, Hawkins, Buelter, and Liao. Engineering the isobutanol biosynthetic pathway in Escherichia coli by comparison of three aldehyde reductase/alcohol dehydrogenase genes. Appl Microbiol Biotechnol. January 2010; 85(3):651-657.
Brock, Maerker, Schutz, and Volker. Oxidation of propionate to pyruvate in Escherichia coli, Involvement of methylcitrate dehydratase and aconitase. Eur J Biochem, FEBS 2002, 269, 6184-6194.
Friedmann, Steindorf, Alber, and Fuchs. Properties of succinyl-coenzyme A:L-malate coenzyme A transferase and its role in the autotrophic 3-hydroxypropionate cycle of Chloroflexus aurantiacus. J Bacteriology, April 2006, Vol 188, No. 7, p 2646-2655.
Grimek, T. L., Escalante-Semerena, J. C. The acnD genes of Shewenella oneidensis and Vibrio cholerae encode a new Fe/S-dependent 2-methylcitrate dehydratase enzyme that requires prpF function in vivo. J. Bacteriol. 186: 454-462 (2004). [PMID: 14702315]
Gulick, Horswill, Thoden, Escalante-Semerena, and Rayment. Pentaerythritol propoxylate: a new crystallization agent and cryoprotectant induces crystal growth of 2-methylcitrate dehydratase. Acta Cryst, 2002. D58, 306-309.
Hawkins, Adams, and Kelly. Conversion of 4-hydroxybutyrate to acetyl coenzyme A and its anapleurosis in the Metallosphaera sedula 3-hydroxypropionate/4-hydroxybutyrate carbon fixation pathway. Appl Environ Microbiol. 2014 April; 80(8): 2536-2545.
Huang, et al. Crystal structure of the a6b6holoenzyme of propionyl-coenzyme A carboxylase. Nature 2010 Aug. 19; 466(7309):1001-1005.
Jitrapakdee, et al. Structure, mechanism, and regulation of pyruvate carboxylase. Biochem J. 2008 Aug. 1; 413(3):369-387.
Kockelkorn and Fuchs. Malonic semialdehyde reductase, succinic semialdehyde reductase, and succinyl-coenzyme A reductase from Metallosphaera sedula: Enzymes of the autotrophic 3-hydroxypropionate/4-hydroxybutyrate cycle in Sulfolobales. J of Bacteriology, October 2009, p. 6352-6362.
Teufel et al. 3-hydroxypropionyl-coenzyme A dehydratase and acryloyl-coenzyme A reductase, enzymes of the autotrophic 3-hydroxypropionate/4-hydroxybutyrate cycle in Sulfolobales. J Bacteriol. 2009 July; 191(14):4572-81.
Tokuyama et al., Increased 3-hydroxypropionic acid production from glycerol, by modification of central metabolism in Escherichia coli. Microbiol Cell Factories 2014, 13:64.
Wakil, Green, and Mahler. Studies on the fatty acid oxidizing system of animal tissues, VI. β-hydroxyacyl coenzyme A dehydrogenase. J Biological Chemistry, August 1953; 207, 631-638.

### Example 22—Method of Converting Alpha-Substituted 3-Hydroxypropionic Acid to Alpha-Substituted Acrylic Acid

In the case of the microorganism producing alpha-substituted 3-hydroxymethyl acid, such as in Examples 9 and 21, this compound is dehydrated to alpha-substituted acrylic acid. In one embodiment, alpha-hydroxymethyl 3-hydroxypropionic acid (HM3HP) is dehydrated to alpha-hydroxymethyl acrylic acid (HMA). A known amount of HM3HP was dissolved into buffered solution. The solution was split into three aliquots which were adjusted to either pH 3, pH 5, or pH 7. Samples were incubated at −20° C., 30° C., or 70° C. overnight. NMR analysis was used to measure the amount of HM3HP that was dehydrated to HMA. The most conversion to HMA was observed at the pH 10 (Table 0). The results indicate that more basic pH drives conversion of HM3HP to HMA. The pH of the solution had more effect on conversion to HMA than did changes in temperature.

TABLE O
Comparison of hydroxymethyl acrylic acid (HMA)
converted from alpha-hydroxymethyl 3-hydroxypropionc
acid at different temperature and pH.

| | |
|---|---|
| | Relative HMA |
| Temperature | levels |

| | | | |
|---|---|---|---|
| pH 3 | −20 | C. | 0.00 |
| | 30 | C. | 0.00 |
| | 70 | C. | 0.00 |
| pH 7 | −20 | C. | 0.13 |
| | 30 | C. | 0.13 |
| | 70 | C. | 0.18 |
| pH 10 | −20 | C. | 0.81 |
| | 30 | C. | 1.18 |
| | 70 | C. | 1.00 |

### Example 23—Construction of Recombinant Microorganism for Production of Alpha (Hydroxyethyl) Acrylic Acid Starting from Itaconic Acid

The microorganism used to for production of alpha (hydroxyethyl) malic acid from itaconic acid can be selected from hosts that produce itaconic acid as described in Example 4, including yeast and filamentous fungi as well as bacteria. Such organisms include S. cerevisiae, E. coli, as well as fungal strains, such as Aspergillus and Ustilago strains. Specific fungal strains include A. niger, A. terreus, and Ustilago maydis. Itaconic acid production is natural to the organism or produced by expressing and/or overexpressing the relevant genes, endogenous and/or exogenous including citrate synthase, aconitase, and cis-aconitate decarboxylase (Bonnarme et al, 1995; Huang et al, 2014; Vuoristo et al, 2014).

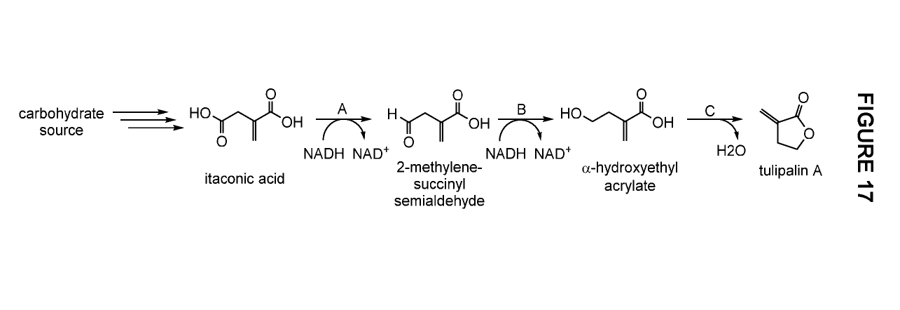

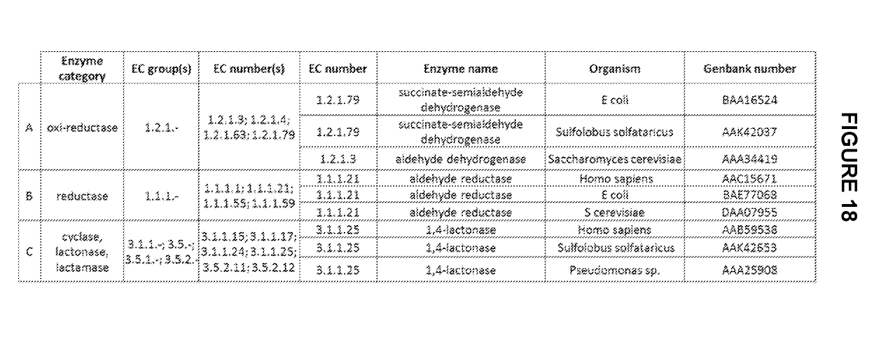

The DNA fragment encoding an oxi-reductase (FIG. 18, row A) and a reductase (FIG. 18, row B) are cloned into an expression vector. Specifically, the oxi-reductase is succinate-semialdehyde dehydrogenase from E coli and the reductase is aldehyde reductase from E. coli or S. cerevisiae. Reductase activity can be assayed as described in Kockelkom and Fuchs, 2009. The plasmid encoding activity for the conversion of itaconic acid to alpha (hydroxyethyl) acrylic acid, as illustrated in FIG. 17, is transformed into the host organism described above. Additionally, expression of a DNA fragment encoding an alpha (hydroxyethyl) acrylic acid transporter improves production of alpha (hydroxyl methyl) acrylic acid. Specifically, the transporter gene is msfA encoding the putative Major Facilitator Superfamily protein from Aspergillus terreus (UNIPROT Q0C8L2).

In addition to the DNA fragments listed above in this example, the DNA fragment encoding a lactonase (FIG. 18, row C) is included. Specifically, the lactonase is 1,4-lactonase (EC 3.1.1.25) from Homo sapiens. The resulting plasmid that successfully transcribes all pathway genes for production of tulipalin starting from itaconic acid is transformed into the itaconic producing organism described above.
Bonnarme, Gillet, Sepulchre, Role, Beloeil and Ducrocq. Itaconate biosynthesis in Aspergillus terreus. J Bacterio, 1995. 177(12): 3573.
Huang, Lu, Li, Li, and Li. Improving itaconic acid production through genetic engineering of an industrial Aspergillus terreus strain. Microbial Cell Factories 2014, 13:1 19.
Kockelkorn and Fuchs. Malonic semialdehyde reductase, succinic semialdehyde reductase, and succinyl-coenzyme A reductase from Metallosphaera sedula: Enzymes of the autotrophic 3-hydroxypropionate/4-hydroxybutyrate cycle in Sulfolobales. J of Bacteriology, October 2009, p. 6352-6362.
Vuoristo, Mars, Sangra, Springer, Eggink, Sanders, and Weusthuis. Metabolic engineering of itaconate production in Escherichia coli. Appl Microbiol Biotechnol, 2014.

### Example 24—Method of Fermenting and Separating Functionalized Alpha Substituted Acrylic Acids

Fermentation methods for production of functionalized alpha substituted acrylic acids are carried out as described in Example 10. Separation of functionalized alpha substituted acrylic acids is performed via methods similar to those used to separate itaconic acid from fermentation broth, such as anion exchange, reverse osmosis, crystallization, and membrane extraction (U.S. Pat. No. 3,544,455A, CN 102940992A, CN 101643404B). More specifically, methods to prepare hydroxyalkyl acrylic acids are described in references JP10218835A and JP10218834A.

1. A recombinant microorganism comprising functionalized alpha substituted C4 dicarboxylic acid, wherein the functional alpha substitution is selected from an alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and at least one recombinant nucleic acid sequence encoding at least one enzyme selected from a transaminase (FIG. 4, row A), a synthase (FIG. 4, row B), a dehydratase (FIG. 4, row C), a hydratase (FIG. 4, row D), a dehydrogenase (FIG. 4, row E), a cis-trans isomerase (FIG. 4, row F), a cyclase, lactonase, lactamase (FIG. 4, row G), an esterase (FIG. 4, row H), a reductase (FIG. 6, row C), a decarboxylase (FIG. 6, row D), and an aldehyde reductase (FIG. 6, row E).

2. The recombinant microorganism according to claim 1, wherein the functionalized alpha substituted C4 dicarboxylic acid is selected from an alpha-(hydroxymethyl) malic acid, an alpha-(2-hydroxypropyl) malic acid, an alpha-(1-hydroxyethyl) malic acid and an alpha-(2-hydroxyethyl) malic acid.

3. The recombinant microorganism according to claim 1, further comprising a functionalized alpha substituted C4 dicarboxylic acid, wherein the functionalized alpha substitution additionally comprises a spacer comprising one or more carbon atoms associated with two hydrogen atoms.

4. The recombinant microorganism according to claim 3, wherein the spacer is selected from —(CH₂)₂—, —(CH₂)₃— or —(CH₂)₄—.

5. The recombinant microorganism according to claim 1, wherein the functionalized alpha substituted C4 dicarboxylic acid comprises a functional group selected from an amino acid side chain.

6. The recombinant microorganism according to claim 1, wherein the functionalized alpha substituted C4 dicarboxylic acid is selected from a functionalized alpha substituted malic, a functionalized alpha substituted maleic, and a functionalized alpha substituted fumaric acid.

7. The recombinant microorganism according to claim 1, wherein the recombinant microorganism comprises at least two functionalized alpha substituted C4 dicarboxylic acids selected from a functionalized alpha substituted malic, a functionalized alpha substituted maleic, and a functionalized alpha substituted fumaric acids.

8. The recombinant microorganism according to claim 1, wherein the recombinant microorganism comprises at least three functionalized alpha substituted C4 dicarboxylic acids selected from a functionalized alpha substituted malic, a functionalized alpha substituted maleic, and a functionalized alpha substituted fumaric acids.

9. The recombinant microorganism according to claim 5, wherein the at least two functionalized alpha substituted C4 dicarboxylic acids comprise the same functional substitution group.

10. The recombinant microorganism according to any one of claims 1 to 9, further comprising a beta functionalized alpha keto acid.

11. The recombinant microorganism according to any one of claims 1 to 10, further comprising a functionalized alpha substituted 3-hydroxypropionic acid.

12. The recombinant microorganism according to any one of claims 1 to 11, wherein the recombinant microorganism selectively overproduces at least one amino acid.

13. The recombinant microorganism according to claim 12, wherein the recombinant microorganism produces at least 0.1 g/L/hour of an amino acid selected from histidine, arginine, asparagine, lysine, methionine, cysteine, phenylalanine, threonine, glutamate, glutamine, tryptophan, selenocysteine, serine, homoserine, homothreonine, tyrosine, valine, leucine and isoleucine.

14. The recombinant microorganism according to any one of claims 1 to 11, wherein the recombinant microorganism selectively overproduces the functionalized alpha substituted C4 dicarboxylic acid.

15. The recombinant microorganism according to any one of claims 1 to 14, wherein the recombinant microorganism produces at least 0.1 g/L/hour of the functionalized alpha substituted C4 dicarboxylic acid.

16. The recombinant microorganism according to claim 10, wherein the recombinant microorganism produces at least 0.1 g/L/hour of the functionalized beta substituted alpha keto acid.

17. The recombinant microorganism according to claim 8, wherein the recombinant microorganism produces at least 0.1 g/L/hour of the functionalized alpha substituted fumaric acid.

18. The recombinant microorganism according to claim 8, wherein the recombinant microorganism produces at least 0.1 g/L/hour of a functionalized alpha substituted maleic acid.

19. The recombinant microorganism according to claim 8, wherein the recombinant microorganism produces at least 0.1 g/L/hour of the functionalized alpha substituted malic acid.

20. The recombinant microorganism according to any one of claims 1 to 19, further comprising a recombinant nucleic acid sequence encoding an organic acid transporter.

21. The recombinant microorganism according to any one of claims 1 to 20, wherein the recombinant microorganism is a prokaryote.

22. The recombinant microorganism according to any one of claims 1 to 20, wherein the recombinant microorganism is a eukaryote.

23. A process of making a functionalized alpha substituted malic acid comprising culturing the recombinant microorganism of any one of claims 1 to 22 in the presence of a carbohydrate; and separating the functionalized alpha substituted malic acid or salt thereof.

24. A process of making a functionalized substituted maleic acid comprising culturing the recombinant microorganism of any one of claims 1 to 22 in the presence of a carbohydrate; and separating the functionalized substituted maleic acid or salt thereof.

25. A process of making a functionalized beta substituted keto acid comprising culturing the recombinant microorganism of any one of claims 1 to 22 in the presence of a carbohydrate; and separating the functionalized beta substituted keto acid or salt thereof.

26. A process of making an alpha substituted 3-hydroxypropionic acid comprising culturing the recombinant microorganism of any one of claims 1 to 22 in the presence of a carbohydrate; and separating the functionalized alpha substituted 3-hydroxypropionic acid or salt thereof.

27. A method for making a functionalized alpha substituted acrylate, or a salt or ester thereof, the method comprising contacting a functionalized alpha substituted C4 dicarboxylic acid or functionalized alpha substituted 3-hydroxypropionic acid, or a salt, ester, or lactone thereof, with a metal catalyst.

28. A method for making a compound of Formula I: 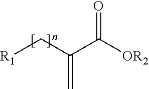
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and R₂ is individually selected from H and a protecting group, and n is equal to 1 or greater;
the method comprising contacting a metal catalyst with a composition comprising a compound of Formula II, III, or IV: 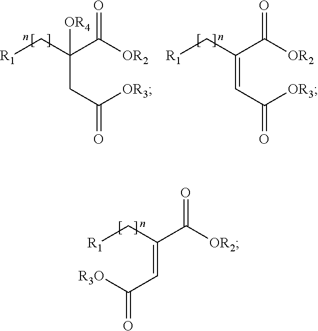
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and protecting groups thereof, and R₂, R₃, R₄ is individually selected from H and a protecting group and n is equal to 1 or greater.

29. A method for making a compound of Formula V: 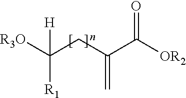
or a salt thereof,
wherein:
each R¹ is selected from H or CH₃, and R² and R³ are selected from H or a protecting group, and n is equal to 1 or greater;
the method comprising contacting a metal catalyst with composition comprising a compound of Formula VI, VII, or VIII: 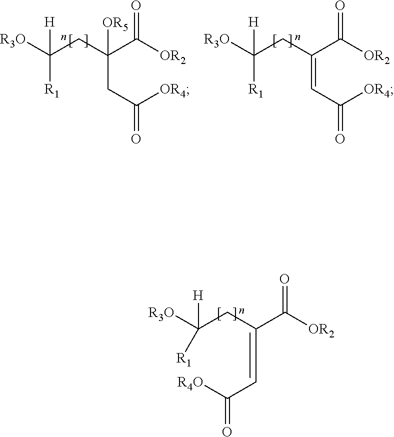
or a salt thereof,
wherein:
each R₁ is selected from H or CH₃, and R², R³, and R⁴ are individually selected from H or a protecting group and n is equal to 1 or greater.

30. A method for making a derivative of a functionalized alpha substituted C4 dicarboxylic acid and a compound having formula I, the method comprising contacting a compound selected from formula II, III and IV or a salt, ester or lactone thereof, with a metal catalyst, wherein said derivative of functionalized alpha substituted C4 dicarboxylic acid is selected from: 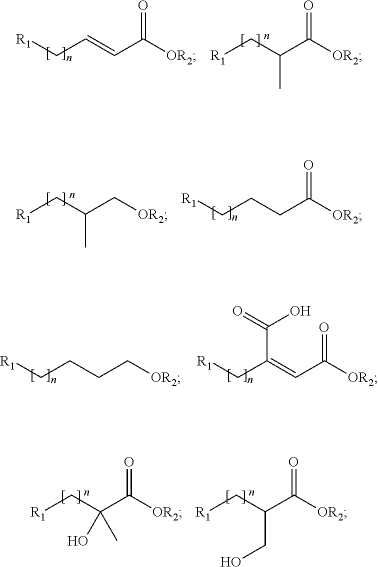
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and R₂ is individually selected from H and a protecting group, and R² is individually selected from H and a protecting group, and n is equal to 1 or greater.

31. A method for making a composition comprising a compound having a Formula I: 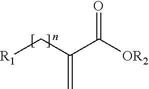
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and protecting groups thereof, R₂ is individually selected from H and a protecting group, and n is equal to 1 or greater, when n is greater than 1, R₁ can be selected from a carboxylate or methyl;
and at least one derivative of a functionalized alpha substituted C4 carboxylic acid;
the method comprising contacting a metal catalyst with a composition comprising a compound of Formula II, III, or IV: 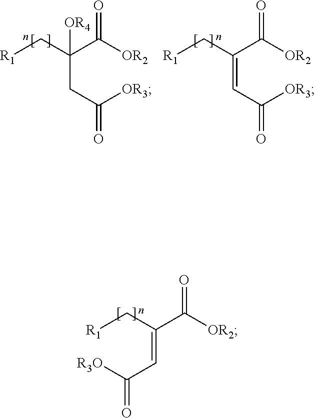
or a salt thereat,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R², R³, R⁴ is individually selected from H and a protecting group and n is equal to 1 or greater.

32. The method according to claim 31, wherein the at least one compound comprises at least two derivatives of functionalized alpha substituted C4 carboxylic acids.

33. A method for making a compound having a Formula I: 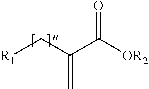
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate and protecting groups thereof, R₂ is individually selected from H and a protecting group, and n is equal to 1 or greater;
the method comprising: contacting a metal catalyst with a functionalized alpha substituted dicarboxylic acid selected from: Formula II, III, or IV, or salts or esters thereof, wherein the beta carboxylate of the functionalized alpha substituted dicarboxylic acid is selectively decarboxylated.

34. A method for making a hydroxyalkyl alpha substituted acrylic acid, or a salt, lactone or ester thereof, the method comprising contacting a hydroxyalkyl alpha substituted C4 dicarboxylic acid, or a salt, ester, or lactone thereof, with a metal catalyst.

35. A method for making a compound of Formula I: 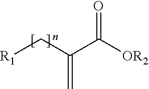
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1 and protecting groups thereof, and R₂ is individually selected from H and a protecting group, and n is equal to 1 or greater;
the method comprising contacting a metal catalyst with a composition comprising a functionalized alpha substituted malic acid, a functionalized alpha substituted maleic acid, a functionalized alpha substituted fumaric acid or a salt, ester or lactone thereof.

36. The method of any one of claims 27-35, wherein the metal catalyst is a heterogeneous catalyst.

37. The method of any one of claims 27-35, wherein the metal catalyst comprises a metal selected from the group consisting of Ni, Pd, Pt, Cu, Zn, Rh, Ru, Bi, Fe, Co, Os, Ir, V, and mixtures of two or more thereof.

38. The method of any one of claims 27-35, wherein the metal catalyst comprises a metal selected from the group consisting of Cu or Pt.

39. The method of claim 38, wherein the metal catalyst comprises Cu.

40. The method of claim 38, wherein the metal catalyst comprises Pt.

41. The method of any one of claims 27-35, wherein the metal catalyst is a supported catalyst.

42. The method of any one of claims 27-35, wherein the metal catalyst comprises a promoter.

43. The method of method 42, wherein the promoter comprises sulfur.

44. The method of any one of claims 27-43, wherein the method is performed at a temperature of at least about 100° C.

45. The method of any one of claims 27-43, wherein the method is performed at a temperature of about 100° C. to about 250° C.

46. The method of any one of claims 27-43, wherein the method is performed at a temperature of about 150° C. to about 200° C.

47. The method of any one of claims 27-46, wherein the metal catalyst is activated prior to the contacting.

48. The method of claim 47, wherein the metal catalyst is activated under hydrogen gas.

49. The method of any one of claims 27-47, wherein the metal catalyst is substantially free of hydrogen.

50. The method of any one of claim 47 or 48, wherein the metal catalyst is activated at a temperature of about 100° C. to about 200° C.

51. A composition comprising a functionalized alpha substituted acrylic acid or a salt, ester or lactone thereof, and one or more compounds selected from an alpha-(hydroxymethyl) malic acid, an alpha-(2-hydroxypropyl) malic acid, an alpha-(1-hydroxyethyl) malic acid and an alpha-(2-hydroxyethyl) malic acid or salts or esters thereof.

52. The composition according to claim 51, further comprising one or more derivatives of functionalized alpha substituted C4 carboxylic acids.

53. A method for making a compound of Formula I: 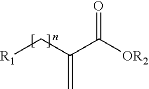
or a salt thereof,
wherein: each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and R₂ is individually selected from H and a protecting group, and n is equal to 1 or greater;
the method comprising heating a functionalized alpha substituted 3HP to dehydrate the functionalized alpha substituted 3HP and produce a compound having the Formula I.

54. A recombinant microorganism comprising a functionalized alpha substituted acrylic acid, wherein the functional alpha substitution is selected from an alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>2, and at least one recombinant nucleic acid sequence encoding at least one enzyme selected from a transaminase (FIG. 12, row A), a synthase (FIG. 12, row B), a dehydratase (FIG. 12, row C), a hydratase (FIG. 12, row D), a dehydrogenase (FIG. 12, row E), a cis-trans isomerase (FIG. 12, row F), a cyclase, lactonase, lactamase (FIG. 12, row G), an esterase (FIG. 12, row H), a decarboxylase (FIG. 12, row I), a reductase (FIG. 14, row C), a decarboxylase (FIG. 14, row D), a reductase (FIG. 14, row E), a CoA transferase (FIG. 14, row F), and a 3HP-CoA dehydratase (FIG. 14, row G).

55. The recombinant microorganism according to claim 54, further comprising a functionalized alpha substituted C4 dicarboxylic acid and wherein the functionalized alpha substituted C4 dicarboxylic acid is selected from an alpha-(hydroxymethyl) malic acid, an alpha-(2-hydroxypropyl) malic acid, an alpha-(1-hydroxyethyl) malic acid and an alpha-(2-hydroxyethyl) malic acid.

56. The recombinant microorganism according to claim 55, wherein the functionalized alpha substitution additionally comprises a spacer comprising one or more carbon atoms associated with two hydrogen atoms.

57. The recombinant microorganism according to claim 56, wherein the spacer is selected from —(CH₂)₂—, —(CH₂)₃— or —(CH₂)₄—.

58. The recombinant microorganism according to any one of claims 54 to 57, wherein the functionalized alpha substituted acrylic acid comprises a functional group selected from an amino acid side chain.

59. The recombinant microorganism according to claim 54, further comprising a functionalized alpha substituted C4 dicarboxylic acid and wherein the functionalized alpha substituted C4 dicarboxylic acid is selected from a functionalized alpha substituted malic, a functionalized alpha substituted maleic, and a functionalized alpha substituted fumaric acid.

60. The recombinant microorganism according to claim 59, wherein the recombinant microorganism comprises at least two functionalized alpha substituted C4 dicarboxylic acids selected from a functionalized alpha substituted malic, a functionalized alpha substituted maleic, and a functionalized alpha substituted fumaric acids.

61. The recombinant microorganism according to claim 59, wherein the recombinant microorganism comprises at least three functionalized alpha substituted C4 dicarboxylic acids selected from a functionalized alpha substituted malic, a functionalized alpha substituted maleic, and a functionalized alpha substituted fumaric acids.

62. The recombinant microorganism according to claim 60, wherein the at least two functionalized alpha substituted C4 dicarboxylic acids comprise the same functional substitution group.

63. The recombinant microorganism according to any one of claims 54 to 62, further comprising a beta functionalized alpha keto acid.

64. The recombinant microorganism according to any one of claims 54 to 63, further comprising a functionalized alpha substituted 3-hydroxypropionic acid.

65. The recombinant microorganism according to any one of claims 54 to 64, wherein the recombinant microorganism selectively overproduces at least one amino acid.

66. The recombinant microorganism according to claim 65, wherein the recombinant microorganism produces at least 0.1 g/L/hour of an amino acid selected from histidine, arginine, asparagine, lysine, methionine, cysteine, phenylalanine, threonine, glutamate, glutamine, tryptophan, selenocysteine, serine, homoserine, homothreonine, tyrosine, valine, leucine and isoleucine.

67. The recombinant microorganism according to claim 59, wherein the recombinant microorganism selectively overproduces the functionalized alpha substituted C4 dicarboxylic acid.

68. The recombinant microorganism according to claim 59, wherein the recombinant microorganism produces at least 0.1 g/L/hour of the functionalized alpha substituted C4 dicarboxylic acid.

69. The recombinant microorganism according to claim 59, wherein the recombinant microorganism produces at least 0.1 g/L/hour of the functionalized beta substituted alpha keto acid.

70. The recombinant microorganism according to claim 59, wherein the recombinant microorganism produces at least 0.1 g/L/hour of the functionalized alpha substituted fumaric acid.

71. The recombinant microorganism according to claim 59, wherein the recombinant microorganism produces at least 0.1 g/L/hour of a functionalized alpha substituted maleic acid.

72. The recombinant microorganism according to claim 59, wherein the recombinant microorganism produces at least 0.1 g/L/hour of the functionalized alpha substituted malic acid.

73. The recombinant microorganism according to any one of claims 54 to 72, further comprising a recombinant nucleic acid sequence encoding an organic acid transporter.

74. The recombinant microorganism according to any one of claims 54 to 73, wherein the recombinant microorganism is a prokaryote.

75. The recombinant microorganism according to any one of claims 54 to 73, wherein the recombinant microorganism is a eukaryote.

76. A process of making a functionalized alpha substituted acrylic acid comprising culturing the recombinant microorganism of any one of claims 54 to 75 in the presence of a carbohydrate; and separating the functionalized alpha substituted acrylic acid or salt thereof.

77. A recombinant microorganism comprising a hydroxymethyl malonyl-CoA and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a 3HP CoA-dehydratase (FIG. 16, row E), a CoA transferase (FIG. 16, row F), a carboxylase (FIG. 16, row G), a CoA transferase (FIG. 16, row H), an oxi-reductase (FIG. 16, row I), a reductase (FIG. 16, row J), a dehydratase (FIG. 16, row K), and a CoA transferase (FIG. 16, row L).

78. A recombinant microorganism comprising a hydroxymethyl malonyl-CoA and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a 3HP CoA-dehydratase (FIG. 16, row E), a CoA transferase (FIG. 16, row F), a carboxylase (FIG. 16, row G), a CoA transferase (FIG. 16, row H), an oxi-reductase (FIG. 16, row I), a reductase (FIG. 16, row J), and a CoA transferase (FIG. 16, row L).

79. The recombinant microorganism according to claim 77 or 78, further comprising 3-hydroxypropionic acid.

80. The recombinant microorganism according to any one of claims 77 to 79, further comprising a recombinant nucleic acid sequence encoding a transferase.

81. The recombinant microorganism according to any one of claims 77 to 80, wherein the microorganism additionally produces 3-hydroxypropionyl-CoA or hydroxymethyl malonic acid at a rate of greater than 0.1 g/L/hr.

82. The recombinant microorganism according to any one of claims 77 to 80, wherein the microorganism additional produces 3 hydroxypropionic acid at a rate of greater than 0.1 g/L/hr.

83. The recombinant cell according to claim 77, further comprising a hydroxymethyl acrylate.

84. The recombinant microorganism according to any one of claims 77 to 83, wherein the microorganism is a prokaryote.

85. The recombinant microorganism according to any one of claims 77 to 83, wherein the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus.

86. The recombinant microorganism according to any one of claims 77 to 83, wherein the microorganism is a eukaryote.

87. The recombinant microorganism according to any one of claims 77 to 83, wherein the microorganism is selected from Candida, Pichia, Saccharomyces, Schizosaccharomyces, Zygosaccharomyces, Kluyveromyces, Debaryomyces, Pichia, Issatchenkia, Yarrowia and Hansenula. Examples of specific host yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis).

88. A process of making an alpha hydroxymethyl acrylate comprising culturing the recombinant microorganism according to claim 77 in the presence of a carbohydrate; and separating the alpha hydroxymethyl acrylate.

89. A recombinant microorganism comprising a compound selected from 2-methylene-succinyl semialdehyde, alpha-hydroxyethyl acrylate and tulipalin A, and a recombinant nucleic acid sequence encoding an enzyme selected from an oxi-reductase (FIG. 18, Table 7, row A), a reductase (FIG. 18, Table 7, row B), a cyclase (FIG. 18, Table 7, row C), a lactonase (FIG. 18, Table 7, row C), a lactamase (FIG. 18, Table 7, row C) and combinations thereof.

90. The recombinant microorganism according to claim 89, wherein the recombinant microorganism remains viable in the presence of at least 10 g/L of a compound selected from 2-methylene-succinyl semialdehyde, alpha-hydroxyethyl acrylate and tulipalin A.

91. The recombinant microorganism according to claim 89 or 90, wherein the recombinant microorganism produces at least 0.1 g/L/hr of itaconic acid.

92. The recombinant microorganism according to any one of claims 89 to 91, wherein the recombinant microorganism produces at least 0.1 g/L/hr of 2-methylene-succinyl semialdehyde.

93. The recombinant microorganism according to any one of claims 89 to 92, wherein the recombinant microorganism produces at least 0.1 g/L/hr of alpha-hydroxyethyl acrylate.

94. The recombinant microorganism according to any one of claims 89 to 93, wherein the recombinant microorganism produces at least 0.1 g/L/hr of tulipalin A.

95. A method of making a compound selected from 2-methylene-succinyl semialdehyde, alpha-hydroxyethyl acrylate and tulipalin A comprising culturing the recombinant microorganism according to any one of claims 89 to 94 with a carbon source and separating the compound from the fermentation broth.

96. A recombinant microorganism comprising an alpha-substituted 3-hydroxypropionic acid and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a carboxylase (FIG. 16, row G), a CoA transferase (FIG. 16, row H), an oxi-reductase (FIG. 16, row I), and a reductase (FIG. 16, row J).

97. A recombinant microorganism comprising an alpha-substituted malonyl-CoA and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a carboxylase (FIG. 16, row G), a CoA transferase (FIG. 16, row H), an oxi-reductase (FIG. 16, row I), and a reductase (FIG. 16, row J).

98. A recombinant microorganism comprising an alpha-substituted malonic semialdehyde and a recombinant nucleic acid sequence encoding an enzyme selected from a CoA transferase (FIG. 16, row A), a CoA carboxylase (FIG. 16, row B), CoA transferase (FIG. 16, row C), a reductase (FIG. 16, row D), a dehydrogenase (FIG. 16, row D), a carboxylase (FIG. 16, row G), a CoA transferase (FIG. 16, row H), an oxi-reductase (FIG. 16, row I), and a reductase (FIG. 16, row J).

99. The recombinant microorganism according to any one of claims 96 to 98, further comprising an alpha-substituted acetic acid.

100. The recombinant microorganism according to any one of claims 96 to 99, further comprising a recombinant nucleic acid sequence encoding a transferase.

101. The recombinant microorganism according to any one of claims 96 to 100, wherein the microorganism additionally produces an alpha-substituted acetyl-CoA or alpha-substituted malonic acid at a rate of greater than 0.1 g/L/hr.

102. The recombinant microorganism according to any one of claims 96 to 101, wherein the microorganism additional produces an alpha-substituted acetic acid at a rate of greater than 0.1 g/L/hr.

103. The recombinant microorganism according to any one of claims 96 to 102, wherein the microorganism is a prokaryote.

104. The recombinant microorganism according to any one of claims 96 to 102, wherein the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus.

105. The recombinant microorganism according to any one of claims 96 to 102, wherein the microorganism is a eukaryote.

106. The recombinant microorganism according to any one of claims 96 to 102, wherein the microorganism is selected from Candida, Pichia, Saccharomyces, Schizosaccharomyces, Zygosaccharomyces, Kluyveromyces, Debaryomyces, Pichia, Issatchenkia, Yarrowia and Hansenula. Examples of specific host yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis).

107. A method of making an alpha-substituted 3-hydroxypropionic acid of the formula: 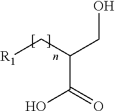
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater;
the method comprising culturing the recombinant microorganism as defined in any one of claims 96 to 106 in the presence of a carbohydrate; and separating the alpha-substituted 3-hydroxypropionic acid or ester or salt thereof.

108. A method for making an alpha-substituted acrylic acid of the formula: 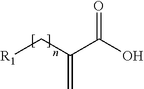
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater;
the method comprising dehydrating an alpha-substituted 3-hydroxypropionic acid to produce the alpha-substituted acrylic acid.

109. The method of claim 108, further comprising producing the alpha-substituted 3-hydroxypropionic acid by the method according to claim 107.

110. A compound of the formula: 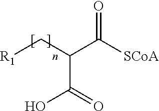
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater.

111. A compound of the formula: 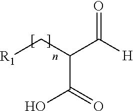
or a salt thereof,
wherein:
each R₁ is selected from alkyl (longer than methyl), hydroxy, hydroxyalkyl, branched hydroxyalkyl, alkoxy, branched alkyl, amino, branched amino alkyl, phenyl, alkyl substituted phenyl, —S—, —SH, —SeH, —Se—, hydroxyaromatic, aminoaromatic, formyl, carbamoyl, indolyl, guanidinyl, and carboxylate when n>1, and n is equal to 1 or greater.

112. The compound of claim 110 or 111, wherein R₁ is hydroxy.

113. The compound of any one of claims 110 to 112, wherein n is 1 or 2.

114. The compound or claim 110 or 111, wherein R₁ is hydroxy and n is 1.

115. The compound or claim 110 or 111, wherein R₁ is hydroxy and n is 2.
